(19) United States
(12) Patent Application Publication
(10) Pub. No.: US 2005/0084872 A1
(43) Pub. Date: Apr. 21, 2005

(54) Methods for determining whether an agent possesses a defined biological activity

(76) Inventors: Pek Yee Lum, Seattle, WA (US); Yejun Tan, Seattle, WA (US); Hongyue Dai, Bothell, WA (US); Eric Stanley Muise, Jersey City, NJ (US); Joel P. Berger, Hoboken, NJ (US); John R. Thompson, Scotch Plains, NJ (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(21) Appl. No.: 10/764,420

(22) Filed: Jan. 23, 2004

Related U.S. Application Data
(60) Provisional application No. 60/442,797, filed on Jan. 24, 2003.
(60) Provisional application No. 60/474,413, filed on May 30, 2003.

(51) Int. Cl. C12Q 1/68; G06F 19/00; G01N 33/48; G01N 33/50

(52) U.S. Cl. 435/6; 702/20

(57) ABSTRACT

In one aspect, the present invention provides methods for determining whether an agent (e.g., candidate drug) possesses a biological activity. In another aspect, the present invention provides populations of nucleic acid molecules useful in the practice of the present invention as probes for measuring the level of expression of populations of genes.

### CROSS-REFERENCES TO RELATED APPLICATIONS

This application claims the benefit of Provisional Application No. 60/442,797, filed Jan. 24, 2003, and Provisional Application No. 60/474,413, filed May 30, 2003.

### FIELD OF THE INVENTION

The present invention relates to methods for screening biologically active agents, such as candidate drug molecules, to identify agents that possess a defined biological activity.

### BACKGROUND OF THE INVENTION

Identifying new drug molecules for treating human diseases is a time consuming and expensive process. A candidate drug molecule is usually first identified in a laboratory using an assay for a desired biological activity. The candidate drug is then tested in animals to identify any adverse side effects that might be caused by the drug. This phase of preclinical research and testing may take more than five years. See, e.g., J. A. Zivin, Understanding Clinical Trials, Scientific American, ps. 69-75 (April 2000). The candidate drug is then subjected to extensive clinical testing in humans to determine whether it continues to exhibit the desired biological activity, and whether it induces undesirable, perhaps fatal, side effects. This process may take up to a decade. Id.

Adverse effects are often not identified until late in the clinical testing phase when considerable expense has been incurred testing the candidate drug. There is a need, therefore, for methods that increase the likelihood of identifying candidate drugs that possess a desirable biological activity, and which do not cause adverse side effects, early in the testing process, thereby reducing the amount of time and resources expended during drug testing.

### SUMMARY OF THE INVENTION

In accordance with the foregoing, in one aspect the present invention provides methods for determining whether an agent possesses a defined biological activity. Each method of this aspect of the invention includes the steps of: (a) making at least one comparison from the group consisting of: (1) comparing an efficacy value of the agent to at least one reference efficacy value to yield an efficacy comparison result, wherein each efficacy value represents at least one expression pattern of the same efficacy-related population of genes, or at least one expression pattern of the same efficacy-related population of proteins; (2) comparing a toxicity value of the agent to at least one reference toxicity value to yield a toxicity comparison result, wherein each toxicity value represents at least one expression pattern of the same toxicity-related population of genes, or at least one expression pattern of the same toxicity-related population of proteins; (3) comparing a classifier value of the agent to at least one reference classifier value to yield a classifier comparison result, wherein each classifier value represents at least one expression pattern of the same classifier population of genes, or at least one expression pattern of the same classifier population of proteins; and (b) using the comparison result(s) obtained in step (a) to determine whether the agent possesses the defined biological activity.

The methods of this aspect of the invention can utilize one, two, or all three of the foregoing comparisons identified by numbers (1), (2) and (3). In embodiments of the invention that utilize two or three of the foregoing comparisons, the comparisons can be made in any temporal sequence (e.g., in embodiments of the invention that utilize all three of the foregoing comparisons, comparison (1) can be made before or after comparison (2), and before or after comparison (3)). Optionally, the methods of this aspect of the invention can include the step of first identifying one or more of the efficacy-related population of genes or proteins, toxicity-related population of genes or proteins, and/or classifier population of genes or proteins. The foregoing populations of genes or proteins can be identified, for example, by using the methods disclosed herein for identifying an efficacy-related population of genes or proteins, a toxicity-related population of genes or proteins, and/or a classifier population of genes or proteins.

In some embodiments of the methods of this aspect of the invention, the defined biological activity is the ability to affect a biological process in vivo, and at least one of the efficacy value of the agent, the toxicity value of the agent and the classifier value of the agent is/are calculated from gene expression levels, and/or protein expression levels, measured in living cells cultured in vitro. In some embodiments of the methods of this aspect of the invention, the defined biological activity is the ability to affect a biological process in a first living tissue, and at least one of the efficacy value of the agent, the toxicity value of the agent and the classifier value of the agent is/are calculated from gene expression levels, and/or protein expression levels, measured in a second living tissue, wherein the first living tissue is a different type of tissue than the second living tissue.

The methods of this aspect of the invention are useful in any situation in which it is desirable to know whether an agent possesses a defined biological activity in a living thing (e.g., prokaryotic cell, eukaryotic cell, plant or animal). For example, the methods of this aspect of the invention are useful in the preclinical stage of drug discovery to identify chemical agents that possess a desired biological activity (e.g., a biological activity that ameliorates the symptoms of a disease), but which elicit few, if any, undesirable side effects when administered to a living organism, such as to a human being or other mammal.

In another aspect, the present invention provides populations of nucleic acid molecules that are useful in the practice of the methods of the present invention as probes for measuring the level of expression of members of a classifier population of genes, or an efficacy-related population of genes, or a toxicity-related population of genes, wherein the classifier population of genes, the efficacy-related population of genes, and the toxicity-related population of genes are each useful for identifying agonists, or partial agonists, of PPARγ. In a related aspect, the present invention provides classifier populations of genes, efficacy-related populations of genes, and toxicity-related populations of genes that are useful in the practice of the methods of the invention for identifying agonists, or partial agonists, of PPARγ.

In yet another aspect, the present invention provides methods for identifying an efficacy-related population of genes or proteins, methods for identifying a toxicity-related population of genes or proteins, and methods for identifying a classifier population of genes or proteins, as described more fully herein. The methods of this aspect of the invention are useful, for example, for identifying efficacy-related populations of genes or proteins, toxicity-related populations of genes or proteins, and classifier populations of genes or proteins, that are useful in the practice of the methods of the invention for determining whether an agent possesses a defined biological activity.

### DETAILED DESCRIPTION OF THE PREFERRED EMBODIMENT

Unless specifically defined herein, all terms used herein have the same meaning as they would to one skilled in the art of the present invention. Practitioners are particularly directed to Sambrook et al. (1989) Molecular Cloning: A Laboratory Manual, 2^{nd} ed., Cold Spring Harbor Press, Plainsview, N.Y.(1989), and Ausubel et al., Current Protocols in Molecular Biology (Supplement 47), John Wiley & Sons, New York (1999), for definitions and terms of the art.

In one aspect, the present invention provides methods for determining whether an agent possesses a defined biological activity. The methods of this aspect of the invention each include the steps of: (1) comparing an efficacy value of the agent to at least one reference efficacy value to yield an efficacy comparison result, wherein each efficacy value represents at least one expression pattern of the same efficacy-related population of genes, or at least one expression pattern of the same efficacy-related population of proteins; (2) comparing a toxicity value of the agent to at least one reference toxicity value to yield a toxicity comparison result, wherein each toxicity value represents at least one expression pattern of the same toxicity-related population of genes, or at least one expression pattern of the same toxicity-related population of proteins; (3) comparing a classifier value of the agent to at least one reference classifier value to yield a classifier comparison result, wherein each classifier value represents at least one expression pattern of the same classifier population of genes, or at least one expression pattern of the same classifier population of proteins; and (b) using the comparison result(s) obtained in step (a) to determine whether the agent possesses the defined biological activity.

In the practice of this aspect of the invention, the amounts of nucleic acid gene products (e.g., the amount of mRNA transcribed from a gene, as represented by the amount of cDNA made from the transcribed mRNA) from defined gene populations are measured, or the amounts of proteins in defined protein populations are measured, to yield gene or protein expression patterns that provide information about the effect of an agent on a living thing. It is sometimes desirable to measure protein levels instead of the levels of gene transcripts because the amount of a protein in a living thing may depend on factors in addition to the level of transcriptional activity of the gene that encodes the protein. For example, the amount of a protein in a living thing may be affected by the activity of a specific protease in a living thing, or on the activity of the protein translational apparatus. These factors may be affected by an agent used to treat a living thing.

As used herein, the term “agent” encompasses any physical, chemical, or energetic agent that induces a biological response in a living organism in vivo and/or in vitro. Thus, for example, the term “agent” encompasses chemical molecules, such as candidate therapeutic molecules that may be useful for treating one or more diseases in a living organism, such as in a mammal (e.g., a human being). The term “agent” also encompasses energetic stimuli, such as ultraviolet light. The term “agent” also encompasses physical stimuli, such as forces applied to living cells (e.g., pressure, stretching or shear forces).

The term “biological activity” refers to the ability of an agent to affect (e.g., stimulate or inhibit) one or more biological processes in a living organism. Examples of biological processes include biochemical pathways; physiological processes that contribute to the internal homeostasis of a living organism; developmental processes that contribute to the normal physical development of a living organism; and acute or chronic diseases.

As used herein, the phrase “efficacy value” refers to a value that numerically represents the level of expression, in response to an agent, of one of the following: (1) all of the genes within an efficacy-related population of genes; or (2) all of the proteins within an efficacy-related population of proteins.

As used herein, the phrase “efficacy-related population of genes” refers to a population of genes, present in a living thing, that yields at least one expression pattern, in response to an agent, that correlates (positively or negatively) with the presence of at least one desired biological response caused by the agent in the living thing.

As used herein, the phrase “efficacy-related population of proteins” refers to a population of proteins, present in a living thing, that yields at least one expression pattern, in response to an agent, that correlates (positively or negatively) with the presence of at least one desired biological response caused by the agent in the living thing.

As used herein, the phrase “toxicity value” refers to a value that numerically represents the level of expression, in response to an agent, of one of the following: (1) all of the genes within a toxicity-related population of genes; or (2) all of the proteins within a toxicity-related population of proteins.

As used herein, the phrase “toxicity-related population of genes” refers to a population of genes, present in a living thing, that yields at least one expression pattern, in response to an agent, that correlates (positively or negatively) with the presence of at least one undesirable biological response caused by the agent in the living thing.

As used herein, the phrase “toxicity-related population of proteins” refers to a population of proteins, present in a living thing, that yields at least one expression pattern, in response to an agent, that correlates (positively or negatively) with the presence of at least one undesirable biological response caused by the agent in the living thing.

As used herein, the phrase “classifier value” refers to a value that numerically represents the level of expression, in response to an agent, of one of the following: (1) all of the genes within a classifier population of genes; or (2) all of the proteins within a classifier population of proteins.

As used herein, the phrase “classifier population of genes” refers to a population of genes, present in a living thing, that yields at least two different gene expression patterns caused by at least two different agents. One of the two expression patterns correlates (positively or negatively) with the presence of a first biological response caused by one of the at least two agents. Another of the at least two expression patterns correlates (positively or negatively) with the presence of a second biological response, that is different from the first biological response, caused by another of the at least two agents. Thus, a classifier population of genes is used to classify an agent into one or more classes based upon the expression pattern of the classifier population of genes that is induced by the agent.

As used herein, the phrase “classifier population of proteins” refers to a population of proteins, present in a living thing, that yields at least two different protein expression patterns caused by at least two different agents. One of the two expression patterns correlates (positively of negatively) with the presence of a first biological response caused by one of the at least two agents. Another of the at least two expression patterns correlates (positively or negatively) with the presence of a second biological response, that is different from the first biological response, caused by another of the at least two agents. Thus, a classifier population of proteins is used to classify an agent into one or more classes based upon the expression pattern of the classifier population of proteins that is induced by the agent.

Representative Biological Activities: The methods of this aspect of the invention are useful in any situation in which it is desirable to know whether an agent possesses a defined biological activity in a living thing. The term “living thing” encompasses all unicellular and multicellular organisms (e.g., plants and animals, including mammals, such as human beings), and also encompasses living tissue, and living organs.

The term “biological activity” can refer to a single biological response, or to a combination of biological responses. Representative examples of biological activities include stimulation or suppression of one or more of the following biological processes that affect the concentration of glucose in mammalian blood: uptake, transport, metabolism and/or storage of glucose by living cells. Further representative examples of biological activities include stimulation or suppression of one or more of the following biological processes that affect the concentration of cholesterol in mammalian blood: stimulation or suppression of cholesterol uptake by living cells, and/or cholesterol metabolism by living cells, and/or cholesterol synthesis by living cells. Again by way of non-limiting example, the methods of the invention can be used to identify agents that affect (e.g., stimulate, or inhibit) one or more of the following biological processes or disease states: Alzheimer's disease; schizophrenia; cancerous tumor size; body mass index; inflammation; and cell division rate.

A biological activity can be defined in terms of any measurable effect, or combination of measurable effects, of an agent on a living thing. For example, a biological activity can be defined with reference to stimulation, and/or inhibition, of one or more biological responses; and/or the absolute and/or relative magnitude of stimulation, and/or inhibition, of one, or more, biological responses; and/or the inability to affect (e.g., the inability to stimulate or inhibit) one, or more, biological responses.

Thus, for example, a defined biological activity can be the ability to stimulate a target biological response (e.g., raise the level of high density lipoprotein in human blood). Again by way of example, a defined biological activity can be the combination of the ability to stimulate a target biological response (e.g., raise the level of high density lipoprotein in human blood) without stimulating one, or more, undesirable biological responses (e.g., without increasing blood plasma volume, or without causing liver damage). By way of further example, in the context of comparing numerous agents within a population of agents, the defined biological activity can be the combination of causing the strongest stimulation of a target biological response, while causing the least stimulation of an undesirable biological response (i.e., in this example the agent, within the population of agents, that most strongly stimulates the target biological response, but causes the least stimulation of an undesirable biological response, possesses the defined biological activity).

The use of efficacy values in the practice of the invention: The methods of the invention can include the step of comparing an efficacy value of an agent to at least one reference efficacy value to yield an efficacy comparison result, wherein each efficacy value represents at least one expression pattern of the same efficacy-related population of genes, or at least one expression pattern of the same efficacy-related population of proteins. In some embodiments, an efficacy value of the agent is compared to a scale of efficacy values to yield an efficacy comparison result, wherein each efficacy value represents at least one expression pattern of the same efficacy-related population of genes, or at least one expression pattern of the same efficacy-related population of proteins.

An efficacy value is a value that numerically represents the level of expression, in response to an agent, of one of the following: (1) all of the genes within an efficacy-related population of genes; or (2) all of the proteins within an efficacy-related population of proteins. The population of efficacy-related genes, or the population of efficacy-related proteins, yields an expression pattern, and, therefore, an efficacy value, that correlates (positively or negatively) with the occurrence of one or more desired biological response(s) caused by an agent in a living thing. A representative example of a desired effect in a living thing is the return of an abnormal expression pattern of a population of genes, and/or proteins, and/or non-protein molecules, in a diseased organism, to a normal expression pattern that is characteristic of a healthy organism. A representative example of a desired effect in a human being suffering from, or predisposed to, atherosclerosis is reduction in the concentration of total cholesterol in the subject's blood plasma.

The expression pattern of an efficacy-related population of genes or proteins induced by an agent, and, therefore, the efficacy value calculated from the induced gene expression pattern, or protein expression pattern, provides an indication of the extent to which an agent induces one or more desired effect(s) in a living thing. Thus, the effectiveness of an agent at inducing one or more desired effect(s) in a living thing can be compared to the effectiveness of one, or more, other agents at inducing the same desired effect(s) in the same living thing.

It is typically easier, and more readily informative, to compare efficacy values of different agents, than to directly compare the expression patterns induced in an efficacy-related population of genes, or proteins, by the agents. For example, the efficacy value of a candidate inhibitor of a target biological response (e.g., a candidate cell division inhibitor that may be useful for inhibiting the growth of cancerous cells in a mammal) can be compared to the efficacy value of a known inhibitor of the same target, biological, response to determine whether the two efficacy values are similar. If the efficacy value of the known inhibitor is similar to the efficacy value of the candidate inhibitor, then it is inferred that the candidate inhibitor inhibits the target biological response. Again by way of example, in the context of comparing candidate inhibitors of a target biological response to determine which candidate inhibitor exerts the strongest inhibitory effect on the target biological response, the efficacy values of each candidate inhibitor are compared to each other, and it is inferred that the candidate inhibitor that has the numerically largest efficacy value exerts the strongest inhibitory effect on the target biological response.

By way of specific and more detailed example, the comparison of efficacy values may be used to identify agents that stimulate a target biological response (e.g., increase the amount of high density lipoprotein in human blood plasma). For example, a population of genes, or proteins, is identified in a living thing that yield(s) at least one expression pattern that positively correlates with the stimulation of the target biological response by at least one agent that is known to stimulate the target biological response. This is the efficacy-related gene population, or efficacy-related protein population. Living cells that include the efficacy-related gene population, or efficacy-related protein population, are contacted with a candidate agent, and the resulting expression pattern of the efficacy-related gene population, or efficacy-related protein population, is measured, and an efficacy value calculated therefrom. The efficacy value of the candidate agent is compared to the efficacy value(s) of one or more reference agent(s) that is/are known to stimulate the target biological response, and if the efficacy value of the candidate agent is sufficiently similar to the efficacy value(s) of the reference agent(s), then it is inferred that the candidate agent is a stimulant of the target biological response.

An efficacy-related population of genes, or efficacy-related protein population, can be identified, for example, by contacting a living thing (e.g., living tissue, living organ or living organism), or population of living things (e.g., population of living cells in culture), with an agent that is known to cause a target biological response. A population of genes, or proteins, is identified that yields an expression pattern that correlates (positively or negatively) with the occurrence of the target biological response in response to the agent. This population of genes, or proteins, may be used as the efficacy-related gene population, or efficacy-related protein population, respectively.

In another approach, a diseased organism may be used to identify an efficacy-related population of genes or proteins. Thus, for example, in the context of identifying chemical agents useful for ameliorating the symptoms of a target disease that affects humans, a non-human model organism (e.g., a mouse) is identified that suffers from the target disease, or that suffers from a disease that is similar to the target disease and which is a good experimental model for studying the target disease. The diseased model organism may occur naturally, or may be created by human intervention, such as by a selective breeding program, or by genetic manipulation. For example, the technique of targeted homologous recombination can be used to generate mice in which one or more genes are functionally inactivated. By choosing an appropriate gene to inactivate, the resulting mice may exhibit the symptoms of a disease that afflicts human beings, and may be a useful model system for studying the disease and for identifying candidate chemical agents useful for treating the disease.

A non-diseased organism of the same species as the diseased organism (e.g., a non-diseased mouse) is treated with an agent that is known to ameliorate the symptoms of the target disease, and the expression pattern of a representative population of genes, or proteins, from the treated organism is measured. The expression pattern of the same representative population of genes, or proteins, is measured in the diseased organism, and the expression patterns of the genes, or proteins, are compared to identify those proteins, or genes that produce transcriptional products (e.g., mRNA molecules), whose amount in the organism is affected (e.g., increased or decreased) by the agent, and which are regulated in the opposite direction in the diseased organism compared to the non-diseased organism (e.g., the level of expression of the genes is higher in a non-diseased organism than in a diseased organism, and the level of expression of the genes is increased, toward the non-diseased level, in the diseased organism in response to treatment with the agent). This population of genes, or proteins, is an efficacy-related population of genes, or an efficacy-related population of proteins, useful in the practice of the present invention for identifying agents that ameliorate the symptoms of the target disease.

Optionally, one of skill in the art may determine that a correlation (positive or negative) exists between the expression pattern of the efficacy-related gene population (or an efficacy-related population of proteins) and the amelioration of one or more symptoms of the target disease, thereby confirming the usefulness of the gene, or protein, population as an efficacy-related gene population, or efficacy-related protein population, in the practice of the methods of the present invention.

Example 1 herein describes the use of a strain of mice (referred to as db/db mice) that exhibit the symptoms of diabetes and are useful as a model experimental system for that disease. The db/db mice are used to identify an efficacy-related population of genes whose transcription is reduced in the db/db mice compared to non-diseased mice, and whose transcription is stimulated by rosiglitazone, which is a drug used to treat diabetes.

For example, an efficacy-related population of genes, or proteins, can be identified in the following manner. Living cells are contacted, in vivo or in vitro, with an amount of a first reference agent that maximally induces (or maximally inhibits) a target biological response. An example of a method for contacting living cells, cultured in vitro, with the first reference agent is addition of the first reference agent to the medium in which the living cells are cultured. Examples of methods for contacting living cells, in vivo, with the first reference agent is injection into the bloodstream, or injection into a target tissue or organ, or nasal administration of the first reference agent, or transdermal administration of the first reference agent, or use of a drug delivery device that is implanted into the body of a living subject and which gradually releases the first reference agent into the living body.

In the present example, if an efficacy-related population of genes is being sought, messenger RNA is extracted (and may or may not be purified) from the contacted cells and used as a template to synthesize cDNA or cRNA which is then labeled (e.g., with a fluorescent dye). The labeled cDNA or cRNA is then hybridized to nucleic acid molecules immobilized on a substrate (e.g., a DNA microarray). The immobilized nucleic acid molecules represent some, or all, of the genes that are expressed in the cells that were contacted with the first reference agent. The labeled cDNA or cRNA molecules that hybridize to the nucleic acid molecules immobilized on the DNA array are identified, and the level of expression of each hybridizing cDNA or cRNA is measured and compared to the level of expression of the same cDNA or cRNA species in control cells that were not contacted with the first reference agent, thereby revealing a gene expression pattern that was caused by the first reference agent. The population of genes whose expression is affected by the first reference agent can be used as the efficacy-related gene population, and an efficacy value for the first reference agent can be calculated from the levels of expression of all of the mRNAs within the efficacy-related gene population.

In the present example, if an efficacy-related population of proteins is being sought, some, or all, of the protein is extracted from the contacted cells. The identity and abundance of some or all of the proteins within the extracted protein mixture is determined by any suitable technique, such as mass spectrometry, and compared to the level of expression of the same protein species in control cells that were not contacted with the first reference agent, thereby revealing a protein expression pattern that was caused by the first reference agent. The population of proteins whose expression pattern is affected by the first reference agent can be used as the efficacy-related protein population, and an efficacy value for the first reference agent can be calculated from the levels of expression of all of the proteins within the efficacy-related protein population.

More typically, the foregoing, exemplary, procedure is repeated with one or more additional reference agents that each have the same effect as the first reference agent on the same target biological response (e.g., all the reference agents either induce or inhibit the same target biological response). The gene expression patterns, or protein expression patterns, induced by each of the reference agents are compared, and a population of genes or proteins whose expression is affected by each reference agent, and that correlates with the effect on the target biological response, is identified. The gene or protein expression patterns caused by each of the reference agents are statistically analyzed to identify the population of genes, or proteins, (within the total population of genes or proteins whose expression is affected by all the reference agents) that produces an expression pattern that most strongly correlates with the occurrence of the target biological response. This population of genes, or this population of proteins, can be used as an efficacy-related gene population, or efficacy-related protein population.

Example 1 herein describes the identification of an efficacy-related population of genes that is useful in the practice of the methods of the invention for identifying agonists and partial agonists of peroxisome proliferator-activated receptor γ (hereinafter referred to as PPARγ). The peroxisome proliferator-activated receptors are nuclear hormone receptors, activated by fatty acids and their eicosanoid metabolites, that regulate glucose and lipid homeostasis in mammals, such as human beings. The PPARγ subtype plays a central role in the regulation of adipogenesis and is the molecular target for the 2,4-thiazolidinedione class of antidiabetic drugs (e.g., rosiglitazone). See, e.g., J. L. Oberfield, et al., Proc. Nat'l Acad. Sci. U.S.A., 96:6102-6106 (1999). Undesirable side-effects caused by the 2,4-thiazolidinedione class of drugs includes heart enlargement and an increase in blood plasma volume. Thus, there is a need to identify molecules of the 2,4-thiazolidinedione class that are antidiabetic drugs, but which do not cause these undesirable side effects.

In some embodiments of the methods of the invention, the efficacy-related population of genes or proteins yields at least one efficacy-related expression pattern, in response to an agent, that correlates with the presence of at least one desired biological response caused by the agent in a living thing, wherein the at least one efficacy-related expression pattern appears before the desired biological response. Thus, for example, these embodiments of the methods of the invention are particularly useful for high-throughput screening of numerous drug candidates because it is not necessary to wait for the appearance of the desired biological response in order to identify those drug candidates that possess a defined biological activity.

Representative examples of techniques for identifying and measuring the expression of an efficacy-related population of genes: efficacy-related populations of genes are identified by measuring the amount of transcriptional expression of genes in a living thing (e.g., a living thing that has been contacted with an agent that affects a target biological response). Gene expression may be measured, for example, by extracting (and optionally purifying) mRNA from the living thing, and using the mRNA as a template to synthesize cDNA which is then labeled (e.g., with a fluorescent dye) and can be used to measure gene expression. While the following, exemplary, description is directed to embodiments of the invention in which the extracted mRNA is used as a template to synthesize cDNA, which is then labeled, it will be understood that the extracted mRNA can also be used as a template to synthesize cRNA which can then be labeled and can be used to measure gene expression.

RNA molecules useful as templates for cDNA synthesis can be isolated from any organism or part thereof, including organs, tissues, and/or individual cells. Any suitable RNA preparation can be utilized, such as total cellular RNA, or such as cytoplasmic RNA or such as an RNA preparation that is enriched for messenger RNA (mRNA), such as RNA preparations that include greater than 70%, or greater than 80%, or greater than 90%, or greater than 95%, or greater than 99% messenger RNA. Typically, RNA preparations that are enriched for messenger RNA are utilized to provide the RNA template in the practice of the methods of this aspect of the invention. Messenger RNA can be purified in accordance with any art-recognized method, such as by the use of oligo-dT columns (see, e.g., Sambrook et al., 1989, Molecular Cloning-A Laboratory Manual (2nd Ed.), Vol. 1, Chapter 7, Cold Spring Harbor Laboratory, Cold Spring Harbor, N.Y.).

Total RNA may be isolated from cells by procedures that involve breaking open the cells and, typically, denaturation of the proteins contained therein. Additional steps may be employed to remove DNA. Cell lysis may be accomplished with a nonionic detergent, followed by microcentrifugation to remove the nuclei and hence the bulk of the cellular DNA. In one embodiment, RNA is extracted from cells using guanidinium thiocyanate lysis followed by CsCl centrifugation to separate the RNA from DNA (Chirgwin et al., 1979, Biochemistry 18:5294-5299). Messenger RNA may be selected with oligo-dT cellulose (see Sambrook et al., supra). Separation of RNA from DNA can also be accomplished by organic extraction, for example, with hot phenol or phenol/chloroform/isoamyl alcohol. If desired, RNase inhibitors may be added to the lysis buffer. Likewise, for certain cell types, it may be desirable to add a protein denaturation/digestion step to the protocol.

The sample of total RNA typically includes a multiplicity of different mRNA molecules, each different mRNA molecule having a different nucleotide sequence (although there may be multiple copies of the same mRNA molecule). In a specific embodiment, the mRNA molecules in the RNA sample comprise at least 100 different nucleotide sequences. In other embodiments, the mRNA molecules of the RNA sample comprise at least 500, 1,000, 5,000, 10,000, 20,000, 30,000, 40,000, 50,000, 60,000, 70,000, 80,000, 90,000 or 100,000 different nucleotide sequences. In another specific embodiment, the RNA sample is a mammalian RNA sample, the mRNA molecules of the mammalian RNA sample comprising about 20,000 to 30,000 different nucleotide sequences, or comprising substantially all of the different mRNA sequences that are expressed in the cell(s) from which the mRNA was extracted.

In the context of the present example, cDNA molecules are synthesized that are complementary to the RNA template molecules. Each cDNA molecule is preferably sufficiently long (e.g., at least 50 nucleotides in length) to subsequently serve as a specific probe for the mRNA template from which it was synthesized, or to serve as a specific probe for a DNA sequence that is identical to the sequence of the mRNA template from which the cDNA molecule was synthesized. Individual DNA molecules can be complementary to a whole RNA template molecule, or to a portion thereof. Thus, a population of cDNA molecules is synthesized that includes individual DNA molecules that are each complementary to all, or to a portion, of a template RNA molecule. Typically, at least a portion of the complementary sequence of at least 95% (more typically at least 99%) of the template RNA molecules are represented in the population of cDNA molecules.

Any reverse transcriptase molecule can be utilized to synthesize the cDNA molecules, such as reverse transcriptase molecules derived from Moloney murine leukemia virus (MMLV-RT), avian myeloblastosis virus (AMV-RT), bovine leukemia virus (BLV-RT), Rous sarcoma virus (RSV) and human immunodeficiency virus (HIV-RT). A reverse transcriptase lacking RNaseH activity (e.g., SUPERSCRIPT II™ sold by Stratagene, La Jolla, Calif.) has the advantage that, in the absence of an RNaseH activity, synthesis of second strand cDNA molecules does not occur during synthesis of first strand cDNA molecules. The reverse transcriptase molecule should also preferably be thermostable so that the cDNA synthesis reaction can be conducted at as high a temperature as possible, while still permitting hybridization of any required primer(s) to the RNA template molecules.

The synthesis of the cDNA molecules can be primed using any suitable primer, typically an oligonucleotide in the range of ten to 60 bases in length. Oligonucleotides that are useful for priming the synthesis of the cDNA molecules can hybridize to any portion of the RNA template molecules, including the oligo-dT tail. In some embodiments, the synthesis of the cDNA molecules is primed using a mixture of primers, such as a mixture of primers having random nucleotide sequences. Typically, for oligonucleotide molecules less than 100 bases in length, hybridization conditions are 5° C. to 10° C. below the homoduplex melting temperature (Tm); see generally, Sambrook et al. Molecular Cloning: A Laboratory Manual, 2nd ed., Cold Spring Harbor Press, 1987; Ausubel et al., Current Protocols in Molecular Biology, Greene Publishing, 1987).

A primer for priming cDNA synthesis can be prepared by any suitable method, such as phosphotriester and phosphodiester methods of synthesis, or automated embodiments thereof. It is also possible to use a primer that has been isolated from a biological source, such as a restriction endonuclease digest. An oligonucleotide primer can be DNA, RNA, chimeric mixtures or derivatives or modified versions thereof, so long as it is still capable of priming the desired reaction. The oligonucleotide primer can be modified at the base moiety, sugar moiety, or phosphate backbone, and may include other appending groups or labels, so long as it is still capable of priming cDNA synthesis.

An oligonucleotide primer for priming cDNA synthesis can be derived by cleavage of a larger nucleic acid fragment using non-specific nucleic acid cleaving chemicals or enzymes or site-specific restriction endonucleases; or by synthesis by standard methods known in the art, e.g., by use of an automated DNA synthesizer (such as are commercially available from Biosearch, Applied Biosystems, etc.) and standard phosphoramidite chemistry. As examples, phosphorothioate oligonucleotides may be synthesized by the method of Stein et al. (Nucl. Acids Res. 16:3209-3221, 1988), methylphosphonate oligonucleotides can be prepared by use of controlled pore glass polymer supports (Sarin et al., 1988, Proc. Natl. Acad. Sci. U.S.A. 85:7448-7451).

Once the desired oligonucleotide is synthesized, it is cleaved from the solid support on which it was synthesized and treated, by methods known in the art, to remove any protecting groups present. The oligonucleotide may then be purified by any method known in the art, including extraction and gel purification. The concentration and purity of the oligonucleotide may be determined, for example, by examining the oligonucleotide that has been separated on an acrylamide gel, or by measuring the optical density at 260 nm in a spectrophotometer.

After cDNA synthesis is complete, the RNA template molecules can be hydrolyzed, and all, or substantially all (typically more than 99%), of the primers can be removed. Hydrolysis of the RNA template can be achieved, for example, by alkalinization of the solution containing the RNA template (e.g., by addition of an aliquot of a concentrated sodium hydroxide solution). The primers can be removed, for example, by applying the solution containing the RNA template molecules, cDNA molecules, and the primers, to a column that separates nucleic acid molecules on the basis of size. The purified, cDNA molecules, can then, for example, be precipitated and redissolved in a suitable buffer.

The cDNA molecules are typically labeled to facilitate the detection of the cDNA molecules when they are used as a probe in a hybridization experiment, such as a probe used to screen a DNA microarray, to identify an efficacy-related population of genes. The cDNA molecules can be labeled with any useful label, such as a radioactive atom (e.g., ³²P), but typically the cDNA molecules are labeled with a dye. Examples of suitable dyes include fluorophores and chemiluminescers.

By way of example, cDNA molecules can be coupled to dye molecules via aminoallyl linkages by incorporating allylamine-derivatized nucleotides (e.g., allylamine-dATP, allylamine-dCTP, allylamine-dGTP, and/or allylamine-dTTP) into the cDNA molecules during synthesis of the cDNA molecules. The allylamine-derivatized nucleotide(s) can then be coupled, via an aminoallyl linkage, to N-hydroxysuccinimide ester derivatives (NHS derivatives) of dyes (e.g., Cy-NHS, Cy3-NHS and/or Cy5-NHS). Again by way of example, in another embodiment, dye-labeled nucleotides may be incorporated into the cDNA molecules during synthesis of the cDNA molecules, which labels the cDNA molecules directly.

It is also possible to include a spacer (usually 5-16 carbon atoms long) between the dye and the nucleotide, which may improve enzymatic incorporation of the modified nucleotides during synthesis of the cDNA molecules.

In the context of the present example, the labeled cDNA is hybridized to a DNA array that includes hundreds, or thousands, of identified nucleic acid molecules (e.g., cDNA molecules) that correspond to genes that are expressed in the type of cells wherein gene expression is being analyzed. Typically, hybridization conditions used to hybridize the labeled cDNA to a DNA array are no more than 25° C. to 30° C. (for example, 10° C.) below the melting temperature (Tm) of the native duplex of the cDNA that has the lowest melting temperature (see generally, Sambrook et al. Molecular Cloning: A Laboratory Manual, 2nd ed., Cold Spring Harbor Press, 1987; Ausubel et al., Current Protocols in Molecular Biology, Greene Publishing, 1987). Tm for nucleic acid molecules greater than about 100 bases can be calculated by the formula Tₘ=81.5+0.41%(G+C)−log(Na+). For oligonucleotide molecules less than 100 bases in length, exemplary hybridization conditions are 5° to 10° C. below Tm.

Preparation of microarrays. Nucleic acid molecules can be immobilized on a solid substrate by any art-recognized means. For example, nucleic acid molecules (such as DNA or RNA molecules) can be immobilized to nitrocellulose, or to a synthetic membrane capable of binding nucleic acid molecules, or to a nucleic acid microarray, such as a DNA microarray. A DNA microarray, or chip, is a microscopic array of DNA fragments, such as synthetic oligonucleotides, disposed in a defined pattern on a solid support, wherein they are amenable to analysis by standard hybridization methods (see, Schena, BioEssays 18: 427, 1996).

The DNA in a microarray may be derived, for example, from genomic or cDNA libraries, from fully sequenced clones, or from partially sequenced cDNAs known as expressed sequence tags (ESTs). Methods for obtaining such DNA molecules are generally known in the art (see, e.g., Ausubel et al., eds., 1994, Current Protocols in Molecular Biology, Vol. 2, Current Protocols Publishing, New York). Again by way of example, oligonucleotides may be synthesized by conventional methods, such as the methods described herein.

Microarrays can be made in a number of ways, of which several are described below. However produced, microarrays preferably share certain characteristics. The arrays are preferably reproducible, allowing multiple copies of a given array to be produced and easily compared with each other. Preferably the microarrays are small, usually smaller than 5 cm², and they are made from materials that are stable under nucleic acid hybridization conditions. A given binding site or unique set of binding sites in the microarray should specifically bind the product of a single gene (or a nucleic acid molecule that represents the product of a single gene, such as a cDNA molecule that is complementary to all, or to part, of an mRNA molecule). Although there may be more than one physical binding site (hereinafter “site”) per specific gene product, for the sake of clarity the discussion below will assume that there is a single site.

In one embodiment, the microarray is an array of polynucleotide probes, the array comprising a support with at least one surface and typically at least 100 different polynucleotide probes, each different polynucleotide probe comprising a different nucleotide sequence and being attached to the surface of the support in a different location on the surface. For example, the nucleotide sequence of each of the different polynucleotide probes can be in the range of 40 to 80 nucleotides in length. For example, the nucleotide sequence of each of the different polynucleotide probes can be in the range of 50 to 70 nucleotides in length. For example, the nucleotide sequence of each of the different polynucleotide probes can be in the range of 50 to 60 nucleotides in length. In specific embodiments, the array comprises polynucleotide probes of at least 2,000, 4,000, 10,000, 15,000, 20,000, 50,000, 80,000, or 100,000 different nucleotide sequences.

Thus, the array can include polynucleotide probes for most, or all, genes expressed in a cell, tissue, organ or organism. In a specific embodiment, the cell or organism is a mammalian cell or organism. In another specific embodiment, the cell or organism is a human cell or organism. In specific embodiments, the nucleotide sequences of the different polynucleotide probes of the array are specific for at least 50%, at least 75%, at least 80%, at least 85%, at least 90%, at least 95%, or at least 99% of the genes in the genome of the cell or organism. Most preferably, the nucleotide sequences of the different polynucleotide probes of the array are specific for all of the genes in the genome of the cell or organism. In specific embodiments, the polynucleotide probes of the array hybridize specifically and distinguishably to at least 10,000, to at least 20,000, to at least 50,000, to at least 80,000, or to at least 100,000 different polynucleotide sequences. In other specific embodiments, the polynucleotide probes of the array hybridize specifically and distinguishably to at least 90%, at least 95%, or at least 99% of the genes or gene transcripts of the genome of a cell or organism. Most preferably, the polynucleotide probes of the array hybridize specifically and distinguishably to the genes or gene transcripts of the entire genome of a cell or organism.

In specific embodiments, the array has at least 100, at least 250, at least 1,000, or at least 2,500 probes per 1 cm², preferably all or at least 25% or 50% of which are different from each other. In another embodiment, the array is a positionally addressable array (in that the sequence of the polynucleotide probe at each position is known). In another embodiment, the nucleotide sequence of each polynucleotide probe in the array is a DNA sequence. In another embodiment, the DNA sequence is a single-stranded DNA sequence. The DNA sequence may be, e.g., a cDNA sequence, or a synthetic sequence.

When a cDNA molecule that corresponds to an mRNA of a cell is made and hybridized to a microarray under suitable hybridization conditions, the level of hybridization to the site in the array corresponding to any particular gene will reflect the prevalence in the cell of mRNA transcribed from that gene. For example, when detectably labeled (e.g., with a fluorophore) DNA complementary to the total cellular mRNA is hybridized to a microarray, the site on the array corresponding to a gene (i.e., capable of specifically binding the product of the gene) that is not transcribed in the cell will have little or no signal (e.g., fluorescent signal), and a gene for which the encoded mRNA is prevalent will have a relatively strong signal.

In some embodiments, cDNA molecule populations prepared from RNA from two different cell populations, or tissues, or organs, or whole organisms, are hybridized to the binding sites of the array. A single array can be used to simultaneously screen more than one cDNA sample. For example, in the context of the present invention, a single array can be used to simultaneously screen a cDNA sample prepared from a living thing that has been contacted with an agent (e.g., candidate partial agonist of PPARγ), and the same type of living thing that has not been contacted with the agent. The cDNA molecules in the two samples are differently labeled so that they can be distinguished. In one embodiment, for example, cDNA molecules from a cell population treated with a drug is synthesized using a fluorescein-labeled NTP, and cDNA molecules from a control cell population, not treated with the drug, is synthesized using a rhodamine-labeled NTP. When the two populations of cDNA molecules are mixed and hybridized to the DNA array, the relative intensity of signal from each population of cDNA molecules is determined for each site on the array, and any relative difference in abundance of a particular mRNA detected.

In this representative example, the cDNA molecule population from the drug-treated cells will fluoresce green when the fluorophore is stimulated, and the cDNA molecule population from the untreated cells will fluoresce red. As a result, when the drug treatment has no effect, either directly or indirectly, on the relative abundance of a particular mRNA in a cell, the mRNA will be equally prevalent in treated and untreated cells and red-labeled and green-labeled cDNA molecules will be equally prevalent. When hybridized to the DNA array, the binding site(s) for that species of RNA will emit wavelengths characteristic of both fluorophores (and appear brown in combination). In contrast, when the drug-exposed cell is treated with a drug that, directly or indirectly, increases the prevalence of the mRNA in the cell, the ratio of green to red fluorescence will increase. When the drug decreases the mRNA prevalence, the ratio will decrease.

The use of a two-color fluorescence labeling and detection scheme to define alterations in gene expression has been described, e.g., in Schena et al., 1995, Science 270:467-470, which is incorporated by reference in its entirety for all purposes. An advantage of using cDNA molecules labeled with two different fluorophores is that a direct and internally controlled comparison of the mRNA levels corresponding to each arrayed gene in two cell states can be made, and variations due to minor differences in experimental conditions (e.g., hybridization conditions) will not affect subsequent analyses. However, it will be recognized that it is also possible to use cDNA molecules from a single cell, and compare, for example, the absolute amount of a particular mRNA in, e.g., a drug-treated or an untreated cell.

Exemplary microarrays and methods for their manufacture and use are set forth in T. R. Hughes et al., Nature Biotechnology 19: 342-347 (April 2001), which publication is incorporated herein by reference.

Preparation of nucleic acid molecules for immobilization on microarrays. As noted above, the “binding site” to which a particular, cognate, nucleic acid molecule specifically hybridizes is usually a nucleic acid, or nucleic acid analogue, attached at that binding site. In one embodiment, the binding sites of the microarray are DNA polynucleotides corresponding to at least a portion of some or all genes in an organism's genome. These DNAs can be obtained by, for example, polymerase chain reaction (PCR) amplification of gene segments from genomic DNA, cDNA (e.g., by reverse transcription or RT-PCR), or cloned sequences. Nucleic acid amplification primers are chosen, based on the known sequence of the genes or cDNA, that result in amplification of unique fragments (i.e., fragments that typically do not share more than 10 bases of contiguous identical sequence with any other fragment on the microarray). Computer programs are useful in the design of primers with the required specificity and optimal amplification properties. See, e.g., Oligo version 5.0 (National Biosciences). Typically each gene fragment on the microarray will be between about 50 bp and about 2000 bp, more typically between about 100 bp and about 1000 bp, and usually between about 300 bp and about 800 bp in length.

Nucleic acid amplification methods are well known and are described, for example, in Innis et al., eds., 1990, PCR Protocols: A Guide to Methods and Applications, Academic Press Inc., San Diego, Calif., which is incorporated by reference in its entirety for all purposes. Computer controlled robotic systems are useful for isolating and amplifying nucleic acids.

An alternative means for generating the nucleic acid molecules for the microarray is by synthesis of synthetic polynucleotides or oligonucleotides, e.g., using N-phosphonate or phosphoramidite chemistries (e.g., Froehler et al., 1986, Nucleic Acid Res 14:5399-5407). Synthetic sequences are typically between about 15 and about 100 bases in length, such as between about 20 and about 50 bases.

In some embodiments, synthetic nucleic acids include non-natural bases, e.g., inosine. Where the particular base in a given sequence is unknown or is polymorphic, a universal base, such as inosine or 5-nitroindole, may be substituted. Additionally, it is possible to vary the charge on the phosphate backbone of the oligonucleotide, for example, by thiolation or methylation, or even to use a peptide rather than a phosphate backbone. The making of such modifications is within the skill of one trained in the art.

As noted above, nucleic acid analogues may be used as binding sites for hybridization. An example of a suitable nucleic acid analogue is peptide nucleic acid (see, e.g., Egholm et al., 1993, Nature 365:566-568; see also U.S. Pat. No. 5,539,083).

In another embodiment, the binding (hybridization) sites are made from plasmid or phage clones of genes, cDNAs (e.g., expressed sequence tags), or inserts therefrom (Nguyen et al., 1995, Genomics 29:207-209). In yet another embodiment, the polynucleotide of the binding sites is RNA.

Attaching nucleic acids to the solid support. The nucleic acids, or analogues, are attached to a solid support, which may be made, for example, from glass, silicon, plastic (e.g., polypropylene, nylon, polyester), polyacrylamide, nitrocellulose, cellulose acetate or other materials. In general, non-porous supports, and glass in particular, are preferred. The solid support may also be treated in such a way as to enhance binding of oligonucleotides thereto, or to reduce non-specific binding of unwanted substances thereto. For example, a glass support may be treated with polylysine or silane to facilitate attachment of oligonucleotides to the slide.

Methods of immobilizing DNA on the solid support may include direct touch, micropipetting (see, e.g., Yershov et al., Proc. Natl. Acad. Sci. USA 93(10):4913-4918 (1996)), or the use of controlled electric fields to direct a given oligonucleotide to a specific spot in the array. Oligonucleotides are typically immobilized at a density of 100 to 10,000 oligonucleotides per cm², such as at a density of about 1000 oligonucleotides per cm².

A preferred method for attaching the nucleic acids to a surface is by printing on glass plates, as is described generally by Schena et al., 1995, Science 270:467-470. This method is especially useful for preparing microarrays of cDNA. (See also DeRisi et al., 1996, Nature Genetics 14:457-460; Shalon et al., 1996, Genome Res. 6:639-645; and Schena et al., Proc. Natl. Acad. Sci. USA 93(20):10614-19, 1996.)

In an alternative to immobilizing pre-fabricated oligonucleotides onto a solid support, it is possible to synthesize oligonucleotides directly on the support (see, e.g., Maskos et al., Nucl. Acids Res. 21:2269-70, 1993; Lipshutz et al., 1999, Nat. Genet. 21(1 Suppl):20-4). Methods of synthesizing oligonucleotides directly on a solid support include photolithography (see McGall et al., Proc. Natl. Acad. Sci. (USA) 93:13555-60, 1996) and piezoelectric printing (Lipshutz et al., 1999, Nat. Genet. 21(1 Suppl):20-4).

A high-density oligonucleotide array may be employed. Techniques are known for producing arrays containing thousands of oligonucleotides complementary to defined sequences, at defined locations on a surface using photolithographic techniques for synthesis in situ (see, Pease et al., 1994, Proc. Natl. Acad. Sci. USA 91:5022-5026; Lockhart et al., 1996, Nature Biotechnol. 14:1675-80) or other methods for rapid synthesis and deposition of defined oligonucleotides (Lipshutz et al., 1999, Nat. Genet. 21(1 Suppl):20-4.).

In some embodiments, microarrays are manufactured by means of an ink jet printing device for oligonucleotide synthesis, e.g., using the methods and systems described by Blanchard in International Patent Publication No. WO 98/41531, published Sep. 24, 1998; Blanchard et al., 1996, Biosensors and Bioeletronics 11:687-690; Blanchard, 1998, in Synthetic DNA Arrays in Genetic Engineering, Vol. 20, J. K. Setlow, Ed., Plenum Press, New York at pages 111-123; U.S. Pat. No. 6,028,189 to Blanchard. Specifically, the oligonucleotide probes in such microarrays are preferably synthesized in arrays, e.g., on a glass slide, by serially depositing individual nucleotide bases in “microdroplets” of a high surface tension solvent such as propylene carbonate. The microdroplets have small volumes (e.g., 100 pL or less, more preferably 50 pL or less) and are separated from each other on the microarray (e.g., by hydrophobic domains) to form circular surface tension wells which define the locations of the array elements (i.e., the different probes).

Other methods for making microarrays, e.g., by masking (Maskos and Southern, 1992, Nuc. Acids Res. 20:1679-1684), may also be used. In principle, any type of array, for example dot blots on a nylon hybridization membrane (see Sambrook et al., 1989, Molecular Cloning—A Laboratory Manual (2nd Ed.), Vols. 1-3, Cold Spring Harbor Laboratory, Cold Spring Harbor, N.Y.), could be used, although, as will be recognized by those of skill in the art, very small arrays are typically preferred because hybridization volumes will be smaller.

Signal detection and data analysis. When fluorescently labeled probes are used, the fluorescence emissions at each site of an array can be detected by scanning confocal laser microscopy. In one embodiment, a separate scan, using the appropriate excitation line, is carried out for each of the two fluorophores used. Alternatively, a laser can be used that allows simultaneous specimen illumination at wavelengths specific to the two fluorophores and emissions from the two fluorophores can be analyzed simultaneously (see Shalon et al., 1996, Genome Research 6:639-645, which is incorporated by reference in its entirety for all purposes). In one embodiment, the arrays are scanned with a laser fluorescent scanner with a computer controlled X-Y stage and a microscope objective. Sequential excitation of the two fluorophores is achieved with a multi-line, mixed gas laser and the emitted light is split by wavelength and detected with two photomultiplier tubes. Fluorescence laser scanning devices are described in Shalon et al., 1996, Genome Res. 6:639-645 and in other references cited herein. Alternatively, the fiber-optic bundle described by Ferguson et al., 1996, Nature Biotechnol. 14:1681-1684, may be used to monitor mRNA abundance levels at a large number of sites simultaneously.

Signals are recorded and may be analyzed by computer, e.g., using a 12 bit analog to digital board. In some embodiments the scanned image is despeckled using a graphics program (e.g., Hijaak Graphics Suite) and then analyzed using an image gridding program that creates a spreadsheet of the average hybridization at each wavelength at each site. If necessary, an experimentally determined correction for “cross talk” (or overlap) between the channels for the two fluors may be made. For any particular hybridization site on the transcript array, a ratio of the emission of the two fluorophores can be calculated. The ratio is independent of the absolute expression level of the cognate gene, but is useful for genes whose expression is significantly modulated by drug administration.

The relative abundance of an mRNA in two biological samples is scored as a perturbation and its magnitude determined (i.e., the abundance is different in the two sources of mRNA tested), or as not perturbed (i.e., the relative abundance is the same). Preferably, in addition to identifying a perturbation as positive or negative, it is advantageous to determine the magnitude of the perturbation. This can be carried out, as noted above, by calculating the ratio of the emission of the two fluorophores used for differential labeling, or by analogous methods that will be readily apparent to those of skill in the art.

By way of example, two samples, each labeled with a different fluor, are hybridized simultaneously to permit differential expression measurements. If neither sample hybridizes to a given spot in the array, no fluorescence will be seen. If only one hybridizes to a given spot, the color of the resulting fluorescence will correspond to that of the fluor used to label the hybridizing sample (for example, green if the sample was labeled with Cy3, or red, if the sample was labeled with Cy5). If both samples hybridize to the same spot, an intermediate color is produced (for example, yellow if the samples were labeled with fluorescein and rhodamine). Then, applying methods of pattern recognition and data analysis known in the art, it is possible to quantify differences in gene expression between the samples. Methods of pattern recognition and data analysis are described in e.g., International Publication WO 00/24936, which is incorporated by reference herein.

Measurement of Expression Pattern of an Efficacy-Related Population of Proteins: In the practice of some embodiments of the present invention, the expression pattern of an efficacy-related population of proteins in a living thing is measured. Any useful method for measuring protein expression patterns can be used. Typically all, or substantially all, proteins are extracted from a living thing, or a portion thereof. The living thing is typically treated to disrupt cells, for example by homogenizing the cellular material in a blender, or by grinding (in the presence of acid-washed, siliconized, sand if desired) the cellular material with a mortar and pestle, or by subjecting the cellular material to osmotic stress that lyses the cells. Cell disruption may be carried out in the presence of a buffer that maintains the released contents of the disrupted cells at a desired pH, such as the physiological pH of the cells. The buffer may optionally contain inhibitors of endogenous proteases. Physical disruption of the cells can be conducted in the presence of chemical agents (e.g., detergents) that promote the release of proteins.

The cellular material may be treated in a manner that does not disrupt a significant proportion of cells, but which removes proteins from the surface of the cellular material, and/or from the interstices between cells. For example, cellular material can be soaked in a liquid buffer, or, in the case of plant material, can be subjected to a vacuum, in order to remove proteins located in the intercellular spaces and/or in the plant cell wall. If the cellular material is a microorganism, proteins can be extracted from the microorganism culture medium.

It may be desirable to include one or more protease inhibitors in the protein extraction buffer. Representative examples of protease inhibitors include: serine protease inhibitors (such as phenylmethylsulfonyl fluoride (PMSF), benzamide, benzamidine HCl, ε-Amino-n-caproic acid and aprotinin (Trasylol)); cysteine protease inhibitors, such as sodium p-hydroxymercuribenzoate; competitive protease inhibitors, such as antipain and leupeptin; covalent protease inhibitors, such as iodoacetate and N-ethylmaleimide; aspartate (acidic) protease inhibitors, such as pepstatin and diazoacetylnorleucine methyl ester (DAN); metalloprotease inhibitors, such as EGTA [ethylene glycol bis(β-aminoethyl ether) N,N,N′N′-tetraacetic acid], and the chelator 1, 10-phenanthroline.

The mixture of released proteins may, or may not, be treated to completely or partially purify some of the proteins for further analysis, and/or to remove non-protein contaminants (e.g., carbohydrates and lipids). In some embodiments, the complete mixture of released proteins is analyzed to determine the amount and/or identity of some or all of the proteins. For example, the protein mixture may be applied to a substrate bearing antibody molecules that specifically bind to one or more proteins in the mixture. The unbound proteins are removed (e.g., washed away with a buffer solution), and the amount of bound protein(s) is measured. Representative techniques for measuring the amount of protein using antibodies are described in Harlow and Lane, 1988, Antibodies: A Laboratory Manual, Cold Spring Harbor, N.Y., and include such techniques as the ELISA assay. Moreover, protein microarrays can be used to simultaneously measure the amount of a multiplicity of proteins. A surface of the microarray bears protein binding agents, such as monoclonal antibodies specific to a plurality of protein species. Preferably, antibodies are present for a substantial fraction of the encoded proteins, or at least for those proteins whose amount is to be measured. Methods for making monoclonal antibodies are well known (see, e.g., Harlow and Lane, 1988, Antibodies: A Laboratory Manual, Cold Spring Harbor, N.Y.). Protein binding agents are not restricted to monoclonal antibodies, and can be, for example, scFv/Fab diabodies, affibodies, and aptamers. Protein microarrays are generally described by M. F. Templin et al., Protein Microarray Technology, Trends in Biotechnology, 20(4):160-166(2002). Representative examples of protein microarrays are described by H. Zhu et al., Global Analysis of Protein Activities Using Proteome Chips, Science, 293:2102-2105 (2001); and G. MacBeath and S. L. Schreiber, Printing Proteins as Microarrays for High-Throughput Function Determination, Science, 289:1760-1763 (2000).

In some embodiments, the released protein is treated to completely or partially purify some of the proteins for further analysis, and/or to remove non-protein contaminants. Any useful purification technique, or combination of techniques, can be used. For example, a solution containing extracted proteins can be treated to selectively precipitate certain proteins, such as by dissolving ammonium sulfate in the solution, or by adding trichloroacetic acid. The precipitated material can be separated from the unprecipitated material, for example by centrifugation, or by filtration. The precipitated material can be further fractionated if so desired.

By way of example, a number of different neutral or slightly acidic salts have been used to solubilize, precipitate, or fractionate proteins in a differential manner. These include NaCl, Na₂SO₄, MgSO₄ and NH₄(SO₄)₂. Ammonium sulfate is a commonly used precipitant for salting proteins out of solution. The solution to be treated with ammonium sulfate may first be clarified by centrifugation. The solution should be in a buffer at neutral pH unless there is a reason to conduct the precipitation at another pH; in most cases the buffer will have ionic strength close to physiological. Precipitation is usually performed at 0-4° C. (to reduce the rate of proteolysis caused by proteases in the solution), and all solutions should be precooled to that temperature range.

Representative examples of other art-recognized techniques for purifying, or partially purifying, proteins from a living thing are exclusion chromatography, ion-exchange chromatography, hydrophobic interaction chromatography, reversed-phase chromatography and immobilized metal affinity chromatography.

Hydrophobic interaction chromatography and reversed-phase chromatography are two separation methods based on the interactions between the hydrophobic moieties of a sample and an insoluble, immobilized hydrophobic group present on the chromatography matrix. In hydrophobic interaction chromatography the matrix is hydrophilic and is substituted with short-chain phenyl or octyl nonpolar groups. The mobile phase is usually an aqueous salt solution. In reversed phase chromatography the matrix is silica that has been substituted with longer n-alkyl chains, usually C₈ (octylsilyl) or C₁₈ (octadecylsilyl). The matrix is less polar than the mobile phase. The mobile phase is usually a mixture of water and a less polar organic modifier.

Separations on hydrophobic interaction chromatography matrices are usually done in aqueous salt solutions, which generally are nondenaturing conditions. Samples are loaded onto the matrix in a high-salt buffer and elution is by a descending salt gradient. Separations on reversed-phase media are usually done in mixtures of aqueous and organic solvents, which are often denaturing conditions. In the case of protein purification, hydrophobic interaction chromatography depends on surface hydrophobic groups and is usually carried out under conditions which maintain the integrity of the protein molecule. Reversed-phase chromatography depends on the native hydrophobicity of the protein and is carried out under conditions which expose nearly all hydrophobic groups to the matrix, i.e., denaturing conditions.

Ion-exchange chromatography is designed specifically for the separation of ionic or ionizable compounds. The stationary phase (column matrix material) carries ionizable functional groups, fixed by chemical bonding to the stationary phase. These fixed charges carry a counterion of opposite sign. This counterion is not fixed and can be displaced. Ion-exchange chromatography is named on the basis of the sign of the displaceable charges. Thus, in anion ion-exchange chromatography the fixed charges are positive and in cation ion-exchange chromatography the fixed charges are negative.

Retention of a molecule on an ion-exchange chromatography column involves an electrostatic interaction between the fixed charges and those of the molecule, binding involves replacement of the nonfixed ions by the molecule. Elution, in turn, involves displacement of the molecule from the fixed charges by a new counterion with a greater affinity for the fixed charges than the molecule, and which then becomes the new, nonfixed ion.

The ability of counterions (salts) to displace molecules bound to fixed charges is a function of the difference in affinities between the fixed charges and the nonfixed charges of both the molecule and the salt. Affinities in turn are affected by several variables, including the magnitude of the net charge of the molecule and the concentration and type of salt used for displacement.

Solid-phase packings used in ion-exchange chromatography include cellulose, dextrans, agarose, and polystyrene. The exchange groups used include DEAE (diethylaminoethyl), a weak base, that will have a net positive charge when ionized and will therefore bind and exchange anions; and CM (carboxymethyl), a weak acid, with a negative charge when ionized that will bind and exchange cations. Another form of weak anion exchanger contains the PEI (polyethyleneimine) functional group. This material, most usually found on thin layer sheets, is useful for binding proteins at pH values above their pI. The polystyrene matrix can be obtained with quaternary ammonium functional groups for strong base anion exchange or with sulfonic acid functional groups for strong acid cation exchange. Intermediate and weak ion-exchange materials are also available. Ion-exchange chromatography need not be performed using a column, and can be performed as batch ion-exchange chromatography with the slurry of the stationary phase in a vessel such as a beaker.

Gel filtration is performed using porous beads as the chromatographic support. A column constructed from such beads will have two measurable liquid volumes, the external volume, consisting of the liquid between the beads, and the internal volume, consisting of the liquid within the pores of the beads. Large molecules will equilibrate only with the external volume while small molecules will equilibrate with both the external and internal volumes. A mixture of molecules (such as proteins) is applied in a discrete volume or zone at the top of a gel filtration column and allowed to percolate through the column. The large molecules are excluded from the internal volume and therefore emerge first from the column while the smaller molecules, which can access the internal volume, emerge later. The volume of a conventional matrix used for protein purification is typically 30 to 100 times the volume of the sample to be fractionated. The absorbance of the column effluent can be continuously monitored at a desired wavelength using a flow monitor.

A technique that can be applied to the purification of proteins is High Performance Liquid Chromatography (HPLC). HPLC is an advancement in both the operational theory and fabrication of traditional chromatographic systems. HPLC systems for the separation of biological macromolecules vary from the traditional column chromatographic systems in three ways; (1) the column packing materials are of much greater mechanical strength, (2) the particle size of the column packing materials has been decreased 5- to 10-fold to enhance adsorption-desorption kinetics and diminish bandspreading, and (3) the columns are operated at 10-60 times higher mobile-phase velocity. Thus, by way of non-limiting example, HPLC can utilize exclusion chromatography, ion-exchange chromatography, hydrophobic interaction chromatography, reversed-phase chromatography and immobilized metal affinity chromatography.

An exemplary technique that is useful for measuring the amounts of individual proteins in a mixture of proteins is two dimensional gel electrophoresis. This technique typically involves isoelectric focussing of a protein mixture along a first dimension, followed by SDS-PAGE of the focussed proteins along a second dimension (see, e.g., Hames et al., 1990, Gel Electrophoresis of Proteins: A Practical Approach, IRL Press, New York; Shevchenko et al., 1996, Proc. Nat'l Acad. Sci. U.S.A. 93:1440-1445; Sagliocco et al., 1996, Yeast 12:1519-1533; Lander, 1996, Science 274:536-539; and Beaumont et al., Life Science News, 7, 2001, Amersham Pharmacia Biotech. The resulting series of protein “spots” on the second dimension SDS-PAGE gel can be measured to reveal the amount of one or more specific proteins in the mixture. The identity of the measured proteins may, or may not, be known; it is only necessary to be able to identify and measure specific protein “spots” on the second dimension gel. Numerous techniques are available to measure the amount of protein in a “spot” on the second dimension gel. For example, the gel can be stained with a reagent that binds to proteins and yields a visible protein “spot” (e.g., Coomassie blue dye, or staining with silver nitrate), and the density of the stained spot can be measured. Again by way of example, all, or most, proteins in a mixture can be measured with a fluorescent reagent before electrophoretic separation, and the amount of fluorescence in some, or all, of the resolved protein “spots” can be measured (see, e.g., Beaumont et al., Life Science News, 7, 2001, Amersham Pharmacia Biotech).

Again by way of example, any HPLC technique (e.g., exclusion chromatography, ion-exchange chromatography, hydrophobic interaction chromatography, reversed-phase chromatography and immobilized metal affinity chromatography) can be used to separate proteins in a mixture, and the separated proteins can thereafter be directed to a detector (e.g., spectrophotometer) that detects and measures the amount of individual proteins.

In some embodiments of the invention it is desirable to both identify and measure the amount of specific proteins. A technique that is useful in these embodiments of the invention is mass spectrometry, in particular the techniques of electrospray ionization mass spectrometry (ESI-MS) and matrix-assisted laser desorption/ionization mass spectrometry (MALDI-MS), although it is understood that mass spectrometry can be used only to measure the amounts of proteins without also identifying (by function and/or sequence) the proteins. These techniques overcame the problem of generating ions from large, non-volatile, analytes, such as proteins, without significant analyte fragmentation (see, e.g., R. Aebersold and D. R. Goodlett, Mass Spectrometry in Proteomics, Chemical Reviews, 102(2): 269-296 (2001)).

Thus, for example, proteins can be extracted from cells of a living thing and individual proteins purified therefrom using, for example, any of the art-recognized purification techniques described herein (e.g., HPLC). The purified proteins are subjected to enzymatic degradation using a protein-degrading agent (e.g., an enzyme, such as trypsin) that cleaves proteins at specific amino acid sequences. The resulting protein fragments are subjected to mass spectrometry. If the sequence of the complete genome (or at least the sequence of part of the genome) of the living thing from which the proteins were isolated is known, then computer algorithms are available that can compare the observed protein fragments to the protein fragments that are predicted to exist by cleaving the proteins encoded by the genome with the agent used to cleave the extracted proteins. Thus, the identity, and the amount, of the proteins from which the observed fragments are derived can be determined.

Again by way of example, the use of isotope-coded affinity tags in conjunction with mass spectrometry is a technique that is adapted to permit comparison of the identities and amounts of proteins expressed in different samples of the same type of living thing subjected to different treatments (e.g., the same type of living tissue cultured, in vitro, in the presence or absence of a candidate drug)(see, e.g., S. P. Gygi et al., Quantitative Analysis of Complex Protein Mixtures Using Isotope-Coded Affinity Tags (ICATs), Nature Biotechnology, 17:994-999(1999)). In an exemplary embodiment of this method, two different samples of the same type of living thing are subjected to two different treatments (treatment 1 and treatment 2). Proteins are extracted from the treated living things and are labeled (via cysteine residues) with an ICAT reagent that includes (1) a thiol-specific reactive group, (2) a linker that can include eight deuteriums (yielding a heavy ICAT reagent) or no deuteriums (yielding a light ICAT reagent), and (3) a biotin molecule. Thus, for example, the proteins from treatment 1 may be labeled with the heavy ICAT reagent, and proteins from treatment 2 may be labelled with the light ICAT reagent. The labeled proteins from treatment 1 and treatment 2 are combined and enzymatically cleaved to generate peptide fragments. The tagged (cysteine-containing) fragments are isolated by avidin affinity chromatography (that binds the biotin moiety of the ICAT reagent). The isolated peptides are then separated by mass spectrometry. The quantity and identity of the peptides (and the proteins from which they are derived) may be determined. The method is also applicable to proteins that do not include cysteines by using ICAT reagents that label other amino acids.

Comparison of Gene Expression Levels: Art-recognized statistical techniques can be used to compare the levels of expression of individual genes, or proteins, to identify genes, or proteins, which exhibit significantly different expression levels in treated living things compared to untreated living things, or in diseased living things compared to non-diseased living things. Thus, for example, a t-test can be used to determine whether the mean value of repeated measurements of the level of expression of a particular gene, or protein, is significantly different in a living thing treated with an agent, compared to the same living thing that has not been treated with the agent. Similarly, Analysis of Variance (ANOVA) can be used to compare the mean values of two or more populations (e.g., two or more populations of cultured cells treated with different amounts of a candidate drug) to determine whether the means are significantly different.

The following publications describe examples of art-recognized techniques that can be used to compare the levels of expression of individual genes, or proteins, in treated and untreated living things, or in diseased and non-diseased living things, to identify genes which exhibit significantly different expression levels: Nature Genetics, Vol.32, ps. 461-552 (supplement December 2002); Bioinformatics 18(4):546-54 (April 2002); Dudoit, et al. Technical Report 578, University of California at Berkeley; Tusher et al., Proc. Nat'l. Acad. Sci. U.S.A. 98(9):5116-5121 (April 2001); and Kerr, et al., J. Comput. Biol. 7: 819-837.

Representative examples of other statistical tests that are useful in the practice of the present invention include the chi squared test which can be used, for example, to test for association between two factors (e.g., transcriptional induction, or repression, by a drug molecule and positive or negative correlation with the presence of a disease state). Again by way of example, art-recognized correlation analysis techniques can be used to test whether a correlation exists between two sets of measurements (e.g., between gene expression and disease state). Standard statistical techniques can be found in statistical texts, such as Modern Elementary Statistics, John E. Freund, 7^{th} edition, published by Prentice-Hall; and Practical Statistics for Environmental and Biological Scientists, John Townend, published by John Wiley & Sons, Ltd.

Calculation of an Efficacy Value: An efficacy value can be calculated by measuring the response, to an agent, of each individual gene, or protein, within the efficacy-related population of genes, or efficacy-related population of proteins, to yield a response value for each gene, or protein, within the population, and then performing at least one calculation on all of the response values to yield an efficacy value that numerically represents the expression pattern of the efficacy-related population of genes, or efficacy-related population of proteins, in response to the agent. For example, nucleic acid arrays can be used to measure the response of each individual gene within the efficacy-related gene population, as described supra. Again by way of example, Northern blots may be used to measure the response of each individual gene within the efficacy-related gene population. Measurement of gene expression is usually easier in vitro than in vivo, and an in vitro system is usually better adapted to facilitate high-throughput screening of multiple agents.

An efficacy value can be calculated by any suitable means. For example, a living thing (e.g., a rat heart) is contacted with a reference agent (possessing a known biological activity) in a multiplicity of identical, separate, experiments, and the level of expression of each individual gene, or protein, within an efficacy-related gene or protein population, in response to the reference agent, is measured in each of the multiplicity of experiments. The average expression value for each of the genes, or proteins, is calculated by adding together the expression values from each of the multiplicity of experiments, and dividing the sum by the number of experiments.

The same type of living thing (e.g., a rat heart) is contacted with a candidate agent in a multiplicity of identical, separate, experiments, and the level of expression of each individual gene, or protein, within an efficacy-related gene or protein population, in response to the candidate agent, is measured in each of the multiplicity of experiments. The average expression value for each of the genes, or proteins, is calculated by adding together the expression values from each of the multiplicity of experiments, and dividing the sum by the number of experiments.

The average expression value for each gene in response to the candidate agent is divided by the average expression value for each gene in response to the reference agent to yield a percentage expression value for each gene. The mean of all of the percentage expression values is calculated and is the efficacy value for the candidate agent. Similarly, if protein expression levels are being measured, the average expression value for each protein in response to the candidate agent is divided by the average expression value for each protein in response to the reference agent to yield a percentage expression value for each protein. The mean of all of the percentage expression values is calculated and is the efficacy value for the candidate agent.

By way of further example, the log(ratio)s of the expression levels of all of the genes, or proteins, within an efficacy-related population can be represented by a single scale factor (which is the efficacy value for the agent that caused the gene expression pattern or the protein expression pattern). Exemplary methods for calculating the scale factor S include:

$$( 1 ) . S = {\sum }_{i = 1}^{n} {X}_{i} / {\sum }_{i = 1}^{n} {R}_{i} ;$$

$$n stands for the number of genes and \text{/} or proteins . \\ ( 2 ) . S = ( {\sum }_{i = 1}^{n} {X}_{i} / {R}_{i} ) / n$$

(3). Fit a straight line by: Xᵢ=S*Rᵢ

(4). Least χ² fitting: choose a value of S to minimize the χ²:

$${χ}^{2} = {\sum }_{i = 1}^{n} {( S * {R}_{i} - {X}_{i} )}^{2} / ( {σ}_{Ri}^{2} + {σ}_{Xi}^{2} )$$

(5). Least square fitting: choose a value of S to minimize the Q²:

$${Q}^{2} = {\sum }_{i = 1}^{n} {( S * {R}_{i} - {X}_{i} )}^{2}$$

In the foregoing formulae, Ri, σ_{Ri} stand for the log(Ratio) and error of the log(Ratio) for ith gene, or ith protein, from the template experiment, Xi and σ_{Xi} stand for the log(Ratio) and error of log(Ratio) of the same gene, or protein, expressed in response to a candidate agent. The template experiment is the experiment that yields gene expression data, or protein expression data, in response to an agent having a known biological activity. For example, in the context of using the methods of the invention to identify new agonists of PPARγ, the template experiment is treatment of a living thing with at least one known agonist of PPARγ to yield an efficacy-related gene expression pattern, and/or protein expression pattern, that is characteristic of the known agonist of PPARγ.

Use of a Scale of Efficacy Values: In some embodiments of the methods of this aspect of the invention, an efficacy value of an agent is compared to a scale of efficacy values, typically a continuous scale of efficacy values. The scale of efficacy values can be constructed, for example, by calculating an efficacy value for a reference agent that is known to stimulate a target biological response. This efficacy value forms the upper limit of a continuous scale of efficacy values. The lower limit of the scale can be any value that is less than the efficacy value that forms the upper limit of the scale. For example, the lower limit of the continuous scale can be zero, and the upper limit of the continuous scale can be 1.0. If desired, the scale can be divided into a number of spaced divisions, usually equally spaced divisions, thereby facilitating comparison of an efficacy value of an agent to the scale. For example, a scale that extends from a value of 0 to a value of 1.0 can be divided into the following equally spaced divisions: 0.1, 0.2, 0.3, 0.4, 0.5, 0.6, 0.7, 0.8, 0.9 and 1.0. Optionally, efficacy values can be generated for a multiplicity of reference agents (e.g., 10, 20, 30, 40 or 50 reference agents) that each stimulate the same target, biological, response to different degrees, thereby generating a scale of efficacy values wherein each of the values are actually calculated from expression patterns of an efficacy-related gene population and/or an efficacy-related protein population.

Thus, for example, the upper limit of a continuous scale of efficacy values can be a value of 1.0, which is the efficacy value of a reference agent that is known to stimulate a target biological response. The lower limit of the scale can be arbitrarily set as zero. If the efficacy value of a candidate agent is 0.9, then it can be inferred that the candidate agent is also likely to stimulate the target biological response, because the efficacy value of the candidate agent is close to the efficacy value of the reference agent that is known to stimulate the target biological response.

Toxicity Values and Toxicity-Related Populations of Genes and Proteins: The methods of the invention, for determining whether an agent possesses a defined biological activity, can include the step of comparing a toxicity value of an agent to at least one reference toxicity value to yield a toxicity comparison result, wherein each toxicity value represents at least one expression pattern of the same toxicity-related population of genes or toxicity-related population of proteins. In some embodiments, a toxicity value of the agent is compared to a scale of toxicity values to yield a toxicity comparison result, wherein each toxicity value represents at least one expression pattern of the same toxicity-related population of genes or toxicity-related population of proteins.

A toxicity value is a value that numerically represents the level of expression, in response to an agent, of one of the following: (1) all of the genes within a toxicity-related population of genes; or (2) all of the proteins within a toxicity-related population of proteins. The toxicity-related population of genes, or the toxicity-related population of proteins, yields at least one expression pattern, in response to an agent, that correlates (positively or negatively) with the presence of at least one undesirable biological response caused by the agent in a living thing.

The gene expression pattern of a toxicity-related population of genes, or proteins, induced by an agent, and, therefore, the toxicity value calculated from the induced gene expression pattern, or protein expression pattern, provides an indication of the extent to which an agent induces one or more undesirable effect(s) in a living thing. Thus, the ability of an agent to induce one, or more, undesirable effect(s) in a living thing can be compared to the ability of one or more other agents to induce the same undesirable effect(s) in the same living thing.

It is typically easier, and more readily informative, to compare toxicity values for different agents, than to directly compare the gene expression patterns, or protein expression patterns, induced in a toxicity-related population of genes or proteins by the agents. For example, comparison of toxicity values can be used to determine whether a candidate inhibitor of a target biological response (e.g., a candidate inhibitor of cholesterol synthesis in the mammalian liver) causes the same undesirable biological effects (e.g., destruction of liver cells) as a known inhibitor of the same target biological response. Thus, the toxicity value of the candidate inhibitor of the target biological response is compared to the toxicity value of the known inhibitor of the same target, biological, response to determine whether the two toxicity values are similar. If the toxicity value of the known inhibitor is similar to the toxicity value of the candidate inhibitor, then it is inferred that the candidate inhibitor causes the same, or similar, undesirable biological responses as the known inhibitor.

Again by way of example, in the context of comparing candidate inhibitors of a target biological response to determine which candidate inhibitor is also the weakest inducer of a specific, undesirable, side-effect, the toxicity values of each candidate inhibitor are compared to each other, and it is inferred that the candidate inhibitor that has the numerically smallest toxicity value is the weakest inducer of the undesirable side-effect.

By way of further example, comparison of toxicity values can be used to identify a partial agonist of a specific biological response (e.g., reduction in the amount of glucose in the blood plasma of a diabetic human being). Typically, an agonist of a target biological response elicits more additional biological responses, including undesirable responses, than a partial agonist of the same target biological response. Consequently, partial agonists of a target biological response are usually preferred over agonists of the target biological response for use as therapeutic agents for treating diseases in which the target biological response is malfunctioning. Thus, when screening candidate therapeutic agents that affect the target biological response, it may be desirable to know whether a candidate agent acts more like a known agonist of the target biological response (and so may have more adverse side effects), or whether the candidate agent acts more like a known partial agonist of the target biological response (and so may have fewer adverse side effects). To this end, a population of genes, or proteins, is identified that yields an expression pattern that correlates (positively or negatively) with the induction of one or more undesirable effects in a living thing in response to a known agonist of the target biological response, and that also yields a different expression pattern that correlates (positively or negatively) with the induction of one or more undesirable effects in the same living thing in response to the partial agonist. This is the population of toxicity-related genes or the population of toxicity-related proteins. Typically, the population of toxicity-related genes, or the population of toxicity-related proteins, is the population of toxicity-related genes, or the population of toxicity-related proteins, that yields expression patterns that most clearly distinguish between the agonist and the partial agonist.

A toxicity value is calculated for the agonist, and a toxicity value is calculated for the partial agonist. A toxicity value is also calculated for the candidate agent, and this value is compared to the toxicity value calculated for the agonist, and to the toxicity value calculated for the partial agonist. The result of this comparison reveals whether the gene or protein expression pattern induced by the candidate agent is more like the gene or protein expression pattern induced by the agonist, or is more like the gene or protein expression pattern induced by the partial agonist. In this example, the candidate agent would be selected for further study if its toxicity value is closer to the toxicity value of the known partial agonist than to the toxicity value of the known agonist.

A toxicity-related population of genes or proteins may be identified, for example, by contacting a living thing (e.g., living tissue, living organ or living organism), or population of living things (e.g., population of living cells in culture), with an agent that is known to cause at least one undesirable biological response that is to be measured using the toxicity-related population of genes or proteins. A population of genes or proteins is identified in the living thing that yields at least one expression pattern that correlates (positively or negatively) with the occurrence of the undesirable biological response(s) caused by the agent. This is the toxicity-related population of genes or proteins. The techniques used to measure and analyze gene expression, or protein expression (e.g., gene expression analysis using DNA microarrays, protein expression analysis using protein microarrays) to identify a toxicity-related population of genes or proteins are the same as the techniques that are useful for measuring and analyzing gene expression or protein expression to identify an efficacy-related population of genes or proteins, as described supra.

Example 2 herein describes the identification of toxicity-related populations of genes that are useful for determining whether the undesirable effects induced by a candidate agent in a living thing are more like the undesirable effects induced in the same living thing by a known agonist of PPARγ, or are more like the undesirable effects induced in the same living thing by a known partial agonist of PPARγ.

In some embodiments of the methods of the invention, the toxicity-related population of genes or proteins yields at least one toxicity-related gene expression pattern, in response to an agent, that correlates (positively or negatively) with the presence of at least one undesirable biological response caused by the agent in a living thing, wherein the at least one toxicity-related gene expression pattern, or toxicity-related protein expression pattern, appears before the undesirable biological response. Thus, for example, these embodiments of the methods of the invention are particularly useful for high-throughput screening of numerous drug candidates because it is not necessary to wait for the appearance of the undesirable biological response in order to identify those drug candidates that cause the undesirable biological response.

Calculation of Toxicity Values: A toxicity value is calculated by measuring the response, to an agent, of each individual gene or protein within the toxicity-related gene population, or toxicity-related protein population, to yield a response value for each gene or protein within the population, and then performing at least one calculation on all of the response values to yield a toxicity value that numerically represents the expression pattern of the toxicity-related population of genes, or toxicity-related protein population, in response to the agent. A toxicity value can be calculated by any suitable method, such as the exemplary methods described, supra, for calculating an efficacy value.

Use of a Scale of Toxicity Values: In some embodiments of the methods of this aspect of the invention, a toxicity value of an agent is compared to a scale of toxicity values, typically a continuous scale of toxicity values. The scale of toxicity values can be constructed, and used, with the same techniques useful for constructing and using a scale of efficacy values. For example, a scale of toxicity values can be constructed by calculating a toxicity value for a reference agent that is known to stimulate an undesirable biological response. This toxicity value forms the upper limit of a continuous scale of toxicity values. The lower limit of the scale can be any value that is less than the toxicity value that forms the upper limit of the scale. For example, the lower limit of the continuous scale can be zero, and the upper limit of the continuous scale can be 1.0. Thus, for example, if the toxicity value of a candidate agent is 0.9, then it can be inferred that the candidate agent is likely to stimulate the undesirable biological response, because the toxicity value of the candidate agent is close to the toxicity value of the reference agent that is known to stimulate the undesirable biological response.

Classifier Values: The methods of this aspect of the invention can include the step of comparing a classifier value of an agent to at least one reference classifier value to yield a classifier comparison result, wherein each classifier value represents at least one expression pattern of the same classifier population of genes, or classifier population of proteins. In some embodiments, a classifier value of the agent is compared to a scale of classifier values to yield a classifier comparison result, wherein each classifier value represents at least one expression pattern of the same classifier population of genes, or classifier population of proteins.

A classifier value numerically represents the level of expression, in response to an agent, of one of the following: (1) all of the genes within a classifier population of genes; or (2) all of the proteins within a classifier population of proteins. A classifier population of genes or proteins yields different gene expression patterns, or protein expression patterns, and different calculated classifier values, in response to different reference agents that have different biological activities (e.g., an agonist and a partial agonist of the same target biological response). The gene expression pattern, or protein expression pattern, induced by an agent in the classifier population of genes or proteins correlates (positively or negatively) with the occurrence of the biological activity of the agent. Thus, the biological activities of different agents can be grouped into one, or more, classes based on the gene expression pattern, or protein expression pattern, induced by an agent in one, or more, classifier population(s) of genes or proteins. It is typically easier, and more readily informative, to compare classifier values for different agents, than to compare the gene expression patterns from which the classifier values are calculated.

Thus, for example, the classifier value of a candidate agent (e.g., a candidate therapeutic drug molecule) can be compared to the classifier value of a first reference agent that possesses a known biological activity, and to the classifier value of a second reference agent, that possesses a known biological activity that is different from the biological activity of the first reference agent. The comparison reveals whether the gene expression pattern, or protein expression pattern, induced by the candidate agent (and, by implication, the biological activity of the candidate agent) is more like the gene expression pattern, or protein expression pattern, induced by the first reference agent, or is more like the gene expression pattern, or protein expression pattern, induced by the second reference agent. The biological activity of the candidate agent can thereby be classified as being more like the first reference agent, or as being more like the second reference agent.

By way of specific example, the first reference agent may be an agonist of a target biological response in a living thing, and the second reference agent may be a partial agonist of the same target biological response in the same living thing. The agonist stimulates the target biological response in the living thing, but also stimulates other biological responses which may be toxic, or otherwise undesirable, to the living thing. The partial agonist stimulates the same target biological response as the agonist, but stimulates fewer, potentially undesirable, biological responses compared to the agonist. Thus, an agonist is likely to have more undesirable side effects than a partial agonist.

To determine whether a candidate agent has a biological activity that is more like the biological activity of an agonist of a specific biological response, or is more like the biological activity of a partial agonist of the same biological response, a living thing is contacted with the candidate agent, and the expression pattern of a classifier population of genes, or the expression pattern of a classifier population of proteins, in the living thing is measured. The classifier population of genes, or classifier population of proteins, yields a different expression pattern, and, hence, a different calculated classifier value, in response to the agonist than in response to the partial agonist. A classifier value is calculated for the agonist, and a classifier value is calculated for the partial agonist. A classifier value is also calculated for the candidate agent, and this value is compared to the classifier value calculated for the agonist, and to the classifier value calculated for the partial agonist. The result of this comparison reveals whether the gene expression pattern, or protein expression pattern, induced by the candidate agent is more like the gene expression pattern, or protein expression pattern, induced by the agonist, or is more like the gene expression pattern, or protein expression pattern, induced by the partial agonist.

A classifier population of genes, or classifier population of proteins, can be identified, for example, by contacting a living thing (e.g., living tissue, living organ or living organism), or population of living things (e.g., population of living cells in culture), with an agent that is known to cause a target biological response. A population of genes, or a population of proteins, is identified in the living thing that yields at least one expression pattern that correlates (positively or negatively) with the occurrence of the target biological response caused by the agent. The foregoing procedure is repeated with a second reference agent, possessing a different biological activity than the first reference agent, to yield a gene expression pattern, or a protein expression pattern, that is characteristic of the second reference agent. The gene expression pattern, or protein expression pattern, of the first reference agent, and the gene expression pattern, or protein expression pattern, of the second reference agent, are compared to identify the population of genes, or proteins (within the total population of genes, or proteins, whose expression is affected by either the first or second reference agents) that produces an expression pattern that most clearly distinguishes between the first reference agent and the second reference agent. This population of genes, or proteins, is the classifier population. It is understood that the same general method can be used to identify a classifier population of genes, or a classifier population of proteins, that distinguishes between two or more reference agents.

Classifier populations of genes can be identified, for example, in the following manner. Living cells are contacted, in vivo or in vitro, with an amount of a first reference agent that maximally induces (or maximally inhibits) a target biological response. Messenger RNA is extracted from the contacted cells and used as a template to synthesize cDNA which is then labeled (e.g., with a fluorescent dye). The labeled cDNA is used to probe a DNA array that includes hundreds, or thousands, of identified nucleic acid molecules (e.g., cDNA molecules) that correspond to genes that are expressed in the type of cells that were contacted with the first reference agent. The labeled cDNA molecules that hybridize to the nucleic acid molecules immobilized on the DNA array are identified, and the level of expression of each hybridizing cDNA is measured and compared to the level of expression of the same mRNA molecules in a control sample from living cells that were not contacted with the first reference agent, to yield a gene expression pattern that is induced by the first reference agent.

The foregoing procedure is repeated with a second reference agent, possessing a different biological activity compared to the first reference agent, to yield a gene expression pattern that is characteristic of the second reference agent. For example, the first reference agent may be an agonist of a biological response, and the second reference agent may be a partial agonist of the same biological response. The gene expression pattern of the first reference agent, and the gene expression pattern of the second reference agent, are compared to identify the population of genes (within the total population of genes whose expression is affected by either the first or second reference agents) that produces an expression pattern that most clearly distinguishes between the first reference agent and the second reference agent. This population of genes is the classifier population. In the context of the present example, the classifier population permits classification of a candidate agent as being more similar to the first reference agent than to the second reference agent, or as being more similar to the second reference agent than to the first reference agent. Example 3 herein describes the identification of a classifier population of genes that is useful for classifying candidate agents as being more like an agonist of PPARγ, or as being more like a partial agonist of PPARγ.

Classifier populations of proteins can be identified, for example, using the same foregoing approach for identifying classifier populations of genes, except that techniques for measuring the amount of individual proteins (e.g., two dimensional gel electrophoresis) are used instead of techniques for measuring the amount of individual genes.

Calculating a Classifier Value: A classifier value is calculated by measuring the response, to an agent, of each individual gene, or protein, within the classifier gene population, or within the classifier protein population, to yield a response value for each gene within the population, or each protein within the population, and then performing a calculation on all of the response values to yield a classifier value that numerically represents the expression pattern of the classifier population of genes, or proteins, in response to the agent. A classifier value can be calculated by any suitable method, such as the exemplary methods described, supra, for calculating an efficacy value.

Use of a Scale of Classifier Values: In some embodiments of the methods of this aspect of the invention, a classifier value of an agent is compared to a scale of classifier values, typically a continuous scale of classifier values. The scale of classifier values can be constructed, and used, with the same techniques useful for constructing and using a scale of efficacy values or toxicity values. For example, a scale of classifier values can be constructed by generating classifier values for two reference agents. For example, the classifier value for a partial agonist of a biological response may be 0.1, and the classifier value for an agonist of the same biological response may be 1.0. Thus, the scale of classifier values extends from 0.1 (the classifier value that is most characteristic of a partial agonist of the biological response), to 1.0 (the classifier value that is most characteristic of an agonist of the biological response). Thus, for example, the classifier value of a candidate agent may be 0.6, which is closer to the classifier value of the agonist (1.0), than to the classifier value of the partial agonist (0.1), suggesting that the candidate agent is more likely to be an agonist of the target biological response than a partial agonist of the target biological response.

Practicing the methods of the invention in vitro: In some embodiments of the methods of the invention, the expression pattern of one, or more, of the classifier population of genes (or classifier population of proteins), the toxicity-related population of genes (or toxicity-related population of proteins), and the efficacy-related population of genes (or efficacy-related population of proteins) is/are measured in the same population of living cells cultured in vitro. The use of a population of living cells, cultured in vitro, to measure gene expression patterns, or protein expression patterns, facilitates rapid, high throughput, screening of numerous agents. Representative examples of living cells that can be cultured in vitro and used in the practice of the present invention to measure the expression pattern of one, or more, of the classifier population of genes (or classifier population of proteins), the toxicity-related population of genes (or toxicity-related population of proteins), and the efficacy-related population of genes (or efficacy-related population of proteins), are 3T3L1 adipocyte cells (available from the American Type Culture Collection, Manassas, Va., as cell line CL-173), hepatocyte cells, myocardiocyte cells, human primary hepatocytes and HEPG2 cells (available from the American Type Culture Collection, Manassas, Va., as cell line HB-8065).

Typically, but not necessarily, cultured cells are chosen that correspond to the cells that are affected, in vivo, by the agent(s) whose biological activity will be assessed using the cultured cells. For example, cultured liver cells may be used in the practice of the methods of the invention to screen candidate chemical agents that affect an aspect of liver metabolism (e.g., cholesterol synthesis). Similarly, cultured myocardiocyte cells may be used in the practice of the methods of the invention to screen candidate chemical agents that affect an aspect of heart cell metabolism, or cardiac function. Again by way of example, cultured human myoblasts may be used to identify agents that possess the undesirable property of causing cardiac myopathy.

In some embodiments of the methods of the invention, the expression pattern of at least one member of the group consisting of the classifier population of genes (or classifier population of proteins), the toxicity-related population of genes (or toxicity-related population of proteins), and the efficacy-related population of genes (or efficacy-related population of proteins) is measured in vivo, and the expression pattern of at least one of the foregoing populations of genes or proteins is measured in vitro. For example, chemical agents that affect an aspect of cardiac function (e.g., reduce heart size in a human subject suffering from cardiomyopathy) may be identified by measuring the expression of an efficacy-related gene population in heart tissue of experimental animals treated with candidate agents. Undesirable adverse effects of the candidate agents can be identified by measuring the expression of a toxicity-related gene population in a cardiomyocyte cell population cultured in vitro.

In some embodiments, the expression pattern of a toxicity-related population of genes (or toxicity-related population of proteins), and/or the expression pattern of an efficacy-related population of genes (or efficacy-related population of proteins) is/are measured, in vitro, using cultured cells that are different from the type(s) of cells that are predominantly (or exclusively) affected, in vivo, by the agent(s) whose biological activity will be assessed using the cultured cells. In these embodiments, the living cells that are used to measure the expression pattern of the toxicity-related population of genes (or toxicity-related population of proteins), and/or the expression pattern of the efficacy-related population of genes (or efficacy-related population of proteins), are typically easier to culture and assay than the cells that suffer the undesirable biological effect(s), or exhibit the desired biological effect(s), in vivo.

For example, one type of undesirable effect caused by some therapeutic molecules (e.g., rosiglitazone) administered to mammalian subjects is enlargement of the heart, which may also be accompanied by an increase in blood plasma volume. One way to measure these types of undesirable effects is to measure the gene expression pattern of a toxicity-related population of genes in heart tissue of experimental animals (e.g., rats) treated with agents that cause these effects. In some embodiments of the methods of the present invention, however, a more convenient way to measure these changes is to identify cells or tissue that are culturable in vitro, and that exhibit changes in gene expression that correlate with, and preferably precede, the changes in heart size and/or plasma volume observed in vivo. An example of culturable mammalian cells that meet the foregoing criteria with respect to changes in gene expression are mouse 3T3L1 adipocyte cells.

As described in Example 2, in one option for using 3T3L1 adipocyte mouse cells in the practice of the invention, one, or more, of a classifier population of genes, a toxicity-related population of genes, and an efficacy-related population of genes is/are identified in rat epididymal white adipose tissue (EWAT), in vivo, in accordance with the teachings of the present patent application. Thereafter, the classifier population of genes, and/or the toxicity-related population of genes, and/or the efficacy-related population of genes is/are mapped onto 3T3L1 mouse adipocytes.

Use of the classifier comparison result, and/or toxicity comparison result, and/or efficacy comparison result to determine whether an agent possesses a defined biological activity: In the practice of the methods of the present invention, one or more of the classifier comparison result, the toxicity comparison result, and/or the efficacy comparison result is/are used to determine whether an agent possesses a defined biological activity. For example, any one of the classifier comparison result, the toxicity comparison result, or the efficacy comparison result may be used alone to determine whether an agent possesses a defined biological activity. More typically, one of the following combinations of comparison results is used to determine whether an agent possesses a defined biological activity: efficacy comparison result and toxicity comparison result; efficacy comparison result and classifier comparison result; classifier comparison result and toxicity comparison result; toxicity comparison result and efficacy comparison result and classifier comparison result.

The choice of which comparison result, or combination of comparison results, to use to determine whether an agent possesses a defined biological activity, and the weight to give each comparison result when a combination of comparison results is used, mainly depends on the type and magnitude of the defined biological activity that candidate agents desirably possess. The precise weight to give to a comparison result is a decision that is made in the context of a particular experiment, and is a matter of judgment. For example, an investigator might identify a population of chemical compounds that are potent stimulants of a target biological process, and are therefore candidate therapeutic agents for treating diseased subjects in which the target biological process is inactive, or active at a low level, thereby causing disease. The investigator may want to identify those compounds within the population that cause the least number of undesirable side effects. Thus, for example, the investigator may use only the toxicity comparison result to select candidate therapeutic agents (that cause the least number of undesirable side effects) from among the population of chemical compounds that stimulate the target biological response. If the investigator uses one or more comparison results in addition to the toxicity comparison result, such as the combination of the toxicity comparison result and the efficacy comparison result, the investigator may give most weight to the toxicity comparison result since, in this example, all of the compounds are about equally effective stimulants of the target biological process, and the investigator is most interested in identifying those compounds that cause fewest adverse side-effects.

Again by way of example, an investigator might want to identify a chemical compound that is a potent stimulant of a target biological response, but which does not induce a defined, undesirable, side effect. Thus, the investigator may use the combination of an efficacy comparison result and a toxicity comparison result to determine whether an agent is a potent stimulant of the target biological response, but does not induce the undesirable side effect. Since, in this example, the investigator considers the ability of a compound to stimulate the target biological response to be about equally important as the inability of the compound to induce the undesirable side effect, the investigator may give equal weight, or approximately equal weight, to the efficacy comparison result and to the toxicity comparison result.

The use of other comparison results, in addition to an efficacy comparison result, and/or a toxicity comparison result, and/or a classifier comparison result, is also within the scope of the invention. Thus, using the techniques described herein, a comparison result can be obtained for any measurable biological response. For example, agonists and partial agonists of PPARγ receptors may also stimulate a related class of molecules called PPARα receptors. Thus, using the techniques described herein, a population of genes, or proteins, can be identified that yield an expression pattern that correlates (positively or negatively) with the stimulation of PPARα receptors by an agent. This population of genes, or proteins, can be used to screen candidate PPARγ agonists, or partial agonists, to identify those candidate agents that possess the undesirable property of stimulating PPARα receptors.

In another aspect, the present invention provides populations of nucleic acid molecules that are useful in the practice of the methods of the present invention as probes for measuring the level of expression of members of a classifier population of genes, or an efficacy-related population of genes, or a toxicity-related population of genes, wherein the classifier population of genes, the efficacy-related population of genes, and the toxicity-related population of genes are each useful for identifying agonists, or partial agonists, of PPARγ.

In a further aspect, the present invention provides populations of oligonucleotide probes and populations of genes. The populations of genes include classifier populations of genes, efficacy-related populations of genes, and toxicity-related populations of genes, and are useful, for example, for determining whether an agent possesses a defined biological activity in accordance with the teachings of the present patent application. The populations of oligonucleotide probes are useful, for example, for measuring the expression patterns of classifier populations of genes, efficacy-related populations of genes, or toxicity-related populations of genes of the present invention.

For example, as more fully described in Example 1 herein, Table 1, entitled “PPARg_Mouse_Efficacy_Probe_{—}52 (Species: db/db Mouse)”, sets forth an efficacy-related population of mouse genes (SEQ ID NOs: 1-50). The population of 52 oligonucleotide probes identified in Table 1 (SEQ ID NOs: 51-102), and the population of 22 oligonucleotide probes (SEQ ID NOs: 52, 53, 58, 59, 65, 66, 68, 69, 71, 73, 75, 76, 78, 82, 86, 88-90, 93, 94, 96, 101) identified in Table 2, entitled “PPARg_{—}3T3L1_Efficacy_Probe_{—}22 (Species: Mouse Cell Line)”, are useful in the practice of the methods of the invention to measure the expression pattern of some or all of the efficacy-related population of genes (SEQ ID NOs: 1-50) described in Table 1.

Again by way of example, as more fully described in Example 2 herein, Table 4 sets forth a rat toxicity-related population of genes (SEQ ID NOs: 103-152), and a population of oligonucleotide probes (SEQ ID NOs: 153-207) that are useful in the practice of the present invention to measure the expression pattern of the toxicity-related population of genes (SEQ ID NOs: 103-152). Again by way of example, Table 5 sets forth a toxicity-related population of 5 mouse genes (SEQ ID NOs: 208-212) that are useful as early reporters of heart toxicity. Table 5 sets forth a population of oligonucleotide probes (SEQ ID NOs: 213-218) that are useful for measuring the expression pattern of the toxicity-related population of 5 genes (SEQ ID NOs: 208-212).

Again by way of example, Table 6 sets forth a rat toxicity-related population of genes (SEQ ID NOs: 219-550, 104, 105, 112, 119, 126, 127, 133, 136, 149, 150 and 151), and a population of oligonucleotide probes (SEQ ID NOs: 551-894, 155, 157, 164, 171, 178, 179, 185, 188, 204, 205, and 206) that are useful in the practice of the present invention to measure the expression pattern of the toxicity-related populations of genes (SEQ ID NOs: 219-550, 104, 105, 112, 119, 126, 127, 133, 136, 149, 150 and 151).

Table 7 sets forth a mouse cell line toxicity-related population of genes (SEQ ID NOs: 895-949, 42 and 45), and a population of oligonucleotide probes (SEQ ID NOs: 950-1019, 863, 93, 94, and 97) that are useful in the practice of the present invention to measure the expression pattern of the toxicity-related populations of genes (SEQ ID NOs: 895-949, 42 and 45).

Table 8 sets forth a mouse tissue toxicity-related population of genes (SEQ ID NOs: 1020-1035, 896, 900, 902, 903, 905, 906, 13, 908, 912, 917-920, 925, 926, 929, 932, 934, 936-938, 42, 939, 942, 45, 943-946 and 949), and a population of oligonucleotide probes (SEQ ID NOs: 1036-1057, 951, 955, 957, 863, 959, 960, 63, 962, 966, 971-974, 980, 981, 984, 987, 989, 991-996, 93, 998, 94, 999-1001, 1004, 97, 1005-1014, and 1017-1019) that are useful in the practice of the present invention to measure the expression pattern of the toxicity-related populations of genes (SEQ ID NOs: 1020-1035, 896, 900, 902, 903, 905, 906, 13, 908, 912, 917-920, 925, 926, 929, 932, 936-938, 42, 939, 942, 45, 943-946 and 949).

Table 9 sets forth a rat tissue toxicity-related population of genes (SEQ ID NOs: 1058-1238, 222, 224, 106, 226, 235, 237, 239, 246, 253, 258, 261, 270, 273, 274, 278, 111, 286, 302-304, 307, 308, 316-318, 322, 327, 119, 342, 358, 361, 367-368, 373, 381, 388, 401, 406, 409-410, 416-418, 423, 427-428, 430-432, 434, 439, 441, 447, 450, 455, 461, 464-465, 136, 137, 139, 474, 475, 482, 485, 488, 491, 492, 496, 500, 504, 524, 530, 534, 536, 541, 542, and 547), and a population of oligonucleotide probes (SEQ ID NOs: 1239-1428, 558, 561, 158, 565, 574, 576, 578, 585, 592, 597, 600, 609, 612, 613, 617, 163, 625, 641-643, 646, 647, 655-657, 661, 666, 171, 681, 697, 700, 706, 707, 712, 720, 727, 740, 745, 748, 749, 755-757, 762, 766-767, 769-771, 773, 778, 780, 786, 789, 794, 800, 803-804, 188-189, 191, 813-814, 822-823, 556, 828, 831-832, 836, 840, 844, 864, 871, 876, 878, 883, 884, 889-891) that are useful in the practice of the present invention to measure the expression pattern of the toxicity-related populations of genes (SEQ ID NOs: 1058-1238, 222, 224, 106, 226, 235, 237, 239, 246, 253, 258, 261, 270, 273, 274, 278, 111, 286, 302-304, 307, 308, 316-318, 322, 327, 119, 342, 358, 361, 367-368, 373, 381, 388, 401, 406, 409-410, 416-418, 423, 427-428, 430-432, 434, 439, 441, 447, 450, 455, 461, 464-465, 136, 137, 139, 474, 475, 482, 485, 488, 491, 492, 496, 500, 504, 524, 530, 534, 536, 541, 542, and 547).

Table 10 sets forth a mouse cell line toxicity-related population of genes (SEQ ID NOs: 1429-1448, 897, 901, 902, 919, 921, 922, 926, 928, 929, 931, 935, 939, 942, 943, and 946), and a population of oligonucleotide probes (SEQ ID NOs: 1449-1471, 952, 956, 957, 973, 975-976, 981, 983, 984, 986, 990, 999-1001, 1004-1007, and 1012-1014) that are useful in the practice of the present invention to measure the expression pattern of the toxicity-related populations of genes (SEQ ID NOs: 1429-1448, 897, 901, 902, 919, 921, 922, 926, 928, 929, 931, 935, 939, 942, 943, and 946).

Table 12 sets forth a mouse cell line classifier population of genes (SEQ ID NOs: 1472-1730, 2, 896, 1429, 902, 1431, 1434, 15, 18, 19, 22, 25, 1436, 913, 1437, 916, 917, 920, 1441, 32, 923, 927, 39, 934, 935, 210, 939, 44, 1445, 943, 212, 946, 949), and a population of oligonucleotide probes (SEQ ID NOs: 1731-1996, 52, 951, 1450, 957, 1452, 1455, 65, 68, 69, 72, 75, 1457, 967, 1458, 970, 971, 974, 1462, 82, 977-978, 982, 90, 989, 990, 215, 1001, 999, 1000, 96, 1468, 1005-1006, 1970, 218, 1014, 1018, and 1019) that are useful in the practice of the present invention to measure the expression pattern of the classifier populations of genes (SEQ ID NOs: 1472-1730, 2, 896, 1429, 902, 1431, 1434, 15, 18, 19, 22, 25, 1436, 913, 1437, 916, 917, 920, 1441, 32, 923, 927, 39, 934, 935, 210, 939, 44, 1445, 943, 212, 946, 949).

Table 14 sets forth a mouse cell line population of genes (SEQ ID NOs: 1997-2795, 1473, 1475, 3, 1481, 1429, 1488, 1489, 1021, 1500, 902, 1515, 10, 1521, 13, 1538, 908, 1549, 1025, 1550, 1558, 1559, 1561, 1565, 21, 22, 1574, 912, 1614, 916-919, 1620, 1030, 1031, 922, 1639, 1645, 30, 1651, 35, 1673, 1674, 1682, 1033, 934, 1694, 936, 1034, 937, 210, 42, 939, 1444, 1698, 940, 209, 1703, 943, 1035, 945, 1710, 946, 1711, 1712, 1714, 948, 949, 142, 1728, and 49) that yield an expression pattern that correlates with the stimulation of PPARα receptors by an agent, and a population of oligonucleotide probes (SEQ ID NO. 2796-3683, 1732, 1734, 53, 1740, 1449, 1450, 1747, 1748, 1037, 1759, 957, 1774, 60, 1780, 63, 1797, 962, 1808, 1041, 1809, 1817, 1818, 1820, 1824, 71, 72, 1833, 966, 1873, 970-973, 1879, 1046, 1047, 976, 1898, 1904, 80, 1910, 86, 1932, 1933, 1941, 1049, 989, 1953, 991-993, 1050, 1051, 994, 215, 216, 93, 94, 998-1001, 1465-1467, 1957, 1002, 214, 1962, 1005-1007, 1056, 1057, 1009-1014, 1974, 1975, 1977, 1979, 1016-1019, 1994, 101) that are useful in the practice of the present invention to measure the expression pattern of the foregoing populations of genes (SEQ ID NOs: 1997-2795, 1473, 1475, 3, 1481, 1429, 1488, 1489, 1021, 1500, 902, 1515, 10, 1521, 13, 1538, 908, 1549, 1025, 1550, 1558, 1559, 1561, 1565, 21, 22, 1574, 912, 1614, 916-919, 1620, 1030, 1031, 922, 1639, 1645, 30, 1651, 35, 1673, 1674, 1682, 1033, 934, 1694, 936, 1034, 937, 210, 42, 939, 1444, 1698, 940, 209, 1703, 943, 1035, 945, 1710, 946, 1711, 1712, 1714, 948, 949, 142, 1728, and 49).

Methods for identifying an efficacy-related population of genes or proteins: In another aspect, the present invention provides methods for identifying an efficacy-related population of genes or proteins which are useful, for example, in the practice of the methods of the present invention for determining whether an agent possesses a defined biological activity. The methods of this aspect of the invention include the steps of (a) contacting a living thing with an agent that is known to elicit a desired biological response; and (b) identifying an efficacy-related population of genes or proteins in the living thing that yields an expression pattern that correlates with the occurrence of the desired biological response caused by the agent.

In some embodiments, the expression pattern of the efficacy-related population of genes or proteins appears in the living thing before the occurrence of the desired biological response caused by the agent. In some embodiments, the desired biological response does not occur in the living thing. For example, the living thing may be rat epididymal white adipose tissue which includes an efficacy-related population of genes, or proteins, that yields an expression pattern that correlates with the occurrence of a reduction in the concentration of glucose in rat's blood in response to a chemical agent administered to the rat. The expression pattern of the efficacy-related population of genes or proteins appears, however, before the reduction in blood glucose concentration.

Some embodiments of the methods of this aspect of the invention include the following steps: (a) measuring the level of expression of each member of a multiplicity of genes or proteins in the living thing, contacted with the agent, to yield a multiplicity of expression values; (b) measuring the level of expression of each member of the same multiplicity of genes or proteins in a reference living thing, that is not contacted with the agent, to yield a multiplicity of reference expression values; and (c) comparing the multiplicity of expression values with the multiplicity of reference expression values to identify an efficacy-related population of genes or proteins, wherein each individual gene or protein has an expression value in response to the agent that is significantly different from the corresponding reference expression value.

The reference living thing can be the living thing that is contacted with the agent before it is contacted with the agent. For example, a sample of cells or tissue may be removed from the living thing before it is contacted with the agent; thereafter, the living thing is contacted with the agent and a further sample of cells or tissue is removed from the living thing, and gene expression is analyzed and compared between the two samples. The reference living thing can also be the same type of cells, tissue, organ or organism as the living thing contacted with the agent, except that the reference living thing is not contacted with the agent. For example, the living thing can be a db/db mouse to which is administered a dosage of rosiglitazone, and the reference living thing can be a different db/db mouse which is not administered a dosage of rosiglitazone. It is understood that typically a population of living things, and reference living things, are used in the practice of this aspect of the invention to provide a sufficiently large number of data for statistical analysis.

Some agents elicit more than one biological response in a living thing (e.g., more than one desirable biological response, or more than one undesirable biological response, or at least one desirable biological response and at least one undesirable biological response). Elicitation of a biological response may require the action of a target molecule (e.g., protein receptor). Typically, the target molecule is a component of a biochemical signal transduction pathway that is affected by the agent, and that conveys one, or more, biochemical signals (typically in the form of organic molecules, such as lipids) that elicit the biological response. For example, an agent may directly, physically, interact with a target molecule (e.g., a protein receptor molecule located in a cell membrane) to elicit a desired biological response. Again by way of example, an agent may directly, physically, interact with a molecule, and this interaction may trigger the release of one or more signalling molecules that move within and/or between cells. One of these signalling molecules interacts with a target molecule (e.g., a protein receptor molecule) to elicit a desired biological response.

A first target molecule may be required to elicit a first biological response when a living thing is contacted with an agent, and a second target molecule, that is different from the first target molecule, may be required to elicit a second biological response when the same living thing is contacted with the same agent. In one aspect, the present invention provides methods that can be used to identify an efficacy-related population of genes or proteins that yields an expression pattern that correlates with the occurrence of only the first or the second desired biological response caused by the direct, or indirect, interaction of the agent with one of two types of target molecules. These methods include the steps of (a) contacting the living thing with an agent that is known to elicit at least two different desired biological responses in the living thing, wherein elicitation of a first desired biological response by the agent is mediated by a first target molecule, and elicitation of a second desired biological response by the agent is mediated by a second target molecule that is different from the first target molecule; (b) identifying an efficacy-related population of genes or proteins that yields an expression pattern that correlates with the occurrence of the first and second desired biological responses in response to the agent; (c) contacting a modified living thing with the agent, wherein the modified living thing is a member of the same species as the living thing and does not include any functional first target molecules; (d) identifying an efficacy-related population of genes or proteins that yields an expression pattern that correlates with the occurrence of the second desired biological response in the modified living thing in response to the agent; and (e) comparing the efficacy-related population of genes or proteins identified in step (b) with the efficacy-related population of genes or proteins identified in step (d) to identify an efficacy-related population of genes or proteins that yields an expression pattern that correlates with the occurrence of the first desired biological response caused by the agent.

It is understood that steps (a) through (d) can be in any temporal sequence (e.g., steps (c) and (d) can be practised, to identify an efficacy-related population of genes or proteins that yields an expression pattern that correlates with the occurrence of the second target biological response, before steps (a) and (b) are practised to identify a population of genes or proteins that yields an expression pattern that correlates with the occurrence of the first and second target biological responses in response to the agent. The modified living thing can be, for example, a so-called “knockout” organism (or cells or tissues derived from a “knockout” organism) which has been genetically modified, for example by the process of targeted homologous recombination, to inactivate all genes encoding a target molecule.

Methods for identifying a toxicity-related population of genes or proteins: In another aspect, the present invention provides methods for identifying a toxicity-related population of genes or proteins which are useful, for example, in the practice of the methods of the present invention for determining whether an agent possesses a defined biological activity. The methods of this aspect of the invention include the steps of (a) contacting a living thing with an agent that is known to elicit an undesirable biological response; and (b) identifying a toxicity-related population of genes or proteins that yields an expression pattern that correlates with the occurrence of the undesirable biological response caused by the agent.

In some embodiments, the expression pattern of the toxicity-related population of genes or proteins appears in the living thing before the occurrence of the undesirable biological response caused by the agent. In some embodiments, the undesirable biological response does not occur in the living thing.

Some embodiments of the methods of this aspect of the invention include the following steps: (a) measuring the level of expression of each member of a multiplicity of genes or proteins in the living thing, contacted with the agent, to yield a multiplicity of expression values; (b) measuring the level of expression of each member of the same multiplicity of genes or proteins in a reference living thing, that is not contacted with the agent, to yield a multiplicity of reference expression values; and (c) comparing the multiplicity of expression values with the multiplicity of reference expression values to identify a toxicity-related population of genes or proteins, wherein each individual gene or protein has an expression value in response to the agent that is significantly different from the corresponding reference expression value.

As described, supra, in connection with the methods of the invention for identifying an efficacy-related population of genes or proteins, the reference living thing can be the living thing that is contacted with the agent before it is contacted with the agent. The reference living thing can also be the same type of cells, tissue, organ or organism as the living thing contacted with the agent, except that the reference living thing is not contacted with the agent. It is understood that typically a population of living things, and reference living things, are used in the practice of this aspect of the invention to provide a sufficiently large number of data for statistical analysis.

Some embodiments of the methods of this aspect of the invention permit a user to distinguish between the expression pattern of an efficacy-related population of genes or proteins, and the expression pattern of a toxicity-related population of genes or proteins, wherein both expression patterns are caused by the same agent, and elicitation of the two expression patterns is mediated by two different target molecules. These embodiments include the steps of (a) contacting a living thing with an agent that is known to elicit a desirable biological response and an undesirable biological response in the living thing, wherein elicitation of the desirable biological response is mediated by a first target molecule, and elicitation of the undesirable biological response is mediated by a second target molecule that is different from the first target molecule; (b) identifying a population of genes or proteins that yields an expression pattern that correlates with the occurrence of the desirable and undesirable biological responses caused by the agent; (c) contacting a modified living thing with the agent, wherein the modified living thing is a member of the same species as the living thing and does not include any functional second target molecules; (d) identifying an efficacy-related population of genes or proteins that yields an expression pattern that correlates with the occurrence of the desirable biological response caused by the agent; and (e) comparing the population of genes or proteins identified in step (b) with the efficacy-related population of genes or proteins identified in step (d) to identify a toxicity-related population of genes or proteins that yields an expression pattern that correlates with the occurrence of the undesirable biological response caused by the agent. By way of specific example, the first target molecule can be a PPARγ receptor and the second target molecule can be a PPARα receptor.

In the context of the methods of this aspect of the invention, the terms “elicitation of the desirable biological response is mediated by a first target molecule” and “elicitation of the undesirable biological response is mediated by a second target molecule” mean that the target molecule is a component of the biochemical signal transduction pathway that is affected by the agent, and that conveys one, or more, biochemical signals (typically in the form of organic molecules, such as lipids) that elicit the desirable, or undesirable, biological response.

It is understood that steps (a) through (d) can be in any temporal sequence. The modified living thing can be, for example, a so-called “knockout” organism (or cells or tissues derived from a “knockout” organism) which has been genetically modified, by the process of targeted homologous recombination, to inactivate all genes encoding a target molecule.

Methods for identifying a classifier population of genes or proteins: In another aspect, the present invention provides methods for identifying a classifier population of genes or proteins, which are useful, for example, in the practice of the methods of the present invention for determining whether an agent possesses a defined biological activity. The methods of this aspect of the invention include the steps of (a) contacting a living thing with a first reference agent that is known to cause a first biological response;
(b) identifying a first population of genes or proteins that yields an expression pattern that correlates with the occurrence of the first biological response caused by the first reference agent; (c) contacting a living thing with a second reference agent that is known to cause a second biological response, wherein the living thing is the same living thing that is contacted with the first reference agent, or is a different living thing that is a member of the same species as the living thing that is contacted with the first reference agent; (d) identifying a second population of genes or proteins that yields an expression pattern that correlates with the occurrence of the second biological response caused by the second reference agent; and (e) comparing the first population of genes or proteins to the second population of genes or proteins and thereby identifying a classifier population of genes or proteins that produces an expression pattern that most clearly distinguishes between the first reference agent and the second reference agent. It is understood that the combination of step (a) and step (b) can be performed before, during or after the combination of step (c) and step (d).

The following examples merely illustrate the best mode now contemplated for practicing the invention, but should not be construed to limit the invention.

### EXAMPLE 1

This Example describes the identification of two efficacy-related populations of genes that are both useful in the practice of the methods of the invention for identifying agonists and partial agonists of PPARγ. One efficacy-related population of 50 genes was identified in mouse EWAT tissue. The nucleotide sequences of these 50 genes are set forth in the portion of this patent application entitled SEQUENCE LISTING and are identified in Table 1, (SEQ ID NOs: 1-50). The nucleotide sequences of the 52 oligonucleotide probes used to measure the expression levels of these 50 genes (SEQ ID NOs: 1-50) are set forth in the SEQUENCE LISTING and identified in Table 1, (SEQ ID NOs: 51-102). The other efficacy-related population of genes includes 21 genes that were identified in cultured 3T3L1 mouse adipocyte cells (passages 3-9). These 21 genes, whose nucleotide sequences are set forth in the SEQUENCE LISTING (SEQ ID NOs: 2, 3, 8, 9, 15, 16, 18, 19, 21, 23, 25, 26, 28, 32, 35, 37-39, 42, 44, 49), are a subset of the foregoing 50 genes. The oligonucleotide probes used to measure the expression levels of these 21 genes (SEQ ID NOs: 2, 3, 8, 9, 15, 16, 18, 19, 21, 23, 25, 26, 28, 32, 35, 37-39, 42, 44, 49) are identified in Table 2, (SEQ ID NOs: 52, 53, 58, 59, 65, 66, 68, 69, 71, 73, 75, 76, 78, 82, 86, 88-90, 93, 94, 96, 101).

TABLE 1
PPARγ_Mouse_Efficacy_Probe_52 (Species: db/db Mouse)

| Accession | | Gene SEQ | Probe SEQ |
|---|---|---|---|
| number | Gene Name | ID NO | ID NO |
| AK010455 | 2410008K03Rik | 1 | 51 |
| AW909114 | MGC28611 | 2 | 52 |
| NM_008543 | Madh7 | 3 | 53 |
| AF282730 | Timp4 | 4 | 54 |
| M12347 | Acta1 | 5 | 55 |
| NM_007377 | Aatk | 6 | 56 |
| AK002237 | Gadd45g | 7 | 57 |
| NM_030701 | Pumag-pending | 8 | 58 |
| AK012169 | Slitl2 | 9 | 59 |
| AV279434 | 4930458D05Rik | 10 | 60 |
| NM_022020 | Rbp7 | 11 | 61 |
| NM_019738 | Nupr1 | 12 | 62 |
| AK004867 | 1300002P22Rik | 13 | 63 |
| AK015355 | 4930442A21Rik | 14 | 64 |
| AK009315 | 2310012G06Rik | 15 | 65 |
| AJ277212 | hypothetical | 16 | 66 |
| | protein | | |
| NM_026167 | 1200009K10Rik | 17 | 67 |
| NM_011782 | Adamts5 | 18 | 68 |
| NM_020578 | Ehd3 | 19 | 69 |
| NM_016873 | Wisp2 | 20 | 70 |
| AV280352 | AV280352 | 21 | 71 |
| AK010891 | 2510002J07Rik | 22 | 72 |
| AK020638 | 9530072E15Rik | 23 | 73 |
| AK018128 | 6330406I15Rik | 24 | 74 |
| AK004732 | 1200013A08Rik | 25 | 75 |
| BC004720 | MGC36388 | 26 | 76 |
| NM_026252 | 4930447D24Rik | 27 | 77 |
| NM_031180 | Klb-pending | 28 | 78 |
| NM_020025 | B3galt2 | 29 | 79 |
| AK004897 | Facl2 | 30 | 80 |
| AK016444 | 4931408D14Rik | 31 | 81 |
| AK013740 | 6530401D17Rik | 32 | 82 |
| AF090738 | Irs2 | 33 | 83 |
| | | | 84 |
| AK004293 | 2310041C05Rik | 34 | 85 |
| BC003479 | LOC216820 | 35 | 86 |
| AKO18673 | Mrpl19 | 36 | 87 |
| AB001735 | Adamts1 | 37 | 88 |
| AKO18423 | 8430417G17Rik | 38 | 89 |
| AK016103 | 4930553F04Rik | 39 | 90 |
| BC003755 | Eya2 | 40 | 91 |
| BB265432 | BB265432 | 41 | 92 |
| NM_013743 | Pdk4 | 42 | 93 |
| | | | 94 |
| U03560 | Hsp25 | 43 | 95 |
| J04632 | Gstm1 | 44 | 96 |
| L12447 | Igfbp5 | 45 | 97 |
| M21855 | Cyp2b9 | 46 | 98 |
| AI467229 | Ppp1r3a | 47 | 99 |
| X13297 | Acta2 | 48 | 100 |
| Z37107 | Ephx2 | 49 | 101 |
| AW146087 | BB104597 | 50 | 102 |

TABLE 2
PPARγ_3T3L1_Efficacy_Probe_22 (Species:
Mouse Cell Line) (A subset of Table_1:
PPARγ_Mouse_Efficacy_Probe_52 (Species: db/db Mouse)

| Accession | | Gene SEQ | Probe SEQ |
|---|---|---|---|
| number | Gene Name | ID NO | ID NO |
| AW909114 | MGC28611 | 2 | 52 |
| NM_008543 | Madh7 | 3 | 53 |
| NM_030701 | Pumag-pending | 8 | 58 |
| AK012169 | Slitl2 | 9 | 59 |
| AK009315 | 2310012G06Rik | 15 | 65 |
| AJ277212 | hypothetical protein | 16 | 66 |
| NM_011782 | Adamts5 | 18 | 68 |
| NM_020578 | Ehd3 | 19 | 69 |
| AV280352 | AV280352 | 21 | 71 |
| AK020638 | 9530072E15Rik | 23 | 73 |
| AK004732 | 1200013A08Rik | 25 | 75 |
| BC004720 | MGC36388 | 26 | 76 |
| NM_031180 | Klb-pending | 28 | 78 |
| AK013740 | 6530401D17Rik | 32 | 82 |
| BC003479 | LOC216820 | 35 | 86 |
| AB001735 | Adamts1 | 37 | 88 |
| AKO18423 | 8430417G17Rik | 38 | 89 |
| AK016103 | 4930553F04Rik | 39 | 90 |
| NM_013743 | Pdk4 | 42 | 93 |
| | | | 94 |
| J04632 | Gstm1 | 44 | 96 |
| Z37107 | Ephx2 | 49 | 101 |

Genetically altered, diabetic, mice (db/db strain, available from the Jackson Laboratory, Bar Harbor, Me., U.S.A., as strain C57B1/KFJ, and described by Chen et al., Cell 84: 491-495 (1996), and by Combs et al., Endocrinology 142: 998-1007 (2002)), and lean mice, were administered one of two PPARγ agonists, either Rosiglitazone (5-(4-{2-[methyl(pyridin-2-yl)amino]ethoxy}benzyl)-1,3-thiazolidine-2,4-dione) or {2-[2-(4-phenoxy-2-propylphenoxy)ethyl]-1H-indol-5-yl}acetic acid. The PPARγ agonists were orally administered once per day for a period of two days or eight days at a dosage of 10 milligrams per kilogram body weight. EWAT tissue was removed from the treated mice six hours after administration of the second or eighth dose. Both of the treatments were divided into four groups:

Group 1: db/db vehicle control vs. db/db vehicle control pool (the control pool included all of the mice that were administered the vehicle alone without any PPARγ agonist).

Group 2: lean mouse vs. db/db vehicle control pool.

Group 3: db/db vehicle control pool vs. Rosiglitazone-treated db/db mice.

Group 4: db/db vehicle control pool vs. db/db mice treated with {2-[2-(4-phenoxy-2-propylphenoxy)ethyl]-1H-indol-5-yl}acetic acid.

A hybrid ANOVA method was used to compute the pvalue (hereafter ANOVA-pvalue) for the null hypothesis that the genes are not differentially regulated within each group. Standard ANOVA estimates the variance within a group by the spread of replicates within each group. The error of the variance within a group can be large when the number of replicates in each group is small, thereby yielding more false positives (mistakenly identifying a non-significant difference between groups as being significant). This problem is avoided by using the hybrid ANOVA method to estimate the error within a group. The variance within a group comes from at least two sources: sample variance and measurement error (platform variance). The Hybrid-ANOVA sets a low limit of the within-group variance to the platform variance. The platform variance is estimated from previous replicates with similar gene expression levels.

Signature genes were identified for each of the four groups (i.e., genes that showed significant, differential, expression in the comparison made in each of the four groups). Based upon the two day data (each treatment was repeated five times), each probe having an ANOVA-pvalue smaller than 0.01, and having an absolute value of the mean of the logRatio greater than log₁₀ 1.5 was considered to be a signature gene for each group.

First, the signature genes in Groups 3 and 4 were united. Then the united signature genes from Groups 3 and 4 were compared with the signature genes from Group 2, and the overlapping population of genes between the two compared groups was identified. Then the genes within the overlapping population that were regulated in the opposite direction in the united signature gene population compared to the Group 2 signature gene population were identified (e.g., genes that are differentially expressed at a higher, or lower, level in the db/db mice, but are differentially expressed at a lower, or higher, level in mice treated with a PPARγ agonist are likely to be markers for the desired effect of reducing blood glucose level).

Finally, artifactual signature genes in Group 1 were removed from the resulting set. The artifactual signature genes are those genes that were differentially regulated in Group 1, and so represented the variation in gene expression between animals. A total of 52 probes (SEQ ID NOs: 51-102) were thereby identified as the efficacy reporter population in the EWAT tissue of db/db mice treated with the PPARγ agonists. These 52 probes (SEQ ID NOs: 51-102) corresponded to 50 genes (SEQ ID NOs: 1-50). These 50 genes (SEQ ID NOs: 1-50) are useful in the practice of the present invention as an efficacy-related population of genes to identify PPARγ agonists and/or PPARγ partial agonists using mouse EWAT tissue.

The usefulness of the 50 genes (SEQ ID NOs: 1-50), as an efficacy-related population of genes to identify PPARγ agonists and/or PPARγ partial agonists, was confirmed by using the data from the treatments lasting for seven days in which eight doses were administered to the animals (the first dose being administered at day zero) to determine whether the expression of the 50 genes (SEQ ID NOs: 1-50), corresponding to the 52 probes (SEQ ID NOs: 52-102), correlated with the desired biological end point (i.e., lowering of glucose concentration in blood plasma).

The reduction in the concentration of glucose in blood plasma was measured for each mouse in the study. The correlation coefficient of the logRatio of each of the 52 probes (SEQ ID NOs: 52-102) with the end point data was calculated. Probes with correlation coefficient of more than 0.5 were selected. All 52 probes (SEQ ID NOs: 52-102) were found to have a satisfa end point data.

The 52 probes (SEQ ID NOs: 52-102) were also mapped onto the gene expression profiles of mouse 3T3L1 adipocyte cells, cultured in vitro, that had been treated with either Rosiglitazone (at an effective concentration of 600 nM) or {2-[2-(4-phenoxy-2-propylphenoxy)ethyl]-1H-indol-5-yl}acetic acid (at an effective concentration of 3870 nM). Twenty four hours after the cells were contacted with one or other of the foregoing agents the cells were harvested and RNA extracted therefrom. Twenty two probes (SEQ ID NOs: 52, 53, 58, 59, 65, 66, 68, 69, 71, 73, 75, 76, 78, 82, 86, 88-90, 93, 94, 96, 101) were identified that were differentially regulated in the 3T3L1 adipocytes in response to both of the foregoing agents. These 22 probes (SEQ ID NOs: 52, 53, 58, 59, 65, 66, 68, 69, 71, 73, 75, 76, 78, 82, 86, 88-90, 93, 94, 96, 101) corresponded to 21 genes (two probes hybridized to the same gene) (SEQ ID NOs: 2, 3, 8, 9, 15, 16, 18, 19, 21, 23, 25, 26, 28, 32, 35, 37-39, 42, 44, 49). These 21 genes (SEQ ID NOs: 2, 3, 8, 9, 15, 16, 18, 19, 21, 23, 25, 26, 28, 32, 35, 37-39, 42, 44, 49) are useful in the practice of the present invention as an efficacy-related population of genes to identify PPARγ agonists and/or PPARγ partial agonists using the 3T3L1 mouse cell line.

The expression data for the 21 genes (SEQ ID NOs: 2, 3, 8, 9, 15, 16, 18, 19, 21, 23, 25, 26, 28, 32, 35, 37-39, 42, 44, 49) in response to Rosiglitazone and PPARγ agonist {2-[2-(4-phenoxy-2-propylphenoxy)ethyl]-1H-indol-5-yl}acetic acid were averaged and treated as a vector for the full template. Thus, an efficacy value a PPARγ agonist, or partial agonist, was calculated in the following manner. The value (expressed as a percentage) of the logRatio divided by the template logRatio for each of the 22 probes (SEQ ID NOs: 52, 53, 58, 59, 65, 66, 68, 69, 71, 73, 75, 76, 78, 82, 86, 88-90, 93, 94, 96, 101) was calculated, and then the mean of the resulting 22 percentages was calculated. This mean value was the PPARγ efficacy value for the PPARγ agonist, or partial agonist.

A chi-square fitting was also used to calculate the efficacy value for each tested PPARγ agonist, or partial agonist. The chi-square fitting formula used was:

$${χ}^{2} = {\sum }_{i = 1}^{22} {( S * {R}_{i} - {X}_{i} )}^{2} / ( {σ}_{Ri}^{2} + {σ}_{Xi}^{2} )$$

Where Ri, σ_{Ri} stand for the logRatio and error for logRatio of the full template. Xi and σ_{Xi} stand for the logRatio and error for logRatio of the testing compound. This chi-square fitting method is described, for example, by W. Press et al., Numerical Recipes in C, Chapter 14, Cambridge University Press (1991).

A very similar result was obtained using each method for calculating the efficacy values (the correlation coefficient for the scores calculated by the two methods was 0.9996).

Table 3 shows the efficacy scores for full or partial agonists of PPARγ. A PPARα agonist was included as a control.

TABLE 3

| Compound | Efficacy Score |
|---|---|
| Agonist 1 | 1.033 |
| Agonist | 0.967 |
| Rosiglitazone | |
| Partial agonist 15 | 0.795 |
| Partial agonist 16 | 0.776 |
| Partial agonist 17 | 0.644 |
| Partial agonist 4 | 0.578 |
| Partial agonist (2R)-2-(4-chloro-3-{[3- | 0.561 |
| (6-methoxy-1,2-benzisoxazol-3-yl)-2-methyl- | |
| 6-(trifluoromethoxy)-1H-indol-1- | |
| yl]methyl}phenoxy)propanoate | |
| Partial agonist 10 | 0.511 |
| Partial agonist 12 | 0.469 |
| Partial agonist 9 | 0.463 |
| Partial agonist 11 | 0.447 |
| Partial agonist 14 | 0.376 |
| Partial agonist 13 | 0.367 |
| PPARα agonist | 0.178 |

### EXAMPLE 2

This Example describes the identification of toxicity-related populations of genes that are useful in the practice of the methods of the invention for evaluating the toxic, or otherwise undesirable, biological activities of agonists and partial agonists of PPARγ.

Measuring the Toxic Effects of PPARγ Agonists and PPARγ Partial Agonists in Rats: Eleven PPARγ agonists or partial agonists were tested in rats in an experiment that was divided into several experiments (referred to as phases) because the design of the overall experiment required the use of more rats than could be handled in a single experiment. Each phase of the experiment tested 3 compounds, with rosiglitazone present in every phase as a bridging compound. For each compound, 3 doses were selected that represented the effective dose (EC₅₀) in db/db mice, as well as ⅓ and 3 times the EC₅₀. Eight animals were treated per dose and per compound. The treatments lasted 7 days, and a PPARγ agonist or partial agonist was administered once per day. Animals were sacrificed 24 hours, or later, after the last dose of the treatment, so that the plasma volume data could be measured. Heart, kidney and EWAT tissues from phases 5, 7, 8 and 9 were collected. For phase 4, only heart tissues were available. Heart weight, body weight and plasma volume data were recorded for each animal.

Microarray profiling: Heart, kidney and EWAT tissues were profiled using gene microarrays to identify genes that are toxicity biomarkers. Tissues from the animals treated only with the vehicle (that did not include a PPARγ agonist or partial agonist) were used as the reference channel for the microarray profiling. cDNA made from RNA extracted from tissues from animals treated with a PPARγ agonist, or partial agonist, were labeled with different fluorophores and competitively hybridized with the reference sample on the same array. Approximately 25,000 rat genes had representative oligonucleotide probes on the array. To save the array budget, only a subset of animals were profiled for some phases. When selecting the subset of animals for profiling, efforts were made to avoid biases by choosing animals covering a broad range of biological endpoints. In those phases where a subset were selected, 3 out of 8 rats were selected from the low and medium dose, 6 out of 8 rats were selected from the high dose. It was assumed that effects associated with the high dose were more likely to be drug effects.

Methods for Identifying Toxicity-Related Genes: Genes were selected whose expression correlated with heart weight increase and/or plasma volume expansion. A dimension reduction approach was also taken to address the statistical overfitting problem. Since there were 25,000 probes printed on the microarray, it was possible to mistakenly select a few genes, by chance, whose expression appeared to be correlated with the biological end point of interest. This is referred to as the overfitting problem. The following approach was used to address the overfitting problem. Regulated genes were identified by first identifying robust signature genes for each compound (i.e., genes whose expression was consistently affected by the compound being tested). The union of the signature genes for all of the compounds tested was clustered into subgroups, and the groups of genes whose expression pattern correlated with the biological endpoint were identified. Since the number of subgroups was usually small (around 4 subgroups), there was no danger of overfitting. This Example describes application of these methods to identifying genes that are markers for increased heart weight in response to a PPARγ agonist or partial agonist.

(1) Correlating an Increase in Heart Weight with the Expression of Individual Genes in Rat Hearts: Data sets used to identify the correlation were from phases 5, 7, and 8. Gene expression was correlated with an increase in heart weight observed in rats by selecting genes significantly regulated (P<0.01) in more than 3 experiments in each data set. These genes were called the signature genes. The correlation between the log(ratio) of each of the signature genes and the increase in heart weight were calculated for each data set. In this experiment the heart weight was normalized to the body weight. Since the data set for phases 7 and 8 were relatively small, phase 7 data and phase 8 data were also combined for the above calculations, in addition to being used separately. Signature genes were selected that had a magnitude of correlation greater than 0.3 from each data set.

There were almost no overlapping genes from more than four data sets when the individual animal heart weight data was used. To reduce possible heart weight data measurement error, and to emphasize the drug related toxicity effect, the heart weight data from eight animals (irrespective of whether the animals had been profiled using the microarray) of each treatment group were averaged and used as the toxicity measurement. Using the average endpoint data, 10 overlapping genes were identified.

Since the magnitude of correlation threshold of 0.3 was arbitrary, and the number of overlapping genes was relatively small, the overlapping genes were used as the seed genes to identify similarly regulated genes in data from phases 5 and the combination of phases 7 plus 8. Genes whose regulation correlated with any of the 10 overlapping genes in either the data from phase 5 or the data from the combination of phases 7 plus 8, with a magnitude of correlation greater than 0.8, were selected. Sixty three probes were thereby identified as toxicity-related genes that indicate an undesirable increase in heart weight.

It was possible just by chance to incorrectly select a few toxicity-related genes since there were 25,000 genes present on the microarray. Therefore it was important to have some test data sets (which were not involved in the toxicity-related gene selection) to validate the toxicity-related genes.

(2) Using Strongly Regulated Genes to Identify a Toxicity Related Gene Population: Selecting toxicity-related genes based on the analysis of individual signature gene expression patterns was the most sensitive method to identify a toxicity-related gene population, but also had the highest risk of over-fitting, because of the high degree of freedom. The statistical significance was discounted by the big Bonferroni correction factor. The separate experiments were not fully independent from each other, since a bridging compound was used (rosiglitazone). Therefore a dimension reduction was used to reduce the risk of over-fitting.

First, robust signature genes (i.e., genes whose expression was consistently affected by the compound being tested and which correlated with the target biological effect) were identified in response to each PPARγ agonist, or partial agonist (P<0.01 and amplitude of log(ratio)>0.15 in at least 80% of the replicates of any treatment, same direction of regulation across multiple doses within a drug, but not in any of the control experiments with log(ratio)>0.2). Then the union of drug signature genes from each phase was analyzed to identify the signature genes that appear in more than one phase. The signature genes from all phases were clustered into a finite number of patterns (<10), and the patterns associated with increased heart weight were identified. The heart tissues from phases 5, 7, 8, 9 were used for selecting the robust signature genes.

A total of 114 signature genes were selected from all phases. Gene dimension clustering showed that two groups of genes (one up-regulated and one down-regulated) correlated with increased heart weight. The degree of the correlation of these two groups of genes with increased heart weight was further verified by calculating the correlation coefficient between the mean log(ratio) of the up-regulated (or down-regulated) group with the heart weight. The correlations were 0.75 or higher. The chance probability of having such high correlation by random fluctuation was at the level of 2×10⁻⁷.

Combining the Results of the Gene Expression Analysis Described in Sections (1) and (2): A set of 48 probes were selected from the 114 probes identified in Section (2). Combining these 48 probes with the 63 probes identified as described in Section (1) yielded a total of 85 unique probes. These probes were screened again to identify those probes having a correlation coefficient between gene expression and increase in heart weight greater than 0.4. This process resulted in the final 55 probes. The nucleotide sequence identification numbers of these 55 probes are identified in Table 4, (SEQ ID NOs: 153-207). These 55 probes (SEQ ID NOs: 153-207) corresponded to 50 different genes. The nucleotide sequence identification numbers of these 50 genes are identified in Table 4, (SEQ ID NOs: 103-152). These 50 genes (SEQ ID NOs: 103-152) are useful in the practice of the present invention as a toxicity-related gene population.

TABLE 4
PPARγ_Rat_Heart_Toxicity_HeartWeight_Probe_55
(Species: Rat)

| Accession | | Gene SEQ | Probe SEQ |
|---|---|---|---|
| number | Gene Name | ID NO | ID NO |
| AB011365 | Pparg | 103 | 153 |
| | | | 154 |
| D16478 | Hadha | 104 | 155 |
| J02791 | Acadm | 105 | 156 |
| | | | 157 |
| Y09333 | Mte1 | 106 | 158 |
| AI230591 | g3814478 | 107 | 159 |
| AI105094 | g3709266 | 108 | 160 |
| AA891470 | g3708538 | 109 | 161 |
| AI059241 | g3333018 | 110 | 162 |
| G3638603 | g3638603 | 111 | 163 |
| AA859032 | g2948383 | 112 | 164 |
| BF288765 | g3726475 | 113 | 165 |
| AI071468 | g3397683 | 114 | 166 |
| G3817698 | g3817698 | 115 | 167 |
| AI070283 | Pcsk4 | 116 | 168 |
| G3189597 | g3189597 | 117 | 169 |
| g3815735 | g3815735 | 118 | 170 |
| AI170067 | g3710107 | 119 | 171 |
| AI407765 | g3707790 | 120 | 172 |
| AI170387 | g3710427 | 121 | 173 |
| AI231193 | g3815073 | 122 | 174 |
| g979428 | g979428 | 123 | 175 |
| G3105928 | g3105928 | 124 | 176 |
| AI411979 | g3072442 | 125 | 177 |
| 600523591R1 | 600523591R1 | 126 | 178 |
| AA964752 | g3138244 | 127 | 179 |
| AI009219 | g3223051 | 128 | 180 |
| BE101435 | g2937230 | 129 | 181 |
| AI044576 | g3291437 | 130 | 182 |
| G3036695 | g3036695 | 131 | 183 |
| BG372920 | g3189161 | 132 | 184 |
| AI105417 | g3709501 | 133 | 185 |
| AI177360 | g3727998 | 134 | 186 |
| G3189544 | g3189544 | 135 | 187 |
| AI227820 | Mgll | 136 | 188 |
| AA892864 | Mgll | 137 | 189 |
| BF395162 | g3223602 | 138 | 190 |
| G977669 | g977669 | 139 | 191 |
| g4135065 | g4135065 | 140 | 192 |
| M23601 | Maob | 141 | 193 |
| L23108* | Cd36 | 142 | 194 |
| U75581 | Fabp4 | 143 | 195 |
| | | | 196 |
| | | | 197 |
| NM_012778 | Aqp1 | 144 | 198 |
| U41453 | Akap12 | 145 | 199 |
| U67863 | Mc4r | 146 | 200 |
| | | | 201 |
| NM_031315 | Cte1 | 147 | 202 |
| NM_013120 | Gckr | 148 | 203 |
| NM_017306 | Dci | 149 | 204 |
| NM_022594 | Ech1 | 150 | 205 |
| D00729 | D00729 | 151 | 206 |
| NM_021751 | Prom | 152 | 207 |

*Mouse gene sequence L23108 (SEQ ID NO: 142) and corresponding mouse probe (SEQ ID NO: 194) were used to measure gene expression of the rat homolog(s) to mouse Cd36 gene.

Identifying a Toxicity-Related Gene Population in Mice that are Early Predictors for Increased Heart Weight: The 55 probes (SEQ ID NOs: 153-207) corresponding to the toxicity-related population of 50 genes (SEQ ID NOs: 103-152), described in the preceding paragraph, were further analyzed to identify a sub-population of genes that are useful as early biomarkers for the onset of the adverse effect of heart weight increase due to administration of a PPARγ agonist or partial agonist.

In order to find the early biomarkers, the 55 probes (SEQ ID NOs: 153-207) were mapped onto an earlier data set, obtained by treating mice with PPARγ agonists and partial agonists. This earlier experiment was referred to as the “747 tissue experiment” since 747 tissues were collected. PPARγ agonists Rosiglitazone and 5-[4-(3-{4-[4-(methyl sulfonyl)phenoxy]-2-propylphenoxy}propoxy)phenyl]-1,3-thiazolidine-2,4-dione were administered to mice once per day for one to seven days. Tissues were removed 6 hours after the most recent dose of PPARγ agonist from animals with 1, 2, 4 and 8 treatments (note that the first dosage was administered at time zero and tissues were removed from the treated animals six hours later; thus, the animals sacrificed at 7 days had received 8 treatments). By mapping the 55 rat probes (SEQ ID NOs: 153-207) into this set of mice data, and also requiring genes to be regulated by just one or two treatments, five early biomarkers were identified that were useful early reporters of heart toxicity. The nucleotide sequences of these 6 probes (SEQ ID NOs: 213-218), corresponding to 5 genes (SEQ ID NOs: 208-212), as identified in Table 5.

TABLE 5
PPARγ_Mouse_Heart_EarlyBiomarkers_ForHeartWeight_{—}
Probe_5 (species Mouse)

| Accession | | Gene SEQ | Probe SEQ |
|---|---|---|---|
| number | Gene Name | ID NO | ID NO |
| AK003305 | 1110002J19Rik | 208 | 213 |
| AJ001118 | Mgll | 209 | 214 |
| M13264 | Fabp4 | 210 | 215 |
| | | | 216 |
| L02914 | Aqp1 | 2ll | 217 |
| U01841 | Pparg | 212 | 218 |

These early biomarkers are also useful as a toxicity-related gene population in the practice of the present invention. The use of these early biomarkers helps to identify those candidate PPARγ agonists and/or partial agonists that possess the undesirable property of causing an increase in heart weight.

Heart Weight Biomarkers in EWAT: EWAT is a target tissue for the PPARγ agonists, and is a useful tissue for microarray profiling because it has a high signal to noise ratio. In addition, it is advantageous to be able to assess both efficacy and toxicity using the same tissue.

Approximately 1800 robust signature genes were selected (using data from phases 5, 7, 8 and 9). The log(ratio)s of the 1800 robust EWAT signature genes were directly correlated with heart weight. 355 Probes were identified, from the population of 1800 robust probes, that had a correlation value of at least 0.6. The correlation value was a measure of correlation between expression of the gene corresponding to the probe and an increase in heart weight. The identities of these 355 probes are given in Table 6 (SEQ ID NOs: 551-894, 155, 157, 164, 171, 178, 179, 185, 188, 204-206). These 355 probes (SEQ ID NOs: 551-894, 155, 157, 164, 171, 178, 179, 185, 188, 204-206) corresponded to 343 different genes that are identified in Table 6 (SEQ ID NOs: 219-550, 104, 105, 112, 119, 126, 127, 133, 136, 149-151).

TABLE 6
PPARγ_Rat_eWAT_Toxicity_HeartWeight_Probe_355
(Species: Rat)

| Accession | | Gene SEQ | Probe SEQ |
|---|---|---|---|
| number | Gene Name | ID NO | ID NO |
| AA956114 | | 219 | 551 |
| D00688 | Maoa | 220 | 552 |
| | | | 553 |
| D16478 | Hadha | 104 | 155 |
| J02791 | Acadm | 105 | 157 |
| J05029 | Acadl | 221 | 554 |
| | | | 555 |
| | | | 556 |
| K03249 | Ehhadh | 222 | 557 |
| | | | 558 |
| | | | 559 |
| M22756 | Ndufv2 | 223 | 560 |
| M29853 | Cyp4b1 | 224 | 561 |
| | | | 562 |
| | | | 563 |
| G3292626 | g3292626 | 225 | 564 |
| AI170251 | g3710291 | 226 | 565 |
| AI411835 | g3019978 | 227 | 566 |
| AI229166 | g3813053 | 228 | 567 |
| G3667853 | g3667853 | 229 | 568 |
| AA891248 | g3018127 | 230 | 569 |
| G3731024 | g3731024 | 231 | 570 |
| BF282327 | g3812938 | 232 | 571 |
| AA944463 | g3104379 | 233 | 572 |
| G3704882 | g3704882 | 234 | 573 |
| AI113016 | g3512965 | 235 | 574 |
| AW142276 | g3815698 | 236 | 575 |
| G3103828 | g3103828 | 237 | 576 |
| 700034842H1 | 700034842H1 | 238 | 577 |
| AI408705 | g2863227 | 239 | 578 |
| G3227498 | g3227498 | 240 | 579 |
| G3291499 | g3291499 | 241 | 580 |
| AI030918 | g3248744 | 242 | 581 |
| G3712254 | g3712254 | 243 | 582 |
| G3728605 | g3728605 | 244 | 583 |
| G979167 | g979167 | 245 | 584 |
| G3189034 | g3189034 | 246 | 585 |
| G3018667 | g3018667 | 247 | 586 |
| G3188003 | g3188003 | 248 | 587 |
| AI170000 | g3710040 | 249 | 588 |
| X57405 | Notch1 | 250 | 589 |
| G979644 | g979644 | 251 | 590 |
| G3712007 | g3712007 | 252 | 591 |
| AI144876 | Ass | 253 | 592 |
| AI235475 | g3828981 | 254 | 593 |
| AW915407 | g2938925 | 255 | 594 |
| BF288349 | g2938279 | 256 | 595 |
| AI228128 | g3812015 | 257 | 596 |
| AI411031 | g3709121 | 258 | 597 |
| AI168968 | g3705276 | 259 | 598 |
| BF398271 | g3292264 | 260 | 599 |
| G2862965 | g2862965 | 261 | 600 |
| G807326 | g807326 | 262 | 601 |
| G4133385 | g4133385 | 263 | 602 |
| BE107150 | g2939171 | 264 | 603 |
| AI044760 | g3291621 | 265 | 604 |
| BF400209 | g3226969 | 266 | 605 |
| G3705573 | g3705573 | 267 | 606 |
| BF283751 | g4132683 | 268 | 607 |
| AI411520 | g4134016 | 269 | 608 |
| BF560807 | g3187199 | 270 | 609 |
| G3221992 | g3221992 | 271 | 610 |
| G4131482 | g4131482 | 272 | 611 |
| G3071873 | g3071873 | 273 | 612 |
| AA799476 | g2862431 | 274 | 613 |
| G977129 | g977129 | 275 | 614 |
| g3399275 | g3399275 | 276 | 615 |
| G3729761 | g3729761 | 277 | 616 |
| AI411212 | g3710380 | 278 | 617 |
| AI180004 | g3730642 | 279 | 618 |
| AI411375 | g2939160 | 280 | 619 |
| G3223977 | g3223977 | 281 | 620 |
| BE116768 | g3638204 | 282 | 621 |
| BF282695 | g3511588 | 283 | 622 |
| 701347850H1 | 701347850H1 | 284 | 623 |
| G3709587 | g3709587 | 285 | 624 |
| G3813131 | g3813131 | 286 | 625 |
| AI603127 | g3222358 | 287 | 626 |
| G3223106 | g3223106 | 288 | 627 |
| AA859032 | g2948383 | 112 | 164 |
| G3225430 | g3225430 | 289 | 628 |
| G3019722 | g3019722 | 290 | 629 |
| g3292396 | g3292396 | 291 | 630 |
| AI599484 | g3119754 | 292 | 631 |
| BE110616 | g3726615 | 293 | 632 |
| G3187488 | g3187488 | 294 | 633 |
| AI044912 | g3291731 | 295 | 634 |
| AI511066 | g3667675 | 296 | 635 |
| AA891689 | g3018568 | 297 | 636 |
| AA799829 | g4131444 | 298 | 637 |
| AI101639 | g3706514 | 299 | 638 |
| AI013110 | g3227166 | 300 | 639 |
| G3019363 | g3019363 | 301 | 640 |
| g3636884 | g3636884 | 302 | 641 |
| BF284475 | g3711260 | 303 | 642 |
| AA894090 | g3020969 | 304 | 643 |
| G2863149 | g2863149 | 305 | 644 |
| G977018 | g977018 | 306 | 645 |
| BE113034 | g3815452 | 307 | 646 |
| G3137782 | g3137782 | 308 | 647 |
| 700064632H1 | 700064632H1 | 309 | 648 |
| G3292491 | g3292491 | 310 | 649 |
| AI599819 | g3120109 | 311 | 650 |
| AI233766 | g3817646 | 312 | 651 |
| 700508236H1 | 700508236H1 | 313 | 652 |
| 701347935H1 | 701347935H1 | 314 | 653 |
| g2937470 | g2937470 | 315 | 654 |
| AI170808 | g3710848 | 316 | 655 |
| G3727129 | g3727129 | 317 | 656 |
| AW528443 | g4136134 | 318 | 657 |
| AI235135 | g3828641 | 319 | 658 |
| G3511674 | g3511674 | 320 | 659 |
| BG372437 | g4135897 | 321 | 660 |
| BF556962 | g3708808 | 322 | 661 |
| AI144760 | g3666559 | 323 | 662 |
| AI598414 | g3396210 | 324 | 663 |
| g3118749 | g3118749 | 325 | 664 |
| AI511051 | g3511894 | 326 | 665 |
| AA963069 | g3136561 | 327 | 666 |
| G3729474 | g3729474 | 328 | 667 |
| G3709332 | g3709332 | 329 | 668 |
| BF288286 | g2937985 | 330 | 669 |
| AI170067 | g3710107 | 119 | 171 |
| AI175045 | g3725683 | 331 | 670 |
| BG373072 | g3816835 | 332 | 671 |
| BF405032 | g3035182 | 333 | 672 |
| G4134345 | g4134345 | 334 | 673 |
| BG373122 | g978418 | 335 | 674 |
| BG381583 | g4132471 | 336 | 675 |
| G2863503 | g2863503 | 337 | 676 |
| BF281235 | g3121225 | 338 | 677 |
| AA892281 | g3019160 | 339 | 678 |
| AI168935 | g4134349 | 340 | 679 |
| G3223313 | g3223313 | 341 | 680 |
| AA998205 | g3188856 | 342 | 681 |
| G3705112 | g3705112 | 343 | 682 |
| AA799656 | g2862611 | 344 | 683 |
| 701219674H1 | 701219674H1 | 345 | 684 |
| G3103230 | g3103230 | 346 | 685 |
| AA998461 | g3189112 | 347 | 686 |
| BG378631 | g3729576 | 348 | 687 |
| AW525026 | g3246829 | 349 | 688 |
| AA964882 | g3138374 | 350 | 689 |
| G3513255 | g3513255 | 351 | 690 |
| AI009759 | g3223591 | 352 | 691 |
| BG378729 | g3104259 | 353 | 692 |
| BF283386 | g3121114 | 354 | 693 |
| AW915566 | g2864131 | 355 | 694 |
| BF288366 | g2938368 | 356 | 695 |
| g2864124 | g2864124 | 357 | 696 |
| 701216507H1 | 701216507H1 | 358 | 697 |
| G2937254 | g2937254 | 359 | 698 |
| AA892593 | g3019472 | 360 | 699 |
| BG377008 | g2863410 | 361 | 700 |
| AI231886 | g3815766 | 362 | 701 |
| AI406687 | g3019436 | 363 | 702 |
| AI137895 | g3638672 | 364 | 703 |
| BF558361 | g3706834 | 365 | 704 |
| AI060312 | g3334089 | 366 | 705 |
| AI058968 | g3332745 | 367 | 706 |
| 701349156H1 | 701349156H1 | 368 | 707 |
| 700032770H1 | 700032770H1 | 369 | 708 |
| 701220604H1 | 701220604H1 | 370 | 709 |
| 701222864H1 | 701222864H1 | 371 | 710 |
| 701218584H1 | 701218584H1 | 372 | 711 |
| 700508607H1 | 700508607H1 | 373 | 712 |
| G979526 | g979526 | 374 | 713 |
| 600507145R1 | 600507145R1 | 375 | 714 |
| 600513733R1 | 600513733R1 | 376 | 715 |
| 600521564R1 | 600521564R1 | 377 | 716 |
| G979217 | g979217 | 378 | 717 |
| 600521930R1 | 600521930R1 | 379 | 718 |
| 600511860R1 | 600511860R1 | 380 | 719 |
| 600512417R1 | 600512417R1 | 381 | 720 |
| 701417945H1 | 701417945H1 | 382 | 721 |
| 600516384R1 | 600516384R1 | 383 | 722 |
| G3711582 | g3711582 | 384 | 723 |
| 600516355R1 | 600516355R1 | 385 | 724 |
| 600511327R1 | 600511327R1 | 386 | 725 |
| AI600147 | 600521079R1 | 387 | 726 |
| G4134738 | g4134738 | 388 | 727 |
| G3727115 | g3727115 | 389 | 728 |
| 600521206R1 | 600521206R1 | 390 | 729 |
| AA819547 | g2889636 | 391 | 730 |
| BF281400 | g2672900 | 392 | 731 |
| 600523591R1 | 600523591R1 | 126 | 178 |
| 600521690R1 | 600521690R1 | 393 | 732 |
| 600510887R1 | 600510887R1 | 394 | 733 |
| AI175980 | 600512928R1 | 395 | 734 |
| AA944036 | g3103952 | 396 | 735 |
| 600518269R1 | 600518269R1 | 397 | 736 |
| AI175479 | 600513115R1 | 398 | 737 |
| G3188371 | g3188371 | 399 | 738 |
| 700692105H1 | 700692105H1 | 400 | 739 |
| G3225638 | g3225638 | 401 | 740 |
| 600507783R1 | 600507783R1 | 402 | 741 |
| S74321 | cytochrome bc-l | 403 | 742 |
| | complex core P | | |
| BE109568 | 600509475R1 | 404 | 743 |
| G3071118 | g3071118 | 405 | 744 |
| AI010433 | Cdtwl | 406 | 745 |
| G2938798 | g2938798 | 407 | 746 |
| AA866477 | g2961938 | 408 | 747 |
| BG381033 | g4131620 | 409 | 748 |
| 600512426R1 | 600512426R1 | 410 | 749 |
| 600509794R1 | 600509794R1 | 411 | 750 |
| G2862597 | g2862597 | 412 | 751 |
| XM341383 | Pcca | 413 | 752 |
| AI228236 | g3812123 | 414 | 753 |
| 600512874R1 | 600512874R1 | 415 | 754 |
| G4134262 | g4134262 | 416 | 755 |
| 600523104R1 | 600523104R1 | 417 | 756 |
| 600520906R1 | 600520906R1 | 418 | 757 |
| G4131829 | g4131829 | 419 | 758 |
| AI231810 | g3815690 | 420 | 759 |
| AI072712 | 600507095R1 | 421 | 760 |
| 600515268R1 | 600515268R1 | 422 | 761 |
| G3815486 | g3815486 | 423 | 762 |
| 600509881R1 | 600509881R1 | 424 | 763 |
| AI232494 | g3816374 | 425 | 764 |
| AA964752 | g3138244 | 127 | 179 |
| AI410548 | g3073005 | 426 | 765 |
| G3104296 | g3104296 | 427 | 766 |
| 600514084R1 | 600514084R1 | 428 | 767 |
| 600519478R1 | 600519478R1 | 429 | 768 |
| 600508574R1 | 600508574R1 | 430 | 769 |
| AA875107 | g2980055 | 431 | 770 |
| AI104528 | g3708870 | 432 | 771 |
| G3227353 | g3227353 | 433 | 772 |
| AI171656 | g3711696 | 434 | 773 |
| G2863419 | g2863419 | 435 | 774 |
| BE102621 | g3512812 | 436 | 775 |
| G3398286 | g3398286 | 437 | 776 |
| g3830855 | g3830855 | 438 | 777 |
| AI104348 | g3708719 | 439 | 778 |
| AI599410 | g2889576 | 440 | 779 |
| G3831232 | g3831232 | 441 | 780 |
| AI145507 | g3667306 | 442 | 781 |
| G3396295 | g3396295 | 443 | 782 |
| AA891814 | g3018693 | 444 | 783 |
| G4133678 | g4133678 | 445 | 784 |
| AW434257 | g3397092 | 446 | 785 |
| G3019879 | g3019879 | 447 | 786 |
| G3018575 | g3018575 | 448 | 787 |
| AI412460 | g3704629 | 449 | 788 |
| BG381624 | g3018621 | 450 | 789 |
| AW142969 | g3727595 | 451 | 790 |
| G978652 | g978652 | 452 | 791 |
| AI105417 | g3709501 | 133 | 185 |
| AI072493 | g3398687 | 453 | 792 |
| G2862397 | g2862397 | 454 | 793 |
| AA800782 | g4131537 | 455 | 794 |
| AI171367 | g3711407 | 456 | 795 |
| BE111132 | g3397248 | 457 | 796 |
| G977490 | g977490 | 458 | 797 |
| 700585804H1 | 700585804H1 | 459 | 798 |
| BF288776 | g3726534 | 460 | 799 |
| G4135910 | g4135910 | 461 | 800 |
| G979011 | g979011 | 462 | 801 |
| BG374035 | g3726504 | 463 | 802 |
| G978793 | g978793 | 464 | 803 |
| G3707669 | g3707669 | 465 | 804 |
| 701350526H1 | 701350526H1 | 466 | 805 |
| 701216526H1 | 701216526H1 | 467 | 806 |
| AI227820 | Mgll | 136 | 188 |
| BE103080 | g3811971 | 468 | 807 |
| G3666755 | g3666755 | 469 | 808 |
| G3728883 | g3728883 | 470 | 809 |
| G4132495 | g4132495 | 471 | 810 |
| AI011448 | g4133423 | 472 | 811 |
| AI230746 | g3814633 | 473 | 812 |
| AW253370 | g3104091 | 474 | 813 |
| AA965106 | g3138598 | 475 | 814 |
| AI009609 | g4133075 | 476 | 815 |
| BG372547 | g3019278 | 477 | 816 |
| G4135366 | g4135366 | 478 | 817 |
| D50306 | Slc15al | 479 | 818 |
| D30035 | Prdx1 | 480 | 819 |
| | | | 820 |
| M63837 | Pdgfra | 481 | 821 |
| J02749 | Acaa | 482 | 822 |
| | | | 823 |
| X05341 | Acaa2 | 483 | 824 |
| M22631 | Pcca | 484 | 825 |
| L11276 | Acadl | 485 | 554 |
| | | | 555 |
| | | | 556 |
| D16479 | Hadhb | 486 | 826 |
| NM_017005 | Fh | 487 | 827 |
| NM_012891 | Acadvl | 488 | 828 |
| AF160978 | Ly68 | 489 | 829 |
| U40652 | Ptprn | 490 | 830 |
| X68101 | trg | 491 | 831 |
| NM_022398 | LOC64201 | 492 | 832 |
| NM_019274 | Colq | 493 | 833 |
| NM_024360 | Hes1 | 494 | 834 |
| AF034577 | Pdk4 | 495 | 835 |
| AF139830 | Igfbp-5 | 496 | 836 |
| AB047541 | Idh3a | 497 | 837 |
| NM_022503 | Cox7a3 | 498 | 838 |
| D10041 | Facl6 | 499 | 839 |
| AB028626 | Rasa3 | 500 | 840 |
| AJ245619 | Ctl1 | 501 | 841 |
| NM_022540 | Prdx3 | 502 | 842 |
| NM_012817 | Igfbp5 | 503 | 843 |
| NM_031032 | Gmfb | 504 | 844 |
| NM_032614 | Txnl2 | 505 | 845 |
| NM_019147 | Jag1 | 506 | 846 |
| NM_012966 | Hspe1 | 507 | 847 |
| M22030 | ETF | 508 | 848 |
| X61106 | Pgy4 | 509 | 849 |
| NM_012839 | Cycs | 510 | 850 |
| AB047540 | IDH3B | 511 | 851 |
| NM_022395 | Pmpcb | 512 | 852 |
| AJ277747 | Masp2 | 513 | 853 |
| NM_024392 | Hsd17b4 | 514 | 854 |
| NM_031511 | Igf2 | 515 | 855 |
| NM_033349 | Hagh | 516 | 856 |
| NM_031510 | Idh1 | 517 | 857 |
| NM_017267 | Timm44 | 518 | 858 |
| D50664 | Slc15a1 | 519 | 859 |
| NM_012985 | Ndufa5 | 520 | 860 |
| NM_031645 | Ramp1 | 521 | 861 |
| NM_024139 | Chp | 522 | 862 |
| AJ271158 | LOC171069 | 523 | 863 |
| AF150082 | Timm8a | 524 | 864 |
| NM_031354 | Vdac2 | 525 | 865 |
| NM_017306 | Dci | 149 | 204 |
| NM_022594 | Ech1 | 150 | 205 |
| NM_017092 | Tyro3 | 526 | 866 |
| AB032178 | Cox17 | 527 | 867 |
| X56228 | Tst | 528 | 868 |
| NM_032615 | Mir16 | 529 | 869 |
| X05634 | Sod1 | 530 | 870 |
| | | | 871 |
| | | | 872 |
| AJ245707 | Hpcl2 | 531 | 873 |
| J03621 | Suclg1 | 532 | 874 |
| NM_019187 | Coq3 | 533 | 875 |
| NM_024001 | RPT | 534 | 876 |
| NM_019278 | Resp18 | 535 | 877 |
| X97831 | Slc25a20 | 536 | 878 |
| NM_017283 | Psma6 | 537 | 879 |
| NM_031821 | Snk | 538 | 880 |
| AF095449 | Hadhsc | 539 | 881 |
| M89902 | Bdh | 540 | 882 |
| D00729 | D00729 | 151 | 206 |
| AB041723 | Pdcd8 | 541 | 883 |
| AF285103 | Psmb7 | 542 | 884 |
| NM_031851 | Phb | 543 | 885 |
| NM_031350 | Pex3 | 544 | 886 |
| NM_024386 | Hmgcl | 545 | 887 |
| L14684 | EF-G | 546 | 888 |
| U88295 | Cpt2 | 547 | 889 |
| | | | 890 |
| | | | 891 |
| AF239219 | Slc21a11 | 548 | 892 |
| M64780 | Agrn | 549 | 893 |
| AJ007704 | Mlycd | 550 | 894 |

Mapping the 355 Rat Probes (SEQ ID NOs: 551-894, 155, 157, 164, 171, 178, 179, 185, 188, 204-206) to Mouse 3T3L1 Cells in Culture: Since the 3T3L1 is a mouse cell line, the 355 EWAT probes (SEQ ID NOs: 551-894, 155, 157, 164, 171, 178, 179, 185, 188, 204-206) from rat were mapped to mouse homologs. The mapped mouse probes were then checked in the 3T3L1 PPARγ experiments (as described in Example 3) for regulation. There were 74 probes corresponding to 57 genes which were regulated with magnitude of log(ratio) greater than 0.2 (and P-value of regulation less than 1% in more than 3 experiments) in response to a PPARγ agonist or partial agonist. These 57 genes are useful in the practice of the present invention as a toxicity-related population of genes. The nucleotide sequence identification numbers of these 74 probes are identified in Table 7, (SEQ ID NOs: 950-1019, 863, 93, 94, 97). These 74 probes (SEQ ID NOs: 950-1019, 863, 93, 94, 97) corresponded to 57 different genes. The nucleotide sequence identification numbers of these 57 genes identified in Table 7, (SEQ ID NOs: 895-949, 42, 45).

TABLE 7
PPARγ_3T3L1_Toxicity_HeartWeight_Probe_74
(Species: Mouse Cell Line)

| | | Gene | Probe |
|---|---|---|---|
| Accession | | SEQ | SEQ |
| number | Gene Name | ID NO | ID NO |
| AK003953 | Tst | 895 | 950 |
| AK013511 | Ndufv2 | 896 | 951 |
| AK004125 | 1110036H20Rik | 897 | 952 |
| AK005084 | Ndufa4 | 898 | 953 |
| AF412297 | Ghitm | 899 | 954 |
| NM_026179 | 1300003D03Rik | 900 | 955 |
| AK007415 | 1810010A06Rik | 901 | 956 |
| NM_025384 | 1110003P16Rik | 902 | 957 |
| AK008511 | Usmg5 | 903 | 863 |
| AK018763 | Agt | 904 | 958 |
| BC004045 | LOC212442 | 905 | 959 |
| AK005067 | Chp-pending | 906 | 960 |
| AB047323 | COX17 | 907 | 961 |
| AK002483 | 0610010I20Rik | 908 | 962 |
| AK004390 | 1110067B02Rik | 909 | 963 |
| NM_026614 | 2900002J19Rik | 910 | 964 |
| AK008267 | 1810055D05Rik | 911 | 965 |
| AK009374 | 2310016A09Rik | 912 | 966 |
| AK003283 | Mrpl13 | 913 | 967 |
| NM_011058 | Pdgfra | 914 | 968 |
| AK002593 | Cox7b | 915 | 969 |
| AK005080 | Suclg1 | 916 | 970 |
| AK002889 | 0610041L09Rik | 917 | 971 |
| BC005585 | LOC231086 | 918 | 972 |
| NM_020520 | Slc25a20 | 919 | 973 |
| AK002320 | 0610008C08Rik | 920 | 974 |
| BG172638 | LOC218885 | 921 | 975 |
| BC005792 | Pte1 | 922 | 976 |
| AK003975 | 1500004O06Rik | 923 | 977 |
| | | | 978 |
| NM_021532 | Thyex3-pending | 924 | 979 |
| AK009364 | 1810015H18Rik | 925 | 980 |
| AK002452 | 1110008F13Rik | 926 | 981 |
| BC004020 | BC004020 | 927 | 982 |
| BB004706 | MGC37634 | 928 | 983 |
| NM_013898 | Timm8a | 929 | 984 |
| AK004827 | 0610011D08Rik | 930 | 985 |
| AK004924 | Nudt7 | 931 | 986 |
| AK003393 | Idh3a | 932 | 987 |
| AJ250489 | Ramp1 | 933 | 988 |
| X01756 | Cycs | 934 | 989 |
| BC009134 | AA959601 | 935 | 990 |
| AI648018 | 2610207I16Rik | 936 | 991 |
| | | | 992 |
| | | | 993 |
| AJ131522 | Mlycd | 937 | 994 |
| AF278699 | Angpt14 | 938 | 995 |
| | | | 996 |
| | | | 997 |
| NM_013743 | Pdk4 | 42 | 93 |
| | | | 94 |
| | | | 998 |
| Z71189 | Acadvl | 939 | 999 |
| | | | 1000 |
| | | | 1001 |
| AF030343 | Ech1 | 940 | 1002 |
| D13664 | Osf2-pending | 941 | 1003 |
| D50834 | Cyp4bl | 942 | 1004 |
| L12447 | Igfbp5 | 45 | 97 |
| M93275 | Adfp | 943 | 1005 |
| | | | 1006 |
| | | | 1007 |
| M96163 | Snk | 944 | 1008 |
| U07159 | Acadm | 945 | 1009 |
| | | | 1010 |
| | | | 1011 |
| U21489 | Acadl | 946 | 1012 |
| | | | 1013 |
| | | | 1014 |
| U37501 | Lama5 | 947 | 1015 |
| X70398 | D0H4S114 | 948 | 1016 |
| X89998 | Hsd17b4 | 949 | 1017 |
| | | | 1018 |
| | | | 1019 |

Toxicity values were calculated from the expression pattern of the 74 probes (SEQ ID NOs: 950-1019, 863, 93, 94, 97) of the toxicity-related population of genes in the following manner. The gene expression profile induced by rosiglitazone (used at an effective concentration of 600 nM) was used as template, and a scale factor S of a given treatment was determined to minimize the following X²:

$${χ}^{2} = {\sum }_{i = 1}^{74} {( S * {R}_{1} - {X}_{1} )}^{2} / ( {σ}_{Ri}^{2} + {σ}_{Xi}^{2} )$$

where Ri stands for the log(ratio) of the 74 probes whose expression was affected by the high dose of rosiglitazone, σ_{Ri} is the error of Ri, Xi stands for the log(ratio) of the 74 probes (SEQ ID NOs: 950-1019, 863, 93, 94, 97) from that treatment, and σ_{Xi} is the error of Xi. The scale factor S is defined as the toxicity value for that treatment.

To determine whether the toxicity values, calculated in the foregoing manner, correlated with an increase in heart weight in vivo, heart weights were plotted directly against the calculated toxicity values for 10 full or partial agonists of PPARγ that were tested both in vivo in rat, and in vitro in 3T3L1 cell lines. The data used was obtained from administration of the highest dosage of each of the 10 compounds. The calculated toxicity values for 9 of the 10 compounds correlated highly with the in vivo heart weights (correlation 0.8, P-value=1.8×10⁻³). The fact that the calculated toxicity value for one of the 10 compounds did not correlate highly with the in vivo heart weight was probably because the dosage of this compound, in vivo, was relatively low (30 milligrams per kilogram body weight) compared to the dosage of the other nine compounds (>100 milligrams per kilogram body weight).

Thus, the 3T3L1 cell line is useful in the practice of the present invention to obtain gene expression data that correlates with an undesirable increase in heart weight caused by a PPARγ agonist or antagonist.

Early Heart Weight Biomarkers in EWAT: EWAT responded to treatment with a PPARγ agonist, or partial agonist, much more strongly than heart tissues. Therefore EWAT was a sensitive tissue in terms of magnitude of response. The 355 probes (SEQ ID NOs: 551-894, 155, 157, 164, 171, 178, 179, 185, 188, 204-206) corresponding to the toxicity-related population of 343 genes (SEQ ID NOs: 219-550, 104, 105, 112, 119, 126, 127, 133, 136, 149-151), described in this Example, were further analyzed to identify a sub-population of genes that are useful as early biomarkers for the onset of the adverse effect of heart weight increase due to administration of a PPARγ agonist or partial agonist.

The 355 rat EWAT probes (SEQ ID NOs: 551-894, 155, 157, 164, 171, 178, 179, 185, 188, 204-206) were projected to the “747 tissue experiment” by homolog mapping, and then selecting the subset of PPARγ regulated genes from fat tissues. 46 mouse homologs were regulated in the one day and 2 day treatments. These 46 genes are useful in the practice of the present invention as a toxicity-related gene population. The nucleotide sequences of the 67 probes that hybridized to the 46 genes, identified in Table 8, (SEQ ID NOs: 1036-1057, 951, 955, 957, 863, 959, 960, 63, 962, 966, 971-974, 980, 981, 984, 987, 989, 991-996, 93, 94, 998-1001, 97, 1004-1014, 1017-1019), are set forth in the SEQUENCE LISTING. The nucleotide sequences of the corresponding 46 genes identified in Table 8, (SEQ ID NOs: 1020-1035, 896, 900, 902, 903, 905, 906, 13, 908, 912, 917-920, 925, 926, 929, 932, 934, 936-939, 42, 942-946, 45, 949), are set forth in the SEQUENCE LISTING. Among the 46 genes (SEQ ID NOs: 1020-1035, 896, 900, 902, 903, 905, 906, 13, 908, 912, 917-920, 925, 926, 929, 932, 934, 936-939, 42, 942-946, 45, 949) regulated in the mouse fat tissues, 44 probes overlapped with the 74 3T3L1 probes (SEQ ID NOs: 950-1019, 863, 93, 94, 97).

TABLE 8
PPARγ_Mouse_eWAT_Toxicity_HeartWeight_EarlyProbe_67
(Species: Mouse)

| Accession | | Gene SEQ | Probe SEQ |
|---|---|---|---|
| number | Gene Name | ID NO | ID NO |
| AK010479 | 2410012P20Rik | 1020 | 1036 |
| AK013511 | Ndufv2 | 896 | 951 |
| NM_026179 | 1300003D03Rik | 900 | 955 |
| NM_008303 | Hspe1 | 1021 | 1037 |
| NM_025384 | 1110003P16Rik | 902 | 957 |
| AK008511 | Usmg5 | 903 | 863 |
| NM_011192 | Psme3 | 1022 | 1038 |
| BC004045 | LOC212442 | 905 | 959 |
| AK018125 | Gfm | 1023 | 1039 |
| AK005067 | Chp-pending | 906 | 960 |
| AK004867 | 1300002P22Rik | 13 | 63 |
| AF058955 | Sucla2 | 1024 | 1040 |
| AK002483 | 0610010I20Rik | 908 | 962 |
| NM_019975 | Hpcl-pending | 1025 | 1041 |
| AK009575 | Bdh | 1026 | 1042 |
| AK008788 | 2610003B19Rik | 1027 | 1043 |
| AK009374 | 2310016A09Rik | 912 | 966 |
| AK013955 | 3110001K13Rik | 1028 | 1044 |
| AK003325 | 1110002N22Rik | 1029 | 1045 |
| AK002889 | 0610041L09Rik | 917 | 971 |
| BC005585 | LOC231086 | 918 | 972 |
| NM_020520 | Slc25a20 | 919 | 973 |
| NM_019961 | Pex3 | 1030 | 1046 |
| NM_026494 | AI413471 | 1031 | 1047 |
| AK002320 | 0610008C08Rik | 920 | 974 |
| AK009364 | 1810015H18Rik | 925 | 980 |
| AK002452 | 1110008F13Rik | 926 | 981 |
| NM_013898 | Timm8a | 929 | 984 |
| AK015530 | 4930469P12Rik | 1032 | 1048 |
| AK003393 | Idh3a | 932 | 987 |
| AI195543 | MGC29978 | 1033 | 1049 |
| X01756 | Cycs | 934 | 989 |
| AI648018 | 2610207I16Rik | 936 | 991 |
| | | | 992 |
| | | | 993 |
| Z14050 | Dci | 1034 | 1050 |
| AJ131522 | Mlycd | 937 | 994 |
| | | | 1051 |
| AF278699 | Angptl4 | 938 | 995 |
| | | | 996 |
| NM_013743 | Pdk4 | 42 | 93 |
| | | | 998 |
| | | | 94 |
| Z71189 | Acadvl | 939 | 999 |
| | | | 1000 |
| | | | 1001 |
| D50834 | Cyp4b1 | 942 | 1052 |
| | | | 1053 |
| | | | 1004 |
| L12447 | Igfbp5 | 45 | 1054 |
| | | | 97 |
| | | | 1055 |
| M93275 | Adfp | 943 | 1005 |
| | | | 1006 |
| | | | 1007 |
| M96163 | Snk | 944 | 1008 |
| U01163 | Cpt2 | 1035 | 1056 |
| | | | 1057 |
| U07159 | Acadm | 945 | 1011 |
| | | | 1010 |
| | | | 1009 |
| U21489 | Acadl | 946 | 1012 |
| | | | 1013 |
| | | | 1014 |
| X89998 | Hsd17b4 | 949 | 1018 |
| | | | 1017 |
| | | | 1019 |

Plasma Volume Expansion Biomarkers in EWAT and 3T3L1 Cells: Using the same procedure that is described in this Example in the section entitled “Measuring the Toxic Effects of PPARγ Agonists and PPARγ Partial Agonists in Rats” for identifying heart weight biomarkers in EWAT, 271 probes were identified in EWAT whose expression was affected by a PPARγ full agonist or partial agonist, and that correlated with plasma volume expansion (PVE). The nucleotide sequences of the 271 probes identified in Table 9, (SEQ ID NOs: 1239-1428, 558, 561, 158, 565, 574, 576, 578, 585, 592, 597, 600, 609, 612, 613, 617, 163, 625, 641-643, 646, 647, 655-657, 661, 666, 171, 681, 697, 700, 706, 707, 712, 720, 727, 740, 745, 748, 749, 755-757, 762, 766, 767, 769-771, 773, 778, 780, 786, 789, 794, 800, 803, 804, 188, 189, 191, 813, 814, 822, 823, 556, 828, 831, 832, 836, 840, 844, 864, 871, 876, 878, 883, 884, 889-891), are set forth in the SEQUENCE LISTING. 259 genes correspond to the 271 probes (SEQ ID NOs: 1239-1428, 558, 561, 158, 565, 574, 576, 578, 585, 592, 597, 600, 609, 612, 613, 617, 163, 625, 641-643, 646, 647, 655-657, 661, 666, 171, 681, 697, 700, 706, 707, 712, 720, 727, 740, 745, 748, 749, 755-757, 762, 766, 767, 769-771, 773, 778, 780, 786, 789, 794, 800, 803, 804, 188, 189, 191, 813, 814, 822, 823, 556, 828, 831, 832, 836, 840, 844, 864, 871, 876, 878, 883, 884, 889-891). The nucleotide sequences of these 259 genes as identified in Table 9 (SEQ ID NOs: 1058-1238, 222, 224, 106, 226, 235, 237, 239, 246, 253, 258, 261, 270, 273, 274, 278, 111, 286, 302-304, 307, 308, 316-318, 322, 327, 119, 342, 358, 361, 367, 368, 373, 381, 388, 401, 406, 409, 410, 416-418, 423, 427, 428, 430-432, 434, 439, 441, 447, 450, 455, 461, 464, 465, 136, 137, 139, 474, 475, 482, 485, 488, 491, 492, 496, 500, 504, 524, 530, 534, 536, 541, 542, 547), are set forth in the SEQUENCE LISTING.

TABLE 9
PPARγ_Rat_eWAT_Toxicity_{—}
PVE_Probe_271 (Species: Rat)

| Accession | | Gene SEQ | Probe |
|---|---|---|---|
| number | Gene Name | ID NO | SEQ ID NO |
| J02752 | RATACOA1 | 1058 | 1239 |
| | | | 1240 |
| J05030 | Acads | 1059 | 1241 |
| | | | 1242 |
| K03249 | Ehhadh | 222 | 558 |
| M17701 | Gapd | 1060 | 1243 |
| | | | 1244 |
| | | | 1245 |
| M29853 | Cyp4b1 | 224 | 561 |
| AA875107 | AA875107 | 1061 | 1246 |
| U39208 | CYP4F6 | 1062 | 1247 |
| U68544 | cyclophilin D | 1063 | 1248 |
| Y09333 | Mte1 | 106 | 158 |
| AI170251 | g3710291 | 226 | 565 |
| AW523642 | g4133650 | 1064 | 1249 |
| 701221122H1 | 701221122H1 | 1065 | 1250 |
| BF288270 | g2937947 | 1066 | 1251 |
| BF415385 | g3711895 | 1067 | 1252 |
| G3332690 | g3332690 | 1068 | 1253 |
| G3705868 | g3705868 | 1069 | 1254 |
| BE111773 | g2938661 | 1070 | 1255 |
| G3708088 | g3708088 | 1071 | 1256 |
| G2936894 | g2936894 | 1072 | 1257 |
| AW918940 | g4134740 | 1073 | 1258 |
| AI113016 | g3512965 | 235 | 574 |
| G3103828 | g3103828 | 237 | 576 |
| G3816318 | g3816318 | 1074 | 1259 |
| AI408705 | g2863227 | 239 | 578 |
| G3710568 | g3710568 | 1075 | 1260 |
| G979671 | g979671 | 1076 | 1261 |
| BF420654 | g3227012 | 1077 | 1262 |
| G3189034 | g3189034 | 246 | 585 |
| G2948676 | g2948676 | 1078 | 1263 |
| G2939411 | g2939411 | 1079 | 1264 |
| AI144876 | Ass | 253 | 592 |
| G2948912 | g2948912 | 1080 | 1265 |
| AI411031 | g3709121 | 258 | 597 |
| G2862965 | g2862965 | 261 | 600 |
| G4132595 | g4132595 | 1081 | 1266 |
| G3812213 | g3812213 | 1082 | 1267 |
| BG373361 | g3333793 | 1083 | 1268 |
| G2672793 | g2672793 | 1084 | 1269 |
| G3292487 | g3292487 | 1085 | 1270 |
| G3226140 | g3226140 | 1086 | 1271 |
| G3727666 | g3727666 | 1087 | 1272 |
| G3730290 | g3730290 | 1088 | 1273 |
| BE109153 | g3638407 | 1089 | 1274 |
| BF560807 | g3187199 | 270 | 609 |
| G3071873 | g3071873 | 273 | 612 |
| AA799476 | g2862431 | 274 | 613 |
| G3708991 | g3708991 | 1090 | 1275 |
| AI411212 | g3710380 | 278 | 617 |
| BG376920 | g2864026 | 1091 | 1276 |
| G3187055 | g3187055 | 1092 | 1277 |
| 701221494H1 | 701221494H1 | 1093 | 1278 |
| G3396562 | g3396562 | 1094 | 1279 |
| AI138016 | g3638793 | 1095 | 1280 |
| G3709353 | g3709353 | 1096 | 1281 |
| G3816414 | g3816414 | 1097 | 1282 |
| AA848702 | g2936242 | 1098 | 1283 |
| G3638603 | g3638603 | 111 | 163 |
| G3813131 | g3813131 | 286 | 625 |
| G3102919 | g3102919 | 1099 | 1284 |
| AI013919 | g4133944 | 1100 | 1285 |
| AI104605 | g4134272 | 1101 | 1286 |
| BG378613 | g3103045 | 1102 | 1287 |
| BG381472 | g3726883 | 1103 | 1288 |
| G2979890 | g2979890 | 1104 | 1289 |
| G2937670 | g2937670 | 1105 | 1290 |
| AA850195 | g2937735 | 1106 | 1291 |
| g3706559 | g3706559 | 1107 | 1292 |
| AA800179 | g2863134 | 1108 | 1293 |
| AI230578 | g3814465 | 1109 | 1294 |
| BE109153 | g3637263 | 1110 | 1295 |
| g3636884 | g3636884 | 302 | 641 |
| AA848951 | g2936491 | 1111 | 1296 |
| BF284475 | g3711260 | 303 | 642 |
| AA799707 | g4131430 | 1112 | 1297 |
| AA894090 | g3020969 | 304 | 643 |
| BE113034 | g3815452 | 307 | 646 |
| G3397918 | g3397918 | 1113 | 1298 |
| G3828291 | g3828291 | 1114 | 1299 |
| G3137782 | g3137782 | 308 | 647 |
| G3728910 | g3728910 | 1115 | 1300 |
| AI229639 | g3813526 | 1116 | 1301 |
| AI170808 | g3710848 | 316 | 655 |
| AA963282 | g3136774 | 1117 | 1302 |
| G3727129 | g3727129 | 317 | 656 |
| AW528443 | g4136134 | 318 | 657 |
| G3333614 | g3333614 | 1118 | 1303 |
| BE110615 | g3226627 | 1119 | 1304 |
| G3512087 | g3512087 | 1120 | 1305 |
| BF556962 | g3708808 | 322 | 661 |
| G3712131 | g3712131 | 1121 | 1306 |
| AW916776 | g3667631 | 1122 | 1307 |
| G2889306 | g2889306 | 1123 | 1308 |
| G3398898 | g3398898 | 1124 | 1309 |
| AA963069 | g3136561 | 327 | 666 |
| AI071994 | g3398188 | 1125 | 1310 |
| AA858867 | g2948218 | 1126 | 1311 |
| AI170067 | g3710107 | 119 | 171 |
| AI412011 | g3247895 | 1127 | 1312 |
| g3511496 | g3511496 | 1128 | 1313 |
| G3710033 | g3710033 | 1129 | 1314 |
| BE109401 | g3247351 | 1130 | 1315 |
| G3019865 | g3019865 | 1131 | 1316 |
| G3813191 | g3813191 | 1132 | 1317 |
| G3815059 | g3815059 | 1133 | 1318 |
| G4132386 | g4132386 | 1134 | 1319 |
| g3398472 | g3398472 | 1135 | 1320 |
| AA819658 | g2888922 | 1136 | 1321 |
| AA998205 | g3188856 | 342 | 681 |
| AA924580 | g3071716 | 1137 | 1322 |
| G980031 | g980031 | 1138 | 1323 |
| 700691760H1 | 700691760H1 | 1139 | 1324 |
| AI234620 | g3828126 | 1140 | 1325 |
| 701216507H1 | 701216507H1 | 358 | 697 |
| BG380734 | g2938750 | 1141 | 1326 |
| BG377008 | g2863410 | 361 | 700 |
| AW918113 | g3291307 | 1142 | 1327 |
| G3730272 | g3730272 | 1143 | 1328 |
| AI058968 | g3332745 | 367 | 706 |
| 701349156H1 | 701349156H1 | 368 | 707 |
| 700692031H1 | 700692031H1 | 1144 | 1329 |
| G980946 | g980946 | 1145 | 1330 |
| 701219843H1 | 701219843H1 | 1146 | 1331 |
| AI577393 | g980620 | 1147 | 1332 |
| 701350827H1 | 701350827H1 | 1148 | 1333 |
| 700506509H1 | 700506509H1 | 1149 | 1334 |
| 700508607H1 | 700508607H1 | 373 | 712 |
| 600512417R1 | 600512417R1 | 381 | 720 |
| G4134738 | g4134738 | 388 | 727 |
| 600521579R1 | 600521579R1 | 1150 | 1335 |
| 600519254R1 | 600519254R1 | 1151 | 1336 |
| G3225638 | g3225638 | 401 | 740 |
| 600518885R1 | 600518885R1 | 1152 | 1337 |
| 600524228R1 | 600524228R1 | 1153 | 1338 |
| AI010433 | Cdtw 1 | 406 | 745 |
| G3710810 | g3710810 | 1154 | 1339 |
| BG381033 | g4131620 | 409 | 748 |
| 600512426R1 | 600512426R1 | 410 | 749 |
| AW915824 | 600510363R1 | 1155 | 1340 |
| 600518233R1 | 600518233R1 | 1156 | 1341 |
| AI599296 | g3711488 | 1157 | 1342 |
| G3103745 | g3103745 | 1158 | 1343 |
| G4134262 | g4134262 | 416 | 755 |
| AI009817 | g3223649 | 1159 | 1344 |
| 600523104R1 | 600523104R1 | 417 | 756 |
| 600520906R1 | 600520906R1 | 418 | 757 |
| AI101492 | g4134011 | 1160 | 1345 |
| AA892500 | g3019379 | 1161 | 1346 |
| AI411374 | g3709749 | 1162 | 1347 |
| G3815486 | g3815486 | 423 | 762 |
| 600512215R1 | 600512215R1 | 1163 | 1348 |
| BG376528 | g3707272 | 1164 | 1349 |
| 600519560R1 | 600519560R1 | 1165 | 1350 |
| AA800476 | g2863431 | 1166 | 1351 |
| G3104296 | g3104296 | 427 | 766 |
| 600514084R1 | 600514084R1 | 428 | 767 |
| BF394796 | 600515077R1 | 1167 | 1352 |
| 600508574R1 | 600508574R1 | 430 | 769 |
| 600516676R1 | 600516676R1 | 1168 | 1353 |
| G3036598 | g3036598 | 1169 | 1354 |
| AA875107 | g2980055 | 431 | 770 |
| AI104528 | g3708870 | 432 | 771 |
| AA799741 | g2862696 | 1170 | 1355 |
| AJ005161 | EF-Ts | 1171 | 1356 |
| G3104097 | g3104097 | 1172 | 1357 |
| AI171656 | g3711696 | 434 | 773 |
| 700506775H1 | 700506775H1 | 1173 | 1358 |
| AI104348 | g3708719 | 439 | 778 |
| AI045456 | g3292275 | 1174 | 1359 |
| G3831232 | g3831232 | 441 | 780 |
| BE349717 | g3020180 | 1175 | 1360 |
| G976906 | g976906 | 1176 | 1361 |
| BE101298 | g3334069 | 1177 | 1362 |
| G3019879 | g3019879 | 447 | 786 |
| g3018118 | g3018118 | 1178 | 1363 |
| BG381624 | g3018621 | 450 | 789 |
| 700688496H1 | 700688496H1 | 1179 | 1364 |
| AI145756 | g3667555 | 1180 | 1365 |
| BF282282 | g3730624 | 1181 | 1366 |
| AA801227 | g4131587 | 1182 | 1367 |
| AA800782 | g4131537 | 455 | 794 |
| BF413204 | g3726768 | 1183 | 1368 |
| AI071674 | g3397889 | 1184 | 1369 |
| AA859467 | g2948987 | 1185 | 1370 |
| G4135910 | g4135910 | 461 | 800 |
| BF282978 | g3019668 | 1186 | 1371 |
| BF394796 | g3332553 | 1187 | 1372 |
| G978793 | g978793 | 464 | 803 |
| G3707669 | g3707669 | 465 | 804 |
| G3709693 | g3709693 | 1188 | 1373 |
| AI231798 | g3815678 | 1189 | 1374 |
| AI227820 | Mgll | 136 | 188 |
| G3813792 | g3813792 | 1190 | 1375 |
| g3104887 | g3104887 | 1191 | 1376 |
| AA892864 | Mgll | 137 | 189 |
| G3222645 | g3222645 | 1192 | 1377 |
| G977669 | g977669 | 139 | 191 |
| AW253370 | g3104091 | 474 | 813 |
| AA965106 | g3138598 | 475 | 814 |
| G3812897 | g3812897 | 1193 | 1378 |
| AW913838 | g3222273 | 1194 | 1379 |
| D10952 | Cox5b | 1195 | 1380 |
| J02749 | Acaa | 482 | 822 |
| | | | 823 |
| L11276 | Acadl | 485 | 556 |
| D16236 | Cdc25a | 1196 | 1381 |
| NM_012891 | Acadvl | 488 | 828 |
| AF061266 | Trrp1 | 1197 | 1382 |
| X68101 | trg | 491 | 831 |
| NM_022398 | LOC64201 | 492 | 832 |
| NM_022182 | Fgf7 | 1198 | 1383 |
| NM_013168 | Hmbs | 1199 | 1384 |
| AF139830 | Igfbp-5 | 496 | 836 |
| AB028626 | Rasa3 | 500 | 840 |
| M29341 | Gapd | 1200 | 1243 |
| | | | 1385 |
| AW917188 | Dpyd | 1201 | 1386 |
| | | | 1387 |
| AF044574 | Decr2 | 1202 | 1388 |
| M96374 | Nrxn1 | 1203 | 1389 |
| AF170918 | Aldh9a1 | 1204 | 1390 |
| | | | 1391 |
| NM_031032 | Gmfb | 504 | 844 |
| NM_017280 | Psma3 | 1205 | 1392 |
| NM_012569 | Gls | 1206 | 1393 |
| AB052846 | Sc5d | 1207 | 1394 |
| NM_017020 | Il6r | 1208 | 1395 |
| NM_021767 | Nrxn1 | 1209 | 1396 |
| L35921 | Gng8 | 1210 | 1397 |
| NM_017183 | Il8rb | 1211 | 1398 |
| AB006614 | Ucp3 | 1212 | 1399 |
| | | | 1400 |
| | | | 1401 |
| NM_023023 | Crmp5 | 1213 | 1402 |
| NM_017321 | Ratireb | 1214 | 1403 |
| AF150091 | Timm10 | 1215 | 1404 |
| NM_019352 | Timm23 | 1216 | 1405 |
| AF019109 | Sort1 | 1217 | 1406 |
| NM_031062 | Mvd | 1218 | 1407 |
| AF026554 | Slc5a6 | 1219 | 1408 |
| J05446 | Gys2 | 1220 | 1409 |
| NM_022541 | Ddp2 | 1221 | 1410 |
| NM_031151 | Mor1 | 1222 | 1411 |
| AF021854 | Pecr | 1223 | 1412 |
| NM_017256 | Tgfbr3 | 1224 | 1413 |
| NM_024398 | Aco2 | 1225 | 1414 |
| NM_023964 | Gapds | 1226 | 1415 |
| D28560 | Enpp2 | 1227 | 1416 |
| AF150082 | Timm8a | 524 | 864 |
| NM_031527 | Ppp1ca | 1228 | 1417 |
| X54510 | Atp5j | 1229 | 1418 |
| NM_024148 | Apex | 1230 | 1419 |
| X05634 | Sod1 | 530 | 871 |
| NM_022500 | Ftl1 | 1231 | 1420 |
| NM_017006 | G6pd | 1232 | 1421 |
| NM_024001 | RPT | 534 | 876 |
| X97831 | Slc25a20 | 536 | 878 |
| D88891 | Bach | 1233 | 1422 |
| AB041723 | Pdcd8 | 541 | 883 |
| AF285103 | Psmb7 | 542 | 884 |
| AY034383 | Dlc2 | 1234 | 1423 |
| U88295 | Cpt2 | 547 | 889 |
| | | | 890 |
| | | | 891 |
| NM_017177 | Chetk | 1235 | 1424 |
| U00926 | Atp5d | 1236 | 1425 |
| J04044 | Alas1 | 1237 | 1426 |
| | | | 1427 |
| AF239045 | Kidins220 | 1238 | 1428 |

Mapping these 271 EWAT probes (SEQ ID NOs: 1239-1428, 558, 561, 158, 565, 574, 576, 578, 585, 592, 597, 600, 609, 612, 613, 617, 163, 625, 641-643, 646, 647, 655-657, 661, 666, 171, 681, 697, 700, 706, 707, 712, 720, 727, 740, 745, 748, 749, 755-757, 762, 766, 767, 769-771, 773, 778, 780, 786, 789, 794, 800, 803, 804, 188, 189, 191, 813, 814, 822, 823, 556, 828, 831, 832, 836, 840, 844, 864, 871, 876, 878, 883, 884, 889-891) to mice yielded 44 probes that were also regulated by PPARγ agonists in the mouse 3T3L1 cell line. The nucleotide sequences of the 44 probes identified in Table 10, (SEQ ID NOs: 1449-1471, 952, 956, 957, 963, 975, 976, 981, 983, 984, 986, 990, 999-1001, 1004-1007, 1012-1014), are set forth in the SEQUENCE LISTING. The nucleotide sequences of the corresponding 35 genes identified in Table 10, (SEQ ID NOs: 1429-1448, 897, 901, 902, 919, 921, 922, 926, 928, 929, 931, 935, 939, 942, 943, 946), are set forth in the SEQUENCE LISTING.

TABLE 10
PPARγ_3T3L1_Toxicity_{—}
PVE_Probe_44 (Species: Mouse Cell Line)

| Accession | | Gene SEQ | Probe |
|---|---|---|---|
| number | Gene Name | ID NO | SEQ ID NO |
| BC004645 | Aco2 | 1429 | 1449 |
| | | | 1450 |
| AK004125 | 1110036H20Rik | 897 | 952 |
| AK007415 | 1810010A06Rik | 901 | 956 |
| AK007651 | Ubqln1 | 1430 | 1451 |
| NM_025384 | 1110003P16Rik | 902 | 957 |
| NM_015744 | Enpp2 | 1431 | 1452 |
| NM_019993 | Aldh9a1 | 1432 | 1453 |
| BC011289 | 6720463E02Rik | 1433 | 1454 |
| AK004193 | 1110046O21Rik | 1434 | 1455 |
| AK004954 | 1300010A20Rik | 1435 | 1456 |
| AK007497 | 1810014L12Rik | 1436 | 1457 |
| NM_024207 | 1110021N07Rik | 1437 | 1458 |
| AK004634 | Gng31g | 1438 | 1459 |
| AK008088 | Timm13a | 1439 | 1460 |
| NM_020520 | Slc25a20 | 919 | 973 |
| AJ309922 | Mvd | 1440 | 1461 |
| BG172638 | LOC218885 | 921 | 975 |
| BC005792 | Pte1 | 922 | 976 |
| NM_016897 | Timm23 | 1441 | 1462 |
| AK002452 | 1110008F13Rik | 926 | 981 |
| BC002251 | AI480570 | 1442 | 1463 |
| BB004706 | MGC37634 | 928 | 983 |
| NM_007658 | Cdc25a | 1443 | 1464 |
| NM_013898 | Timm8a | 929 | 984 |
| AK004924 | Nudt7 | 931 | 986 |
| BC009134 | AA959601 | 935 | 990 |
| Z71189 | Acadvl | 939 | 999 |
| | | | 1000 |
| | | | 1001 |
| AF006688 | Acox1 | 1444 | 1465 |
| | | | 1466 |
| | | | 1467 |
| D50834 | Cyp4b1 | 942 | 1004 |
| M16229 | Mor1 | 1445 | 1468 |
| M93275 | Adfp | 943 | 1005 |
| | | | 1006 |
| | | | 1007 |
| U21489 | Acadl | 946 | 1012 |
| | | | 1013 |
| | | | 1014 |
| X53802 | Il6ra | 1446 | 1469 |
| AB016248 | Sc5d | 1447 | 1470 |
| NM_008008 | Fgf7 | 1448 | 1471 |

It is noteworthy that the heart weight and PVE toxicity values from the 3T3L1 model system were highly correlated with the classifier values as described in Example 3. Therefore, in this example, using the 3T3L1 system, only the toxicity value or the classifier need be calculated for each compound.

### EXAMPLE 3

This Example describes the identification of a classifier population of genes that is useful for classifying candidate agents as being more like a known agonist of PPARγ, or as being more like a known partial agonist of PPARγ.

The gene expression profile of 26 compounds at high dosage (30×EC₅₀) in 3T3L1 adipocyte cell line were measured using a Rosetta mouse 25K DNA Microarray. The overall experiment was conducted in three phases (i.e., in three separate experiments conducted at three different times) as shown in Table 11 below. Three replicates were done for each of the tested compounds in each phase of the experiment.

The gene expression measurement levels from the following compound treatments were used as the training set: PPARγ partial agonists: 2-(3-{[3-(4-chlorobenzoyl)-2-methyl-6-(trifluoromethoxy)-1H-indol-1-yl]methyl} phenoxy)-3-methylbutanoate; (2R)-2-(4-chloro-3-{[3-(6-methoxy-1,2-benzisoxazol-3-yl)-2-methyl-6-(trifluoromethoxy)-1H-indol-1-yl]methyl}phenoxy)propanoate; (2S)-2-(4-chloro-3-{[1-(6-chloro-1,2-benzisoxazol-3-yl)-2-methyl-5-(trifluoromethoxy)-1H-indol-3-yl]oxy}phenoxy)propanoic acid; and (2R)-2-(2-chloro-5-{[3-(4-chlorobenzoyl)-2-methyl-6-(trifluoromethoxy)-1H-indol-1-yl]methyl} phenoxy)propanoic acid; and PPARγ agonists: 5-(4-{2-[methyl(pyridin-2-yl)amino]ethoxy} benzyl)-1,3-thiazolidine-2,4-dione, and 5-{4-[2-hydroxy-2-(5-methyl-2-phenyl-1,3-oxazol-4-yl)ethoxy]benzyl}-1,3-thiazolidine-2,4-dione.

The other PPARγ agonist, and partial agonist, compounds were used in testing the classifier population of genes. The following dosages were used where indicated by a * 0.540 μM in Phase 1, 0.600 μM in Phases 2 and 3; and where indicated by a ** 6.3 μM in Phase 2, 6.324 μM in Phase 3. The PPARα agonist was included as a control.

TABLE 11

| Phase | Phase | Phase | | Dosage |
|---|---|---|---|---|
| 1 | 2 | 3 | Compounds | (μM) |
| | X | X | PPARα agonist | 10.0 |
| | X | | Partial agonist 2 | 0.030 |
| X | | | Partial agonist 3 | 0.300 |
| | X | X | Partial agonist 4 | ** |
| | X | | Partial agonist 2-(3-{[3-(4- | 3.0 |
| | | | chlorobenzoyl)-2-methyl-6- | |
| | | | (trifluoromethoxy)-1H-indol-1- | |
| | | | yl]methyl}phenoxy)-3- | |
| | | | methylbutanoate | |
| X | X | X | Partial agonist (2R)-2-(4-chloro-3- | * |
| | | | {[3-(6-methoxy-1,2- | |
| | | | benzisoxazol-3-yl)-2-methyl-6- | |
| | | | (trifluoromethoxy)-1H-indol-1- | |
| | | | yl]methyl}phenoxy)propanoate | |
| | X | | Partial agonist 5 | 0.3 |
| | X | | Partial agonist 6 | 10.0 |
| X | | | Partial agonist (2S)-2-(4-chloro-3- | 0.12 |
| | | | {[1-(6-chloro- 1,2- | |
| | | | benzisoxazol-3-yl)-2-methyl-5- | |
| | | | (trifluoromethoxy)-1H-indol-3- | |
| | | | yl]oxy}phenoxy)propanoic acid | |
| | X | | Partial agonist 7 | 1.4 |
| | X | | Partial agonist 8 | 0.1 |
| | | X | Partial agonist 9 | 0.158 |
| | | X | Partial agonist 10 | 0.285 |
| X | | | Partial agonist (2R)-2-(2-chloro-5- | 0.054 |
| | | | {[3-(4- chlorobenzoyl)-2- | |
| | | | methyl-6-(trifluoromethoxy)-1H- | |
| | | | indol-1-yl]methyl}phenoxy)pro- | |
| | | | panoic acid | |
| | X | X | Partial agonist 11 | 1.1 |
| | | X | Partial agonist 12 | 0.221 |
| | X | X | Partial agonist 13 | 1.8 |
| | | X | Partial agonist 14 | 0.126 |
| | | X | Partial agonist 15 | 0.2 |
| | | X | Partial agonist 16 | 16.032 |
| | | X | Partial agonist 17 | 1.075 |
| X | | X | Agonist 1 | 3.870 |
| X | | | Agonist 2 | 0.006 |
| | X | | Agonist 3 | 1.5 |
| X | X | X | Agonist 5-(4-{2-[methyl(pyridin- | * |
| | | | 2-yl)amino]ethoxy}benzyl)-1,3- | |
| | | | thiazolidine-2,4-dione) | |
| X | | | Agonist (5-{4-[2-hydroxy-2-(5- | 0.027 |
| | | | methyl-2-phenyl-1,3- oxazol-4- | |
| | | | yl)ethoxy]benzyl}-1,3- | |
| | | | thiazolidine-2,4-dione) | |

The three replicate gene expression profiles within each phase of the experiment were first combined based on the error-weighted average. Expression profiles of two PPARγ full agonists, and four PPARγ partial agonists (in Phase 1) were chosen for classifier training, and were divided into the following two groups:

Group 1: two PPARγ full agonists (5-(4-{2-[methyl(pyridin-2-yl)amino]ethoxy} benzyl)-1,3-thiazolidine-2,4-dione and 5-{4-[2-hydroxy-2-(5-methyl-2-phenyl-1,3-oxazol-4-yl)ethoxy]benzyl}-1,3-thiazolidine-2,4-dione)

Group 2: four PPARγ partial agonists ((2R)-2-(2-chloro-5-{[3-(4-chlorobenzoyl)-2-methyl-6-(trifluoromethoxy)-1H-indol-1-yl]methyl}phenoxy)propanoic acid; (2S)-2-(4-chloro-3-{[1-(6-chloro-1,2-benzisoxazol-3-yl)-2-methyl-5-(trifluoromethoxy)-1H-indol-3-yl]oxy}phenoxy)propanoic acid; (2S)-2-(3-{[1-(4-methoxybenzoyl)-2-methyl-5-(trifluoromethoxy)-1H-indol-3-yl]methyl}phenoxy)propanoic acid; and (2R)-2-(4-chloro-3-{[3-(6-methoxy-1,2-benzisoxazol-3-yl)-2-methyl-6-(trifluoromethoxy)-1H-indol-1-yl]methyl} phenoxy)propanoate).

The expression profiles of the remaining compounds were used to test the classifier gene population.

Probes identified in the training gene set that had a pvalue of less than 0.1 in at least one of the above training compound expression profiles were selected. A total of 7,610 probes were selected. The Matlab function ANOVA1 (one-way analysis of variance) was used to calculate the pvalue (hereafter referred to as the ANOVA-pvalue) for the null hypothesis that the means of Group 1 and Group 2 are equal. Probes with an ANOVA-pvalue smaller than 1×10⁻⁷ and an absolute value of the average of logRatio in Group 1 greater than log₁₀ 1.5 (which is a value of 0.1761) were selected. The resulting 303 probes corresponded to 290 genes that were the classifier population that were PPARγ agonist signature genes and that best distinguished partial PPARγ agonists from full PPARγ agonists.

The nucleotide sequences of the 303 probes identified in Table 12, (SEQ ID NOs: 1731-1996, 52, 951, 1450, 957, 1452, 1455, 65, 68, 69, 72, 75, 1457, 967, 1458, 970, 971, 974, 1462, 82, 977, 978, 982, 90, 989, 990, 215, 999-1001, 96, 1468, 1005, 1006, 218, 1014, 1018, 1019), are set forth in the SEQUENCE LISTING. The nucleotide sequences of the corresponding 290 genes identified in Table 12, (SEQ ID NOs: 1472-1730, 2, 896, 1429, 902, 1431, 15, 18, 19, 22, 25, 1436, 913, 1437, 916, 917, 920, 1441, 32, 923, 927, 39, 934, 935, 210, 939, 44, 1445, 943, 212, 946, 949), are set forth in the SEQUENCE LISTING.

TABLE 12
PPARγ_3T3L1_Compound_Classifier_{—}
Probe_303 (Species: Mouse Cell Line)

| Accession | | Gene | Probe SEQ |
|---|---|---|---|
| number | Gene Name | SEQ ID NO | ID NO |
| AK005615 | 1700001N19Rik | 1472 | 1731 |
| NM_007760 | Crat | 1473 | 1732 |
| AK013984 | 3110003A17Rik | 1474 | 1733 |
| AW909114 | MGC28611 | 2 | 52 |
| AK003912 | 1110025G12Rik | 1475 | 1734 |
| AK013511 | Ndufv2 | 896 | 951 |
| AK009628 | 2310035C23Rik | 1476 | 1735 |
| NM_021704 | Cxcl12 | 1477 | 1736 |
| AK003232 | Cbr3 | 1478 | 1737 |
| BC002149 | 4633402C03Rik | 1479 | 1738 |
| AK011998 | 2610528M18Rik | 1480 | 1739 |
| AK009071 | 2310001K24Rik | 1481 | 1740 |
| AK016432 | 4931406C07Rik | 1482 | 1741 |
| AK017037 | 4930433D19Rik | 1483 | 1742 |
| BC004645 | Aco2 | 1429 | 1450 |
| NM_011677 | Ung | 1484 | 1743 |
| AK013880 | Nars | 1485 | 1744 |
| NM_010697 | Ldb1 | 1486 | 1745 |
| AK019322 | 2900029G13Rik | 1487 | 1746 |
| NM_011868 | Peci | 1488 | 1747 |
| NM_011921 | Aldh1a7 | 1489 | 1748 |
| NM_025772 | Dtnbp1 | 1490 | 1749 |
| AK004338 | 1110061E11Rik | 1491 | 1750 |
| NM_011031 | P4ha2 | 1492 | 1751 |
| NM_007672 | Cdr2 | 1493 | 1752 |
| NM_015734 | Col5a1 | 1494 | 1753 |
| AK010791 | 2410131K14Rik | 1495 | 1754 |
| NM_011701 | Vim | 1496 | 1755 |
| NM_011050 | Pdcd4 | 1497 | 1756 |
| NM_016861 | Pdlim1 | 1498 | 1757 |
| AK011193 | 2600013D04Rik | 1499 | 1758 |
| NM_020026 | B3galt3 | 1500 | 1759 |
| NM_008768 | Orm1 | 1501 | 1760 |
| AV367848 | AA959574 | 1502 | 1761 |
| AK005869 | 1700011I11Rik | 1503 | 1762 |
| NM_008590 | Mest | 1504 | 1763 |
| BI689765 | AA617265 | 1505 | 1764 |
| AK008764 | 2210021K23Rik | 1506 | 1765 |
| NM_025384 | 1110003P16Rik | 902 | 957 |
| NM_010634 | Fabp5 | 1507 | 1766 |
| AK012054 | 2610319K07Rik | 1508 | 1767 |
| NM_015744 | Enpp2 | 1431 | 1452 |
| AF294617 | Pfkfb3 | 1509 | 1768 |
| AV298518 | AV298518 | 1510 | 1769 |
| AK004987 | Mkks | 1511 | 1770 |
| X15052 | Ncam1 | 1512 | 1771 |
| NM_007473 | Aqp7 | 1513 | 1772 |
| AK007902 | 1810059C13Rik | 1514 | 1773 |
| AK019783 | 4930564I24Rik | 1515 | 1774 |
| BC005552 | Asns | 1516 | 1775 |
| NM_016762 | Matn2 | 1517 | 1776 |
| NM_007881 | Drpla | 1518 | 1777 |
| AK009197 | 2310007D03Rik | 1519 | 1778 |
| AK013761 | 2900070E19Rik | 1520 | 1779 |
| NM_009320 | Slc6a6 | 1521 | 1780 |
| NM_008520 | Ltbp3 | 1522 | 1781 |
| AK004614 | 1200006I17Rik | 1523 | 1782 |
| NM_008638 | Mthfd2 | 1524 | 1783 |
| AK012758 | 1200014I03Rik | 1525 | 1784 |
| NM_011424 | Ncor2 | 1526 | 1785 |
| AK020007 | 5830411O09Rik | 1527 | 1786 |
| AV341581 | 6330577E15Rik | 1528 | 1787 |
| AK008165 | 2010009K05Rik | 1529 | 1788 |
| NM_032398 | Plvap | 1530 | 1789 |
| NM_011693 | Vcam1 | 1531 | 1790 |
| BC003432 | Etfa | 1532 | 1791 |
| AK005710 | Slc25a19 | 1533 | 1792 |
| NM_011641 | Trp63 | 1534 | 1793 |
| AK004743 | Myo1c | 1535 | 1794 |
| NM_009149 | Selel | 1536 | 1795 |
| NM_009058 | Rgds | 1537 | 1796 |
| AK004759 | 1200014F01Rik | 1538 | 1797 |
| AK004153 | 1110038D17Rik | 1539 | 1798 |
| AK010185 | 2310075M15Rik | 1540 | 1799 |
| AK002769 | 0610037F22Rik | 1541 | 1800 |
| AK019459 | Atp5f1 | 1542 | 1801 |
| AF179996 | Sept8 | 1543 | 1802 |
| NM_011462 | Spin | 1544 | 1803 |
| AK017610 | 2810011K15Rik | 1545 | 1804 |
| NM_021893 | Pdcd1lg1 | 1546 | 1805 |
| AK004193 | 1110046O21Rik | 1434 | 1455 |
| BC003988 | Rbm5 | 1547 | 1806 |
| AK009315 | 2310012G06Rik | 15 | 65 |
| AK021117 | C030033M12Rik | 1548 | 1807 |
| AV378562 | 2410022M24Rik | 1549 | 1808 |
| NM_007945 | Eps8 | 1550 | 1809 |
| NM_008608 | Mmp14 | 1551 | 1810 |
| NM_013655 | Cxcl12 | 1552 | 1811 |
| AK003270 | Tbrg1 | 1553 | 1812 |
| AK006810 | 2210018M03Rik | 1554 | 1813 |
| AK005515 | 1600021P15Rik | 1555 | 1814 |
| BB001681 | MICAL-3 | 1556 | 1815 |
| AK021325 | D730003I15Rik | 1557 | 1816 |
| NM_011782 | Adamts5 | 18 | 68 |
| AW120656 | MGC28924 | 1558 | 1817 |
| AK002851 | 0610039N19Rik | 1559 | 1818 |
| NM_011598 | Tlbp | 1560 | 1819 |
| AV075202 | Acadvl | 1561 | 1820 |
| AK013448 | 2810487F15Rik | 1562 | 1821 |
| NM_019729 | Usp8 | 1563 | 1822 |
| NM_020578 | Ehd3 | 19 | 69 |
| BE947541 | BE947541 | 1564 | 1823 |
| AK017403 | 5430437E11Rik | 1565 | 1824 |
| AK004526 | 1810061M12Rik | 1566 | 1825 |
| AK004642 | Lfng | 1567 | 1826 |
| NM_011766 | Zfpm2 | 1568 | 1827 |
| AK010506 | Pbx4 | 1569 | 1828 |
| BB113348 | BB113348 | 1570 | 1829 |
| AK019860 | Agpt2 | 1571 | 1830 |
| AK018466 | 8430436O14Rik | 1572 | 1831 |
| AK013157 | 2810425J22Rik | 1573 | 1832 |
| AK010891 | 2510002J07Rik | 22 | 72 |
| AK002480 | 0610010I13Rik | 1574 | 1833 |
| NM_008735 | Nrip1 | 1575 | 1834 |
| AK007896 | Cdc42ep1 | 1576 | 1835 |
| NM_015757 | Pcdh13 | 1577 | 1836 |
| AW476152 | Adamts2 | 1578 | 1837 |
| NM_007941 | Epim | 1579 | 1838 |
| AK011976 | Angptl2 | 1580 | 1839 |
| AK007873 | 1810055P05Rik | 1581 | 1840 |
| AK004732 | 1200013A08Rik | 25 | 75 |
| NM_021528 | C4st2-pending | 1582 | 1841 |
| AK009739 | Klf15 | 1583 | 1842 |
| AK014643 | 4733401N06Rik | 1584 | 1843 |
| AV221349 | ri\|3322401K10\| | 1585 | 1844 |
| | PX00010E04\|\|2295 | | |
| AK004659 | Cf12 | 1586 | 1845 |
| AK007497 | 1810014L12Rik | 1436 | 1457 |
| AK004770 | 9130009D18Rik | 1587 | 1846 |
| NM_023294 | 2610020P18Rik | 1588 | 1847 |
| AK004670 | 1200009F10Rik | 1589 | 1848 |
| NM_023058 | Pkmyt1-pending | 1590 | 1849 |
| BI101760 | AW214504 | 1591 | 1850 |
| AK011889 | 2610205H19Rik | 1592 | 1851 |
| NM_011812 | Fbln5 | 1593 | 1852 |
| NM_008216 | Has2 | 1594 | 1853 |
| AK003283 | Mrpl13 | 913 | 967 |
| NM_007705 | Cirbp | 1595 | 1854 |
| NM_025892 | 1500031L02Rik | 1596 | 1855 |
| NM_024207 | 1110021N07Rik | 1437 | 1458 |
| AK002277 | Igfbp7 | 1597 | 1856 |
| NM_008564 | Mcmd2 | 1598 | 1857 |
| AV102233 | AV102233 | 1599 | 1858 |
| NM_008486 | Anpep | 1600 | 1859 |
| BC002107 | D5Ertd371e | 1601 | 1860 |
| NM_007970 | Ezh1 | 1602 | 1861 |
| AK002744 | 0610033L03Rik | 1603 | 1862 |
| AK017684 | 5730466C23Rik | 1604 | 1863 |
| AK003387 | Ube2g2 | 1605 | 1864 |
| AK002942 | 0610020I02Rik | 1606 | 1865 |
| NM_010225 | Foxf2 | 1607 | 1866 |
| AV077222 | 2810422B09Rik | 1608 | 1867 |
| AK007959 | Klf3 | 1609 | 1868 |
| AK021144 | C030044C12Rik | 1610 | 1869 |
| BF160060 | AV212693 | 1611 | 1870 |
| NM_025910 | 1810047J07Rik | 1612 | 1871 |
| AV247986 | Dysf | 1613 | 1872 |
| AK017918 | 5830411H19Rik | 1614 | 1873 |
| AK005080 | Suclg1 | 916 | 970 |
| AW490567 | Jag1 | 1615 | 1874 |
| AV238629 | AV238629 | 1616 | 1875 |
| AK006128 | Abcc3 | 1617 | 1876 |
| AK002889 | 0610041L09Rik | 917 | 971 |
| AK018089 | 6230416A05Rik | 1618 | 1877 |
| NM_008810 | Pdha1 | 1619 | 1878 |
| NM_025626 | 3110001A13Rik | 1620 | 1879 |
| AF096898 | D15Mit260 | 1621 | 1880 |
| AK003535 | 1110007F12Rik | 1622 | 1881 |
| NM_023644 | Mccc1 | 1623 | 1882 |
| AK008125 | 2010005I16Rik | 1624 | 1883 |
| BC004702 | Birc5 | 1625 | 1884 |
| BE553640 | 1700084G18Rik | 1626 | 1885 |
| AJ276796 | Cars | 1627 | 1886 |
| NM_019804 | B4galt4 | 1628 | 1887 |
| AK008255 | 2010015J01Rik | 1629 | 1888 |
| NM_011796 | Capn10 | 1630 | 1889 |
| AK004851 | 1300002F13Rik | 1631 | 1890 |
| NM_007620 | Cbr1 | 1632 | 1891 |
| AK010706 | 2410055N02Rik | 1633 | 1892 |
| AK008822 | 4933404O11Rik | 1634 | 1893 |
| NM_010918 | Nktr | 1635 | 1894 |
| AK002320 | 0610008C08Rik | 920 | 974 |
| NM_009104 | Rrm2 | 1636 | 1895 |
| BC004801 | LOC207933 | 1637 | 1896 |
| AK009291 | 2310011D08Rik | 1638 | 1897 |
| NM_010422 | Hexb | 1639 | 1898 |
| AK013062 | 2810410A03Rik | 1640 | 1899 |
| AK003556 | 2310075G14Rik | 1641 | 1900 |
| NM_016788 | Tnk2 | 1642 | 1901 |
| NM_007707 | Cish3 | 1643 | 1902 |
| NM_016897 | Timm23 | 1441 | 1462 |
| NM_016810 | Gosr1 | 1644 | 1903 |
| AK016659 | 4933405A16Rik | 1645 | 1904 |
| AK020118 | 6720429C22Rik | 1646 | 1905 |
| AK020182 | 7330412A13Rik | 1647 | 1906 |
| AK011182 | 2600010N21Rik | 1648 | 1907 |
| NM_009378 | Thbd | 1649 | 1908 |
| AK007856 | 1810054D07Rik | 1650 | 1909 |
| NM_024223 | Crip2 | 1651 | 1910 |
| AK020048 | 6030408B16Rik | 1652 | 1911 |
| AK019002 | 1810004I06Rik | 1653 | 1912 |
| AK013740 | 6530401D17Rik | 32 | 82 |
| AK010344 | 2410002L19Rik | 1654 | 1913 |
| NM_011479 | Sptlc2 | 1655 | 1914 |
| AK003709 | 1110014L14Rik | 1656 | 1915 |
| NM_025809 | 1200003C23Rik | 1657 | 1916 |
| AK008679 | 2210008N01Rik | 1658 | 1917 |
| AK003975 | 1500004O06Rik | 923 | 978 |
| | | | 977 |
| AK010747 | 2410089E03Rik | 1659 | 1918 |
| NM_026473 | 2310057H16Rik | 1660 | 1919 |
| NM_008910 | Ppm1a | 1661 | 1920 |
| AK003621 | 1110012D08Rik | 1662 | 1921 |
| AK004432 | 1190001I08Rik | 1663 | 1922 |
| AK018500 | 2700038I16Rik | 1664 | 1923 |
| AK016881 | 4933424A20Rik | 1665 | 1924 |
| NM_026842 | Ubqln1 | 1666 | 1925 |
| BC004020 | BC004020 | 927 | 982 |
| AK002699 | Ptk9l | 1667 | 1926 |
| NM_008841 | Pik3r2 | 1668 | 1927 |
| NM_016812 | Banp | 1669 | 1928 |
| BC003261 | Stk5 | 1670 | 1929 |
| AK003995 | 1110030N17Rik | 1671 | 1930 |
| NM_007996 | Fdx1 | 1672 | 1931 |
| NM_013792 | Naglu | 1673 | 1932 |
| AC002397 | CD4, A-2, B, GNB3, | 1674 | 1933 |
| | C8, ISOT, TPI, B7, | | |
| | ENO2, DRPLA, U7snRNA, | | |
| | C10, PTPN6, BAP, C2F | | |
| NM_017370 | Hp | 1675 | 1934 |
| AK010043 | 2310065E01Rik | 1676 | 1935 |
| BC003908 | 2310046B19Rik | 1677 | 1936 |
| NM_007609 | Casp11 | 1678 | 1937 |
| BE994229 | Tcfcp2 | 1679 | 1938 |
| NM_008055 | Fzd4 | 1680 | 1939 |
| AK003586 | 1110008K06Rik | 1681 | 1940 |
| AK013580 | 2900024C23Rik | 1682 | 1941 |
| BC004633 | 2410011G03Rik | 1683 | 1942 |
| AK009883 | Atp5g1 | 1684 | 1943 |
| AK010765 | Bag4 | 1685 | 1944 |
| AK002531 | Sat | 1686 | 1945 |
| AK016103 | 4930553F04Rik | 39 | 90 |
| BC003766 | Nfix | 1687 | 1946 |
| BC010825 | 1700112L09Rik | 1688 | 1947 |
| U03419 | Col1a1 | 1689 | 1948 |
| U03715 | Col18a1 | 1690 | 1949 |
| M20497 | Fabp4 | 1691 | 1950 |
| AA543477 | Mgst1 | 1692 | 1951 |
| Z38015 | DM-PK | 1693 | 1952 |
| X01756 | Cycs | 934 | 989 |
| L02331 | Sult1a1 | 1694 | 1953 |
| BC007148 | Vps26 | 1695 | 1954 |
| AF013262 | Lum | 1696 | 1955 |
| BC009134 | AA959601 | 935 | 990 |
| BC008989 | LOC217166 | 1697 | 1956 |
| M13264 | Fabp4 | 210 | 215 |
| Z71189 | Acadvl | 939 | 1001 |
| | | | 999 |
| | | | 1000 |
| AF007267 | Pmm1 | 1698 | 1957 |
| AF011450 | Col15a1 | 1699 | 1958 |
| AF057286 | Epn2 | 1700 | 1959 |
| D01093 | Pcsk4 | 1701 | 1960 |
| D86949 | Plxna2 | 1702 | 1961 |
| J04632 | Gstm1 | 44 | 96 |
| J04696 | Gstm2 | 1703 | 1962 |
| L02918 | Col5a2 | 1704 | 1963 |
| L57509 | Ddr1 | 1705 | 1964 |
| M16229 | Mor1 | 1445 | 1468 |
| M18194 | Fn1 | 1706 | 1965 |
| M32240 | Pmp22 | 1707 | 1966 |
| | | | 1967 |
| | | | 1968 |
| M93275 | Adfp | 943 | 1005 |
| | | | 1006 |
| U01841 | Pparg | 212 | 1969 |
| | | | 1970 |
| | | | 218 |
| U03283 | Cyp1b1 | 1708 | 1971 |
| | | | 1972 |
| U08020 | Col1a1 | 1709 | 1973 |
| U14332 | Il15 | 1710 | 1974 |
| U21489 | Acadl | 946 | 1014 |
| U43298 | Lamb3 | 1711 | 1975 |
| U58883 | Sorbs1 | 1712 | 1976 |
| | | | 1977 |
| U67187 | Rgs2 | 1713 | 1978 |
| U79550 | Snai2 | 1714 | 1979 |
| X04017 | Sparc | 1715 | 1980 |
| X04367 | Pdgfrb | 1716 | 1981 |
| | | | 1982 |
| X63535 | Axl | 1717 | 1983 |
| X67469 | Lrp1 | 1718 | 1984 |
| X89998 | Hsd17b4 | 949 | 1018 |
| | | | 1019 |
| Y15163 | Cited2 | 1719 | 1985 |
| J03484 | Lamc1 | 1720 | 1986 |
| X04972 | Sod2 | 1721 | 1987 |
| X69620 | Inhbb | 1722 | 1988 |
| AI314880 | Tstap91a | 1723 | 1989 |
| AI746433 | A1746433 | 1724 | 1990 |
| U70139 | Ccr4 | 1725 | 1991 |
| AB023957 | EIG180 | 1726 | 1992 |
| NM_011513 | Surf5 | 1727 | 1993 |
| NM_010284 | Ghr | 1728 | 1994 |
| AI448406 | AI562151 | 1729 | 1995 |
| AI449447 | AI449447 | 1730 | 1996 |

The average of the logRatio of each of the 303 probes (SEQ ID NOs: 1731-1996, 52, 951, 1450, 957, 1452, 1455, 65, 68, 69, 72, 75, 1457, 967, 1458, 970, 971, 974, 1462, 82, 977, 978, 982, 90, 989, 990, 215, 999-1001, 96, 1468, 1005, 1006, 218, 1014, 1018, 1019) in Group 1 was calculated and served as the template. A classifier value for a PPARγ agonist, or partial agonist, was calculated in the following manner. The value (expressed as a percentage) of the logRatio divided by the template logRatio for each of the 303 probes (SEQ ID NOs: 1731-1996, 52, 951, 1450, 957, 1452, 1455, 65, 68, 69, 72, 75, 1457, 967, 1458, 970, 971, 974, 1462, 82, 977, 978, 982, 90, 989, 990, 215, 999-1001, 96, 1468, 1005, 1006, 218, 1014, 1018, 1019) was calculated, and then the mean of the resulting 303 percentages was calculated. This mean value was the classifier value for the PPARγ agonist, or partial agonist.

Table 13 below shows the classifier value for the compounds that were tested in Phase 3 of the 3T3L1 experiment.

TABLE 13

| Compound | Classifier Value |
|---|---|
| Agonist 1 | 0.881 |
| Agonist 5-(4-{2-[methyl(pyridin-2- | 0.850 |
| yl)amino]ethoxy}benzyl)-1,3- | |
| thiazolidine-2,4-dione) | |
| Partial agonist 16 | 0.708 |
| Partial agonist 15 | 0.651 |
| Partial agonist 17 | 0.550 |
| Partial agonist 4 | 0.473 |
| Partial agonist 10 | 0.387 |
| Partial agonist 13 | 0.363 |
| Partial agonist 9 | 0.352 |
| Partial agonist 12 | 0.350 |
| Partial agonist | 0.341 |
| (2R)-2-(4-chloro-3-{[3-(6- | |
| methoxy-1,2-benzisoxazol-3-yl)-2- | |
| methyl-6-(trifluoromethoxy)-1H-indol-1- | |
| yl]methyl}phenoxy)propanoate | |
| Partial agonist 11 | 0.309 |
| Partial agonist 14 | 0.302 |
| PPARα agonist | 0.096 |

This classifier gene population is useful for ranking candidate partial agonists of PPARγ and full agonists of PPARγ relative to one or more known partial agonists of PPARγ and one or more known full agonists of PPARγ.

### EXAMPLE 4

This Example describes the identification of a population of genes that yield an expression pattern that correlates with the stimulation of PPARα receptors by an agent. This population of genes can be used, for example, to screen candidate PPARγ agonists, or partial agonists, to identify those candidate agents that possess the undesirable property of stimulating PPARα receptors. This population of genes can also be used, for example, to identify PPARα agonists, or PPARα partial agonists.

Wild type mice, and mice that had been genetically modified to inactivate all copies of the gene encoding the PPARα protein (called PPARα knockout mice), were treated with PPARα agonists. Genes whose expression was significantly affected in wild type mice in response to the PPARα agonists, but which was not significantly affected in PPARα knockout mice, were identified. The resulting gene set was considered a PPARα receptor-dependent signature gene set.

Two PPARα agonists were orally administered to wild type mice (abbreviated as WT mice) and to PPARα knockout mice (abbreviated as KO mice). The two compounds were Fenofibrate (administered at a dosage of 200 milligrams per kilogram body weight), and [4-chloro-6-(2,3-xylidino)-2-pyrimidinylthio]acetic acid (administered at a dosage of 30 milligrams per kilogram body weight). The PPARα agonists were administered at day 1 and day 7. Three experimental conditions were tested for each PPARα agonist:
WT control pool vs. WT treatment (hereafter WT vs. WT treatment)
KO control pool vs. KO treatment (hereafter KO vs. KO treatment)
WT treatment vs. KO treatment (hereafter WT treatment vs. KO treatment)

The hybrid ANOVA method described in Example 1 was used to calculate the ANOVA-pvalue and the average of logRatio of gene expression for each gene in each of the 12 experimental groups (i.e., two drug treatments×two time points×three conditions). Signature genes were identified that had an ANOVA-pvalue less than 0.01, and the absolute value of the average of logRatio greater than log₁₀1.5.

The union of the one day signature genes with the seven day signature genes for each of the two PPARα: agonist treatments under each of the three experimental conditions (WT vs. WT treatment; KO vs. KO treatment; WT treatment vs. KO treatment) was used to identify genes whose expression was significantly regulated in the WT vs. WT treatment, and WT treatment vs. KO treatment groups, but not in the KO vs. KO treatment group, for each of the two PPARα agonist treatments. The genes that were common to the PPARα agonist treatments were identified, thereby yielding a total of 978 probes as identified in Table 14, (SEQ ID NOs: 2796-3683, 1732, 1734, 53, 1740, 1449, 1450, 1747, 1748, 1037, 1759, 957, 1774, 60, 1780, 63, 1797, 962, 1808, 1041, 1809, 1817, 1818, 1820, 1824, 71, 72, 1833, 966, 1873, 970-973, 1879, 1046, 1047, 976, 1898, 1904, 80, 1910, 86, 1932, 1933, 1941, 1049, 989, 1953, 991-993, 1050, 1051, 994, 215, 216, 93, 94, 998-1001, 1465-1467, 1957, 1002, 214, 1962, 1005-1007, 1056, 1057, 1009-1014, 1974, 1975, 1977, 1979, 1016-1019, 1994, 101), corresponding to 870 unique genes as identified in Table 14, (SEQ ID NOs: 1997-2795, 1473, 1475, 3, 1481, 1429, 1488, 1489, 1021, 1500, 902, 1515, 10, 1521, 13, 1538, 908, 1549, 1025, 1550, 1558, 1559, 1561, 1565, 21, 22, 1574, 912, 1614, 916-919, 1620, 1030, 1031, 922, 1639, 1645, 30, 1651, 35, 1673, 1674, 1682, 1033, 934, 1694, 936, 1034, 937, 210, 42, 939, 1444, 1698, 940, 209, 1703, 943, 1035, 945, 1710, 946, 1711, 1712, 1714, 948, 949, 142, 1728, 49).

TABLE 14
PPARα_3T3L1_Liver_Depended_Regulation_Probe_978
(Species: Mouse Cell Line)

| Accession | Gene | Gene SEQ | Probe SEQ |
|---|---|---|---|
| number | Name | ID NO | ID NO |
| AK005570 | 1600032L17Rik | 1997 | 2796 |
| NM_008298 | Dnaja1 | 1998 | 2797 |
| AW122190 | AW122190 | 1999 | 2798 |
| AK018646 | 9130022K13Rik | 2000 | 2799 |
| AK020256 | 9030616G12Rik | 2001 | 2800 |
| AK012001 | 2610306P15Rik | 2002 | 2801 |
| AV225723 | AA408038 | 2003 | 2802 |
| AK012577 | 2700087I09Rik | 2004 | 2803 |
| AK015314 | 0710001P09Rik | 2005 | 2804 |
| NM_019926 | Mtm1 | 2006 | 2805 |
| BE691027 | BE691027 | 2007 | 2806 |
| AK019063 | 2210408B16Rik | 2008 | 2807 |
| AK005808 | 1700010A17Rik | 2009 | 2808 |
| AV269843 | MGC30495 | 2010 | 2809 |
| AK014452 | 3830422K02Rik | 2011 | 2810 |
| NM_019723 | Slc22a9 | 2012 | 2811 |
| BC011492 | 9130020G10Rik | 2013 | 2812 |
| AI449628 | AI449595 | 2014 | 2813 |
| BC004092 | Nd1-pending | 2015 | 2814 |
| NM_007760 | Crat | 1473 | 1732 |
| | | | 2815 |
| BF455494 | BF455494 | 2016 | 2816 |
| NM_021526 | Poh1-pending | 2017 | 2817 |
| AK012370 | Scd1 | 2018 | 2818 |
| AK012685 | 2810007J24Rik | 2019 | 2819 |
| AK019713 | 4930529O08Rik | 2020 | 2820 |
| AK015561 | 4930472G13Rik | 2021 | 2821 |
| AK007857 | 1810054F20Rik | 2022 | 2822 |
| NM_028119 | 2610043A19Rik | 2023 | 2823 |
| AK015340 | 4930439B20Rik | 2024 | 2824 |
| NM_010139 | Epha2 | 2025 | 2825 |
| AK002693 | Dgat2l1 | 2026 | 2826 |
| AK016318 | 4930579F01Rik | 2027 | 2827 |
| AK013414 | Sip1 | 2028 | 2828 |
| NM_027288 | 2410030O07Rik | 2029 | 2829 |
| BC002151 | 1110056N09Rik | 2030 | 2830 |
| AK009210 | 2310007J06Rik | 2031 | 2831 |
| AV356694 | AV356694 | 2032 | 2832 |
| AK005622 | Insl6 | 2033 | 2833 |
| AK009377 | 2310016C08Rik | 2034 | 2834 |
| AK003912 | 1110025G12Rik | 1475 | 1734 |
| BB541540 | Clcn2 | 2035 | 2835 |
| NM_025558 | 1810044O22Rik | 2036 | 2836 |
| NM_008543 | Madh7 | 3 | 53 |
| NM_011596 | Atp6vOa2 | 2037 | 2837 |
| AF339106 | Foxp2 | 2038 | 2838 |
| AK003879 | 5730512J02Rik | 2039 | 2839 |
| NM_008878 | Serpinf2 | 2040 | 2840 |
| NM_018760 | Slc4a4 | 2041 | 2841 |
| NM_008129 | Gclm | 2042 | 2842 |
| AK013628 | 2900040J22Rik | 2043 | 2843 |
| NM_008681 | Ndrl | 2044 | 2844 |
| BF579112 | AW121759 | 2045 | 2845 |
| AK009071 | 2310001K24Rik | 1481 | 1740 |
| AK017628 | 5730438N18Rik | 2046 | 2846 |
| AK012088 | Facl3 | 2047 | 2847 |
| NM_026586 | 6720475J19Rik | 2048 | 2848 |
| NM_007930 | Enc1 | 2049 | 2849 |
| AK009134 | Acyp2 | 2050 | 2850 |
| BC004645 | Aco2 | 1429 | 1449 |
| | | | 1450 |
| | | | 2851 |
| AV278562 | AV278562 | 2051 | 2852 |
| AK018792 | 1520401O13Rik | 2052 | 2853 |
| AK010547 | 5730471K09Rik | 2053 | 2854 |
| NM_010237 | Frk | 2054 | 2855 |
| AK014380 | 3321402G02Rik | 2055 | 2856 |
| NM_010001 | Cyp2c37 | 2056 | 2857 |
| NM_009794 | Capn2 | 2057 | 2858 |
| AK005616 | 1700001O02Rik | 2058 | 2859 |
| NM_027280 | Nkd1 | 2059 | 2860 |
| AK013597 | 2900026A02Rik | 2060 | 2861 |
| AK004307 | Grhpr | 2061 | 2862 |
| NM_008253 | Hmgb3 | 2062 | 2863 |
| AK008360 | Fcgrt | 2063 | 2864 |
| AK009343 | 2310014L03Rik | 2064 | 2865 |
| AV115239 | AV115239 | 2065 | 2866 |
| NM_008769 | Otc | 2066 | 2867 |
| AK004782 | Lgals8 | 2067 | 2868 |
| AK011596 | Trfr | 2068 | 2869 |
| NM_011868 | Peci | 1488 | 1747 |
| AK006140 | 1700020A13Rik | 2069 | 2870 |
| W29450 | AA410048 | 2070 | 2871 |
| BC004728 | BC004728 | 2071 | 2872 |
| AL359935 | LOC209798 | 2072 | 2873 |
| BG970486 | ri\|1700025L02\| | 2073 | 2874 |
| | ZX00037H10\|\|1579 | | |
| BC005759 | Secl412 | 2074 | 2875 |
| NM_011921 | Aldh1a7 | 1489 | 1748 |
| AK016187 | 4930562A09Rik | 2075 | 2876 |
| AK003420 | 1110004G24Rik | 2076 | 2877 |
| NM_023805 | Slc38a3 | 2077 | 2878 |
| AK018155 | 6330410P18Rik | 2078 | 2879 |
| AK004550 | 1200002M06Rik | 2079 | 2880 |
| AK013094 | 2810416A17Rik | 2080 | 2881 |
| NM_018743 | LOC55933 | 2081 | 2882 |
| AW456595 | AW456595 | 2082 | 2883 |
| AK020668 | 1200007B05Rik | 2083 | 2884 |
| NM_007437 | Aldh3a2 | 2084 | 2885 |
| NM_010437 | Hivep2 | 2085 | 2886 |
| NM_007706 | Cish2 | 2086 | 2887 |
| AK017063 | 4933435A13Rik | 2087 | 2888 |
| AV278924 | ri\|4933404M19\| | 2088 | 2889 |
| | PX00019F10\|\|1119 | | |
| NM_008303 | Hspe1 | 1021 | 1037 |
| AK003228 | 1110001I14Rik | 2089 | 2890 |
| NM_022880 | Slc29a1 | 2090 | 2891 |
| AK005033 | D7Ertd753e | 2091 | 2892 |
| NM_010497 | Idh1 | 2092 | 2893 |
| AB051827 | Arhu | 2093 | 2894 |
| NM_026172 | Decr1 | 2094 | 2895 |
| AK014017 | Egfr | 2095 | 2896 |
| NM_010324 | Got1 | 2096 | 2897 |
| NM_011066 | Per2 | 2097 | 2898 |
| AK004305 | D10Ertd749e | 2098 | 2899 |
| AK020922 | Pde6h | 2099 | 2900 |
| NM_009381 | Thrsp | 2100 | 2901 |
| NM_009016 | Raet1a | 2101 | 2902 |
| NM_025545 | Aptx | 2102 | 2903 |
| NM_008382 | Inhbe | 2103 | 2904 |
| NM_030262 | BC003494 | 2104 | 2905 |
| BB312353 | BB312353 | 2105 | 2906 |
| AK007138 | 2810433K01Rik | 2106 | 2907 |
| AK017354 | 5430428G01Rik | 2107 | 2908 |
| AK016991 | 4933430F16Rik | 2108 | 2909 |
| NM_011020 | Osp94 | 2109 | 2910 |
| NM_019447 | Hgfac | 2110 | 2911 |
| NM_020026 | B3galt3 | 1500 | 1759 |
| AK004138 | 1110037D04Rik | 2111 | 2912 |
| AK004650 | 1200008D14Rik | 2112 | 2913 |
| NM_008331 | Ifit1 | 2113 | 2914 |
| AI551079 | Cyp4a12 | 2114 | 2915 |
| AK002555 | D18Ertd240e | 2115 | 2916 |
| NM_025566 | 2600017J23Rik | 2116 | 2917 |
| AK002477 | Tm4sfl1 | 2117 | 2918 |
| BF322562 | Copbl | 2118 | 2919 |
| BB561321 | BB561321 | 2119 | 2920 |
| AK014658 | 4833406M21Rik | 2120 | 2921 |
| AK020935 | A930036K24Rik | 2121 | 2922 |
| AK004600 | Arhgef3 | 2122 | 2923 |
| NM_016808 | Usp2 | 2123 | 2924 |
| NM_015818 | Hs6st1 | 2124 | 2925 |
| NM_025384 | 1110003P16Rik | 902 | 957 |
| NM_019781 | Pex14 | 2125 | 2926 |
| NM_010867 | Myom1 | 2126 | 2927 |
| AF288783 | Pyg1 | 2127 | 2928 |
| AK008330 | 2010107C10Rik | 2128 | 2929 |
| NM_008260 | Foxa3 | 2129 | 2930 |
| NM_010707 | Lgals6 | 2130 | 2931 |
| AI849720 | Ndst1 | 2131 | 2932 |
| NM_011967 | Psma5 | 2132 | 2933 |
| AK003902 | 1110021L09Rik | 2133 | 2934 |
| NM_009289 | Stk2 | 2134 | 2935 |
| AK012110 | 2610511G02Rik | 2135 | 2936 |
| AK010754 | 2410091N08Rik | 2136 | 2937 |
| NM_032400 | Gpr91 | 2137 | 2938 |
| AK021023 | B430311C09Rik | 2138 | 2939 |
| BB557066 | BB557066 | 2139 | 2940 |
| BC004781 | BC004781 | 2140 | 2941 |
| AK004768 | Osbpl3 | 2141 | 2942 |
| NM_025591 | 2010309E21Rik | 2142 | 2943 |
| AK019783 | 4930564I24Rik | 1515 | 1774 |
| AK006955 | 1700080G11Rik | 2143 | 2944 |
| AK013642 | 2900042M13Rik | 2144 | 2945 |
| NM_023143 | C1r | 2145 | 2946 |
| NM_019758 | Mtch2-pending | 2146 | 2947 |
| BE691256 | 2010004B12Rik | 2147 | 2948 |
| BC003488 | Lmo4 | 2148 | 2949 |
| AK021389 | 2610511G02Rik | 2149 | 2950 |
| BB463934 | 1200006P13Rik | 2150 | 2951 |
| AK010472 | 2410012H22Rik | 2151 | 2952 |
| AK005060 | 1300019H02Rik | 2152 | 2953 |
| AK004287 | 1110057L18Rik | 2153 | 2954 |
| AK018458 | 8430436A10Rik | 2154 | 2955 |
| AK006159 | 1700020G04Rik | 2155 | 2956 |
| AK004926 | Igfals | 2156 | 2957 |
| AK013959 | Trim13 | 2157 | 2958 |
| AF304306 | Hsd17b11 | 2158 | 2959 |
| AK004934 | 1300007L22Rik | 2159 | 2960 |
| AK007710 | 1810036L03Rik | 2160 | 2961 |
| AV279434 | 4930458D05Rik | 10 | 60 |
| AK017766 | 5730512J02Rik | 2161 | 2962 |
| NM_009320 | Slc6a6 | 1521 | 1780 |
| AK014728 | 4833419J07Rik | 2162 | 2963 |
| AK014047 | 3110013K01Rik | 2163 | 2964 |
| BB429858 | BB429858 | 2164 | 2965 |
| AK011567 | 2610027H17Rik | 2165 | 2966 |
| NM_030611 | Hsd17b5 | 2166 | 2967 |
| NM_009444 | Tgoln2 | 2167 | 2968 |
| AW743226 | AW743226 | 2168 | 2969 |
| NM_011201 | Ptpn1 | 2169 | 2970 |
| AK012041 | Ris2 | 2170 | 2971 |
| AK011544 | 1500031M22Rik | 2171 | 2972 |
| BB556229 | 2310015N21Rik | 2172 | 2973 |
| AK014518 | Hal | 2173 | 2974 |
| AK020424 | 9430019C24Rik | 2174 | 2975 |
| AK011578 | Pinx1-pending | 2175 | 2976 |
| AK011605 | Mrpl45 | 2176 | 2977 |
| NM_019992 | Brdg1-pending | 2177 | 2978 |
| AK003434 | Rbpms | 2178 | 2979 |
| BB131710 | BB131710 | 2179 | 2980 |
| AK002718 | Oprs1 | 2180 | 2981 |
| AK009386 | 2310016F22Rik | 2181 | 2982 |
| NM_017380 | 9-Sep | 2182 | 2983 |
| NM_007647 | Entpd5 | 2183 | 2984 |
| NM_009799 | Car1 | 2184 | 2985 |
| NM_016974 | Dbp | 2185 | 2986 |
| AK005032 | 1300017E09Rik | 2186 | 2987 |
| AK021388 | E130114A11Rik | 2187 | 2988 |
| AK003418 | 1110004G14Rik | 2188 | 2989 |
| NM_021548 | Arpp19-pending | 2189 | 2990 |
| AK002217 | 0610005C13Rik | 2190 | 2991 |
| NM_011825 | Prdc-pending | 2191 | 2992 |
| AK005781 | 1700008N02Rik | 2192 | 2993 |
| AK013950 | 3110001I22Rik | 2193 | 2994 |
| AK015354 | Optn | 2194 | 2995 |
| AK003939 | 1110028A07Rik | 2195 | 2996 |
| NM_010892 | Nek2 | 2196 | 2997 |
| AK021082 | C030014O09Rik | 2197 | 2998 |
| BB299566 | BB299566 | 2198 | 2999 |
| AK015050 | 4930402H24Rik | 2199 | 3000 |
| NM_021507 | Sqrdl | 2200 | 3001 |
| NM_023431 | 9430059D04Rik | 2201 | 3002 |
| NM_023160 | Cml1 | 2202 | 3003 |
| AK004867 | 1300002P22Rik | 13 | 63 |
| AK002437 | 0610009O20Rik | 2203 | 3004 |
| BC006074 | 1110018G07Rik | 2204 | 3005 |
| AK002772 | 1500036F01Rik | 2205 | 3006 |
| AK005035 | 1300017J02Rik | 2206 | 3007 |
| AF241249 | 1110033G01Rik | 2207 | 3008 |
| AJ131870 | Atp2a2 | 2208 | 3009 |
| NM_031396 | Cnnm1 | 2209 | 3010 |
| NM_010189 | Fcgrt | 2210 | 3011 |
| NM_011396 | Slc22a5 | 2211 | 3012 |
| | | | 3013 |
| | | | 3014 |
| AV021580 | 4922501H04Rik | 2212 | 3015 |
| AK018177 | Unc5h2 | 2213 | 3016 |
| AK007678 | 1810033A06Rik | 2214 | 3017 |
| AK004759 | 1200014F01Rik | 1538 | 1797 |
| AK011406 | 2610016A03Rik | 2215 | 3018 |
| AK006138 | 1700019P01Rik | 2216 | 3019 |
| AK012473 | 2700063E05Rik | 2217 | 3020 |
| NM_031192 | Ren1 | 2218 | 3021 |
| AV268127 | MGC36416 | 2219 | 3022 |
| NM_025827 | 1300002A08Rik | 2220 | 3023 |
| AK010382 | 2410004E01Rik | 2221 | 3024 |
| AK020283 | 9130219B18Rik | 2222 | 3025 |
| BB568823 | 2210414H16Rik | 2223 | 3026 |
| AK004660 | Abcd3 | 2224 | 3027 |
| AK013812 | 2900083I11Rik | 2225 | 3028 |
| AK003873 | 1110020M10Rik | 2226 | 3029 |
| AK012785 | Pxf | 2227 | 3030 |
| NM_025661 | Ormdl3 | 2228 | 3031 |
| AK018462 | 8430436I03Rik | 2229 | 3032 |
| NM_021304 | Abhd1 | 2230 | 3033 |
| BC004668 | Hps4 | 2231 | 3034 |
| M64404 | Il1rn | 2232 | 3035 |
| NM_026232 | 4933433D23Rik | 2233 | 3036 |
| NM_016669 | Crym | 2234 | 3037 |
| BE987053 | BE987053 | 2235 | 3038 |
| AK015509 | 4930465M17Rik | 2236 | 3039 |
| AK014531 | Palmd | 2237 | 3040 |
| AK018084 | 6230410J09Rik | 2238 | 3041 |
| NM_023465 | Catnbip1 | 2239 | 3042 |
| AK011759 | 2610043O12Rik | 2240 | 3043 |
| AK010209 | 2310076O21Rik | 2241 | 3044 |
| NM_022985 | Awp1-pending | 2242 | 3045 |
| AK016295 | 4930577M16Rik | 2243 | 3046 |
| AF173639 | AI197390 | 2244 | 3047 |
| NM_007980 | Fabp2 | 2245 | 3048 |
| AK002483 | 0610010I20Rik | 908 | 962 |
| AK021270 | C530009C10Rik | 2246 | 3049 |
| AK014111 | Hhex | 2247 | 3050 |
| AK007296 | 1700127B04Rik | 2248 | 3051 |
| AK011417 | Pov1 | 2249 | 3052 |
| AV378562 | 2410022M24Rik | 1549 | 1808 |
| NM_010004 | Cyp2c40 | 2250 | 3053 |
| NM_022983 | Edg7 | 2251 | 3054 |
| NM_019975 | Hpcl-pending | 1025 | 1041 |
| NM_007945 | Eps8 | 1550 | 1809 |
| AV174028 | Bace | 2252 | 3055 |
| AI430696 | Peg3 | 2253 | 3056 |
| NM_013837 | Tpst1 | 2254 | 3057 |
| AI266962 | Cml1 | 2255 | 3058 |
| NM_013484 | C2 | 2256 | 3059 |
| NM_007994 | Fbp2 | 2257 | 3060 |
| | | | 3061 |
| | | | 3062 |
| NM_013545 | Hcph | 2258 | 3063 |
| AK010430 | Ddah1 | 2259 | 3064 |
| AK012478 | 2700063L20Rik | 2260 | 3065 |
| AK008965 | Agpat3 | 2261 | 3066 |
| NM_013731 | Sgk2 | 2262 | 3067 |
| AK007574 | Fgf21 | 2263 | 3068 |
| AK013765 | Ecgf1 | 2264 | 3069 |
| NM_011933 | Decr2 | 2265 | 3070 |
| NM_010391 | H2-Q10 | 2266 | 3071 |
| | | | 3072 |
| | | | 3073 |
| AK004956 | 1300010F03Rik | 2267 | 3074 |
| AK014740 | 4833420O05Rik | 2268 | 3075 |
| AK014558 | 4632408A20Rik | 2269 | 3076 |
| AW120656 | MGC28924 | 1558 | 1817 |
| AK002851 | 0610039N19Rik | 1559 | 1818 |
| AK004204 | 1110048P06Rik | 2270 | 3077 |
| NM_009364 | Tfpi2 | 2271 | 3078 |
| AV075202 | Acadvl | 1561 | 1820 |
| BC003258 | BC003323 | 2272 | 3079 |
| NM_028094 | 2010321J07Rik | 2273 | 3080 |
| BB641340 | ri\|A930014C21\| | 2274 | 3081 |
| | PX00066C21\|\|1837 | | |
| NM_010512 | Igf1 | 2275 | 3082 |
| | | | 3083 |
| NM_007405 | Adcy6 | 2276 | 3084 |
| NM_020009 | Frap1 | 2277 | 3085 |
| AK017403 | 5430437E11Rik | 1565 | 1824 |
| BC004083 | Htatip2 | 2278 | 3086 |
| BB229969 | BB229969 | 2279 | 3087 |
| AV280352 | AV280352 | 21 | 71 |
| BF532887 | ri\|6330415L08\| | 2280 | 3088 |
| | PX00008D23\|\|2975 | | |
| NM_011706 | Trpv2 | 2281 | 3089 |
| AK009125 | 2310003N14Rik | 2282 | 3090 |
| AK013267 | 2810439F02Rik | 2283 | 3091 |
| AK010969 | Psmd4 | 2284 | 3092 |
| AK013874 | 3010001A07Rik | 2285 | 3093 |
| AK011778 | 2610100B16Rik | 2286 | 3094 |
| AK017346 | Ches1 | 2287 | 3095 |
| NM_008796 | Pctp | 2288 | 3096 |
| AY004874 | Slc23a1 | 2289 | 3097 |
| AK009258 | 2310009O17Rik | 2290 | 3098 |
| AK002859 | Aspa | 2291 | 3099 |
| BB483938 | AI452195 | 2292 | 3100 |
| AK013679 | 2900053I11Rik | 2293 | 3101 |
| AK017598 | 5730422A13Rik | 2294 | 3102 |
| AK010891 | 2510002J07Rik | 22 | 72 |
| NM_010431 | Hif1a | 2295 | 3103 |
| | | | 3104 |
| AK002480 | 0610010I13Rik | 1574 | 1833 |
| AK009374 | 2310016A09Rik | 912 | 966 |
| AK006771 | 1700052K11Rik | 2296 | 3105 |
| AK016911 | 4933425E08Rik | 2297 | 3106 |
| NM_007635 | Ccng2 | 2298 | 3107 |
| NM_010160 | Cugbp2 | 2299 | 3108 |
| NM_022434 | Cyp4f14 | 2300 | 3109 |
| AK013725 | Dnclc1 | 2301 | 3110 |
| NM_009824 | Cbfa2t3h | 2302 | 3111 |
| AK007630 | Cdkn1a | 2303 | 3112 |
| | | | 3113 |
| AK006385 | 1700026H06Rik | 2304 | 3114 |
| AI875461 | AI875461 | 2305 | 3115 |
| AK004319 | 1110059L23Rik | 2306 | 3116 |
| BE990725 | BE990725 | 2307 | 3117 |
| NM_009362 | Tff1 | 2308 | 3118 |
| NM_011723 | Xdh | 2309 | 3119 |
| NM_010863 | Myo1b | 2310 | 3120 |
| AK004905 | 1300004O04Rik | 2311 | 3121 |
| NM_008391 | Irf2 | 2312 | 3122 |
| AK014490 | 3110020O18Rik | 2313 | 3123 |
| AK017615 | Sec61a2-pending | 2314 | 3124 |
| AK009820 | 2310045I24Rik | 2315 | 3125 |
| BB358694 | LOC217698 | 2316 | 3126 |
| AK002528 | Cyp4a10 | 2317 | 3127 |
| BB234992 | LOC217698 | 2318 | 3128 |
| AK010202 | 2310076L09Rik | 2319 | 3129 |
| AK018164 | 6330412C24Rik | 2320 | 3130 |
| AK005010 | 1300015B04Rik | 2321 | 3131 |
| NM_026164 | 1200006O19Rik | 2322 | 3132 |
| AK005064 | 1300019I21Rik | 2323 | 3133 |
| NM_008645 | Mug1 | 2324 | 3134 |
| NM_016915 | Pla2g6 | 2325 | 3135 |
| NM_030565 | BC004044 | 2326 | 3136 |
| NM_010255 | Gamt | 2327 | 3137 |
| NM_008555 | Masp1 | 2328 | 3138 |
| BB498227 | BB498227 | 2329 | 3139 |
| AK011462 | 2610019F03Rik | 2330 | 3140 |
| BB160481 | BB160481 | 2331 | 3141 |
| AK018558 | 9030618K22Rik | 2332 | 3142 |
| AK009057 | 2310001A20Rik | 2333 | 3143 |
| AK009156 | 2310004N24Rik | 2334 | 3144 |
| AF377871 | Pawr | 2335 | 3145 |
| AK005014 | 1300015D01Rik | 2336 | 3146 |
| NM_025621 | 2310050C09Rik | 2337 | 3147 |
| NM_025459 | 1810015C04Rik | 2338 | 3148 |
| AK009724 | 2310040G24Rik | 2339 | 3149 |
| BE993937 | AI666798 | 2340 | 3150 |
| X70514 | Nodal | 2341 | 3151 |
| AK020074 | 6030458C11Rik | 2342 | 3152 |
| AK005383 | Pcbp4 | 2343 | 3153 |
| AK016973 | 4833415F11Rik | 2344 | 3154 |
| NM_007865 | DII1 | 2345 | 3155 |
| AK009083 | Gale | 2346 | 3156 |
| AK012415 | 2700053F16Rik | 2347 | 3157 |
| NM_013534 | Grcb | 2348 | 3158 |
| AV294988 | Tacc2 | 2349 | 3159 |
| AK010289 | 2400006N03Rik | 2350 | 3160 |
| AK015259 | 493043l09Rik | 2351 | 3161 |
| AK013911 | Igsf4 | 2352 | 3162 |
| BB157693 | BB157693 | 2353 | 3163 |
| BF018327 | H2-M10.1 | 2354 | 3164 |
| AK011266 | Gdm1 | 2355 | 3165 |
| NM_024240 | 4933405K01Rik | 2356 | 3166 |
| AK008690 | Abhd2 | 2357 | 3167 |
| NM_008156 | Gpld1 | 2358 | 3168 |
| AK006091 | 1700018L02Rik | 2359 | 3169 |
| AK007264 | 1700124F02Rik | 2360 | 3170 |
| AK021282 | AI848120 | 2361 | 3171 |
| AK008072 | 2010003K11Rik | 2362 | 3172 |
| NM_007954 | Es1 | 2363 | 3173 |
| AK017446 | 5530402H23Rik | 2364 | 3174 |
| NM_023207 | W1d | 2365 | 3175 |
| BC002253 | AI314967 | 2366 | 3176 |
| NM_008223 | Serpind1 | 2367 | 3177 |
| AK009154 | 2310004N11Rik | 2368 | 3178 |
| AK009435 | D17Wsu51e | 2369 | 3179 |
| AK004708 | 1200011I23Rik | 2370 | 3180 |
| NM_021371 | Caln1 | 2371 | 3181 |
| AK005346 | 1500032M05Rik | 2372 | 3182 |
| NM_019687 | Slc22a4 | 2373 | 3183 |
| AK008038 | Slc25a10 | 2374 | 3184 |
| AK004692 | Sdh1 | 2375 | 3185 |
| NM_019867 | Ngef | 2376 | 3186 |
| AK007649 | 1810030A06Rik | 2377 | 3187 |
| NM_010321 | Gnmt | 2378 | 3188 |
| AK010239 | Fzd7 | 2379 | 3189 |
| AK008081 | D15Ertd747e | 2380 | 3190 |
| AK007644 | Dexi | 2381 | 3191 |
| AK012103 | Hsd17b12 | 2382 | 3192 |
| AK014853 | 4921509J17Rik | 2383 | 3193 |
| AK010372 | 2410003M15Rik | 2384 | 3194 |
| NM_011172 | Prodh | 2385 | 3195 |
| AK018414 | 8430415E04Rik | 2386 | 3196 |
| AK015901 | MGC28623 | 2387 | 3197 |
| BC003470 | Pspla1-pending | 2388 | 3198 |
| NM_009040 | Rdh6 | 2389 | 3199 |
| NM_007972 | F10 | 2390 | 3200 |
| AK009002 | 2300002C06Rik | 2391 | 3201 |
| AK005015 | Csad | 2392 | 3202 |
| AK007603 | 1810026B04Rik | 2393 | 3203 |
| AK008844 | 2210407G14Rik | 2394 | 3204 |
| NM_008295 | Hsd3b5 | 2395 | 3205 |
| AK021253 | C430046K18Rik | 2396 | 3206 |
| AK009918 | Cdk3 | 2397 | 3207 |
| AK002327 | 2310075M17Rik | 2398 | 3208 |
| NM_010169 | F2r | 2399 | 3209 |
| AW319694 | Bucs1 | 2400 | 3210 |
| AK014861 | 4921510J17Rik | 2401 | 3211 |
| NM_008804 | Pde9a | 2402 | 3212 |
| NM_018868 | Nol5 | 2403 | 3213 |
| BB233906 | LOC217698 | 2404 | 3214 |
| AK003407 | 1110004C05Rik | 2405 | 3215 |
| BC003974 | 4933436C10Rik | 2406 | 3216 |
| AJ272272 | Psma1 | 2407 | 3217 |
| AK014460 | 3930402G23Rik | 2408 | 3218 |
| NM_009025 | Rasa3 | 2409 | 3219 |
| AK004971 | 1300012D20Rik | 2410 | 3220 |
| AK003561 | 1110008B24Rik | 2411 | 3221 |
| AK020191 | 8030402F09Rik | 2412 | 3222 |
| AK016678 | 4933405P16Rik | 2413 | 3223 |
| NM_008655 | Gadd45b | 2414 | 3224 |
| AK017918 | 5830411H19Rik | 1614 | 1873 |
| AK005080 | Suclg1 | 916 | 970 |
| NM_021314 | Tacc2 | 2415 | 3225 |
| BB483548 | ri\|C030045D06\| | 2416 | 3226 |
| | PX00075C24\|\|1567 | | |
| NM_030692 | Sacm1l | 2417 | 3227 |
| NM_008086 | Gas1 | 2418 | 3228 |
| AK019250 | 2810030D12Rik | 2419 | 3229 |
| AK002889 | 0610041L09Rik | 917 | 971 |
| BC005585 | LOC231086 | 918 | 972 |
| AK008206 | Snrk | 2420 | 3230 |
| NM_018795 | Abcc6 | 2421 | 3231 |
| NM_025626 | 3110001A13Rik | 1620 | 1879 |
| NM_025834 | 1300015B06Rik | 2422 | 3232 |
| AK004936 | Apoa5 | 2423 | 3233 |
| NM_011068 | Pex11a | 2424 | 3234 |
| AK018684 | Hao3 | 2425 | 3235 |
| AK017563 | 5730415C11Rik | 2426 | 3236 |
| AK009450 | 2310021M12Rik | 2427 | 3237 |
| AK006541 | Fac15 | 2428 | 3238 |
| NM_020520 | Slc25a20 | 919 | 973 |
| NM_010172 | F7 | 2429 | 3239 |
| AK007384 | Sult1c1 | 2430 | 3240 |
| AK008800 | 2210402C18Rik | 2431 | 3241 |
| AK010648 | 2410041F14Rik | 2432 | 3242 |
| AK004920 | 1300006O23Rik | 2433 | 3243 |
| AK013742 | Sca10 | 2434 | 3244 |
| AK010922 | 2510006M18Rik | 2435 | 3245 |
| AK003249 | Ppp1r14a | 2436 | 3246 |
| AK016667 | 4933405K01Rik | 2437 | 3247 |
| AF307987 | Ccl21c | 2438 | 3248 |
| AK013918 | 3100002J04Rik | 2439 | 3249 |
| AK002436 | Ran | 2440 | 3250 |
| AK005003 | 1300014I06Rik | 2441 | 3251 |
| AK009263 | 2410001H17Rik | 2442 | 3252 |
| AK007239 | Meig1 | 2443 | 3253 |
| AK009310 | Fetub | 2444 | 3254 |
| AK004787 | 1200015G06Rik | 2445 | 3255 |
| AK003046 | Nrn1 | 2446 | 3256 |
| AK018565 | 9030622O22Rik | 2447 | 3257 |
| NM_010702 | Lect2 | 2448 | 3258 |
| NM_008222 | Hccs | 2449 | 3259 |
| AK015368 | 4930443B20Rik | 2450 | 3260 |
| AK021146 | C030044E10Rik | 2451 | 3261 |
| NM_016843 | Sca10 | 2452 | 3262 |
| AK004540 | Arsa | 2453 | 3263 |
| NM_033037 | Cdo1 | 2454 | 3264 |
| AV252417 | AV252417 | 2455 | 3265 |
| AK013296 | Apex1 | 2456 | 3266 |
| AW476218 | AW476218 | 2457 | 3267 |
| NM_030687 | Slc21a5 | 2458 | 3268 |
| BB533722 | BB533722 | 2459 | 3269 |
| NM_019961 | Pex3 | 1030 | 1046 |
| NM_016763 | Hsdl7b10 | 2460 | 3270 |
| NM_008777 | Pah | 2461 | 3271 |
| BF459334 | BF459334 | 2462 | 3272 |
| AK018358 | 6820402I19Rik | 2463 | 3273 |
| AK010168 | 2010004E11Rik | 2464 | 3274 |
| AK011123 | Scarb2 | 2465 | 3275 |
| BB280678 | BB280678 | 2466 | 3276 |
| NM_026178 | Mmd | 2467 | 3277 |
| NM_012057 | Irf5 | 2468 | 3278 |
| NM_010476 | Hsd17b7 | 2469 | 3279 |
| NM_009862 | Cdc451 | 2470 | 3280 |
| NM_009266 | Sps2 | 2471 | 3281 |
| NM_026011 | 2610313E07Rik | 2472 | 3282 |
| NM_026494 | AI413471 | 1031 | 1047 |
| NM_009075 | Rpia | 2473 | 3283 |
| BB540470 | Cyp4a12 | 2474 | 3284 |
| BB487754 | AI197264 | 2475 | 3285 |
| BE991963 | Enc1 | 2476 | 3286 |
| BC005792 | Pte1 | 922 | 976 |
| AK014609 | 4633401B06Rik | 2477 | 3287 |
| AK020260 | 9030421L11Rik | 2478 | 3288 |
| NM_010422 | Hexb | 1639 | 1898 |
| AK013557 | 2900019G14Rik | 2479 | 3289 |
| AK004798 | 1200015P04Rik | 2480 | 3290 |
| AB042027 | GRSP1 | 2481 | 3291 |
| AK012897 | Hbb-y | 2482 | 3292 |
| BI556028 | ri\|E130107N23\| | 2483 | 3293 |
| | PX00091H11\|\|1437 | | |
| AK014530 | 4933402G07Rik | 2484 | 3294 |
| AK014514 | 4631408O11Rik | 2485 | 3295 |
| AI450589 | 0610012F22Rik | 2486 | 3296 |
| NM_008304 | Sdc2 | 2487 | 3297 |
| AW049168 | Dscrll1 | 2488 | 3298 |
| AK018100 | 6230429P13Rik | 2489 | 3299 |
| AK011 002 | Map2k3 | 2490 | 3300 |
| AK007964 | MGC28885 | 2491 | 3301 |
| BC005529 | Rin2 | 2492 | 3302 |
| NM_008294 | Hsd3b4 | 2493 | 3303 |
| | | | 3304 |
| | | | 3305 |
| AV287497 | Xnp | 2494 | 3306 |
| AK012712 | 2810011L15Rik | 2495 | 3307 |
| BF785788 | R74766 | 2496 | 3308 |
| AK017688 | 5730469M10Rik | 2497 | 3309 |
| AK007400 | Lbh-pending | 2498 | 3310 |
| BB282142 | BB282142 | 2499 | 3311 |
| NM_011704 | Vnn1 | 2500 | 3312 |
| | | | 3313 |
| | | | 3314 |
| NM_013465 | Ahsg | 2501 | 3315 |
| NM_015755 | Hunk | 2502 | 3316 |
| BC002120 | 1810013P09Rik | 2503 | 3317 |
| NM_023617 | 1200011D03Rik | 2504 | 3318 |
| BC003451 | LOC232087 | 2505 | 3319 |
| AK007392 | Ela1 | 2506 | 3320 |
| AK016659 | 4933405A16Rik | 1645 | 1904 |
| AK020614 | 9530058B02Rik | 2507 | 3321 |
| AK021029 | B830003A16Rik | 2508 | 3322 |
| AK010119 | Ptp1a | 2509 | 3323 |
| AK003844 | 1110020B03Rik | 2510 | 3324 |
| NM_013797 | Slc21a1 | 2511 | 3325 |
| NM_016723 | Uch13 | 2512 | 3326 |
| BG961761 | ri\|9430029L20\| | 2513 | 3327 |
| | PX00109E05\|\|1326 | | |
| NM_010591 | Jun | 2514 | 3328 |
| | | | 3329 |
| | | | 3330 |
| AK012213 | Aldh1b1 | 2515 | 3331 |
| NM_025964 | 2310038H17Rik | 2516 | 3332 |
| AK002826 | 0610039C21Rik | 2517 | 3333 |
| | | | 3334 |
| AK004897 | Facl2 | 30 | 80 |
| NM_011994 | Abcd2 | 2518 | 3335 |
| AK017296 | Ntn3 | 2519 | 3336 |
| NM_016928 | Tlr5 | 2520 | 3337 |
| NM_010776 | Mbl2 | 2521 | 3338 |
| NM_012006 | Cte1 | 2522 | 3339 |
| | | | 3340 |
| | | | 3341 |
| AK002968 | 0710001L09Rik | 2523 | 3342 |
| AK007645 | Gcst | 2524 | 3343 |
| AK012581 | 0610025L06Rik | 2525 | 3344 |
| AK008702 | 2210010N10Rik | 2526 | 3345 |
| BI329624 | ri\|9530008L14\| | 2527 | 3346 |
| | PX00111H18\|\|1536 | | |
| NM_025768 | Grtp1 | 2528 | 3347 |
| NM_009624 | Adcy9 | 2529 | 3348 |
| NM_024223 | Crip2 | 1651 | 1910 |
| NM_011966 | Psma4 | 2530 | 3349 |
| AK005897 | 1700012D01Rik | 2531 | 3350 |
| NM_016748 | Ctps | 2532 | 3351 |
| AK017309 | Pex1 | 2533 | 3352 |
| AK003554 | 0610008K04Rik | 2534 | 3353 |
| NM_012050 | Omd | 2535 | 3354 |
| AK004609 | 1200006F02Rik | 2536 | 3355 |
| AK007115 | 1700102P08Rik | 2537 | 3356 |
| NM_013631 | Pklr | 2538 | 3357 |
| BB503671 | Hsd3b2 | 2539 | 3358 |
| AK019762 | 4930552P12Rik | 2540 | 3359 |
| AK019519 | 4833432B22Rik | 2541 | 3360 |
| NM_008990 | Pvrl2 | 2542 | 3361 |
| BB348963 | BB348963 | 2543 | 3362 |
| AK005546 | 1600027G01Rik | 2544 | 3363 |
| AK007970 | Acf-pending | 2545 | 3364 |
| AK003859 | Rtn4 | 2546 | 3365 |
| | | | 3366 |
| | | | 3367 |
| AK017475 | 5730402C02Rik | 2547 | 3368 |
| NM_023175 | D16Ertd502e | 2548 | 3369 |
| AK018142 | 6330408G06Rik | 2549 | 3370 |
| AK008100 | 2010004M01Rik | 2550 | 3371 |
| AK002565 | Ap3s1 | 2551 | 3372 |
| AK003760 | 1110017O10Rik | 2552 | 3373 |
| BB166389 | 5730408C10Rik | 2553 | 3374 |
| AK004889 | Acadsb | 2554 | 3375 |
| BC002130 | Dusp14 | 2555 | 3376 |
| NM_023792 | Pank | 2556 | 3377 |
| BC003479 | LOC216820 | 35 | 86 |
| AK003397 | 1110003P22Rik | 2557 | 3378 |
| AK019381 | Pxmp4 | 2558 | 3379 |
| NM_007686 | Cfi | 2559 | 3380 |
| NM_007976 | F5 | 2560 | 3381 |
| NM_011375 | Siat9 | 2561 | 3382 |
| AK018506 | 8430438D04Rik | 2562 | 3383 |
| AF102849 | Haik1-pending | 2563 | 3384 |
| AK008673 | 2210008K22Rik | 2564 | 3385 |
| NM_011792 | Bace | 2565 | 3386 |
| NM_022882 | Lpin2 | 2566 | 3387 |
| AK015721 | 4930506M07Rik | 2567 | 3388 |
| NM_019933 | Ptpn4 | 2568 | 3389 |
| AK011880 | 2610204K03Rik | 2569 | 3390 |
| NM_018884 | Semcap3-pending | 2570 | 3391 |
| AK016577 | 4932702F08Rik | 2571 | 3392 |
| AK018332 | 6530411B15Rik | 2572 | 3393 |
| AK017185 | 5033421K01Rik | 2573 | 3394 |
| NM_011937 | Gnpi | 2574 | 3395 |
| AK019527 | Wrnip | 2575 | 3396 |
| NM_010062 | Dnase2a | 2576 | 3397 |
| AW494273 | AW494273 | 2577 | 3398 |
| AK008793 | 2210401N16Rik | 2578 | 3399 |
| NM_010158 | Khdrbs3 | 2579 | 3400 |
| NM_013565 | Itga3 | 2580 | 3401 |
| AK009895 | Sfrs3 | 2581 | 3402 |
| NM_025994 | 2600015J22Rik | 2582 | 3403 |
| NM_025341 | 0610041D24Rik | 2583 | 3404 |
| AK013477 | 1110011E12Rik | 2584 | 3405 |
| AK010387 | 2410004H02Rik | 2585 | 3406 |
| AK011735 | Ppp2r4 | 2586 | 3407 |
| NM_007799 | Ctse | 2587 | 3408 |
| NM_016689 | Aqp3 | 2588 | 3409 |
| AK006350 | Rasl2-9 | 2589 | 3410 |
| AK008555 | Pso | 2590 | 3411 |
| AF177211 | Gpr105 | 2591 | 3412 |
| AK014427 | 3830408G10Rik | 2592 | 3413 |
| NM_008574 | Mcsp | 2593 | 3414 |
| NM_016917 | Slc39a1 | 2594 | 3415 |
| NM_016918 | Nudt5 | 2595 | 3416 |
| AB055897 | AW413091 | 2596 | 3417 |
| AK017223 | 5133401H06Rik | 2597 | 3418 |
| NM_013697 | Ttr | 2598 | 3419 |
| AK003996 | 1110030O19Rik | 2599 | 3420 |
| AK003495 | 1110006G02Rik | 2600 | 3421 |
| AK020110 | Lbh-pending | 2601 | 3422 |
| AK015173 | 4930421P07Rik | 2602 | 3423 |
| AK014774 | 4833426J09Rik | 2603 | 3424 |
| NM_013792 | Nag1u | 1673 | 1932 |
| NM_008455 | Klkb1 | 2604 | 3425 |
| NM_019840 | Pde4b | 2605 | 3426 |
| NM_011920 | Abcg2 | 2606 | 3427 |
| AK020473 | 9430063L05Rik | 2607 | 3428 |
| AC002397 | CD4, A-2, B, GNB3, | 1674 | 1933 |
| | C8, ISOT, TPI, B7, | | |
| | ENO2, DRPLA, | | |
| | U7snRNA, C10, PTPN6, | | |
| | BAP,C2F | | |
| NM_019878 | Sult1b1 | 2608 | 3429 |
| NM_022014 | Fn3k | 2609 | 3430 |
| BC002197 | C79952 | 2610 | 3431 |
| AK002691 | D14Uc1a2 | 2611 | 3432 |
| NM_019877 | Copz2 | 2612 | 3433 |
| AK017527 | 5730408K05Rik | 2613 | 3434 |
| AK016217 | 4930564C03Rik | 2614 | 3435 |
| AK008119 | 2010005E21Rik | 2615 | 3436 |
| NM_019983 | Rab5ef-pending | 2616 | 3437 |
| NM_025597 | 2700033I16Rik | 2617 | 3438 |
| AK013580 | 2900024C23Rik | 1682 | 1941 |
| NM_008063 | G6pt1 | 2618 | 3439 |
| AK002609 | 0610012J09Rik | 2619 | 3440 |
| BC003725 | BC003725 | 2620 | 3441 |
| AK020692 | Dbi | 2621 | 3442 |
| AK002641 | 0610016O18Rik | 2622 | 3443 |
| AB042745 | Nox4 | 2623 | 3444 |
| BE988332 | BE988332 | 2624 | 3445 |
| AK008235 | 2010013I23Rik | 2625 | 3446 |
| NM_009900 | Clcn2 | 2626 | 3447 |
| NM_008639 | Mtnr1a | 2627 | 3448 |
| AK020546 | 9530006C21Rik | 2628 | 3449 |
| AK008532 | 2610318G18Rik | 2629 | 3450 |
| AK009250 | 2310009E07Rik | 2630 | 3451 |
| AK010068 | D8Ertd91e | 2631 | 3452 |
| AK013269 | 2810439K08Rik | 2632 | 3453 |
| AK002408 | 0610009I22Rik | 2633 | 3454 |
| AK019969 | 5730504C04Rik | 2634 | 3455 |
| NM_027853 | 0610006F02Rik | 2635 | 3456 |
| BC003306 | Def8 | 2636 | 3457 |
| NM_010501 | Ifit3 | 2637 | 3458 |
| NM_007494 | Ass1 | 2638 | 3459 |
| AK008954 | 2210416J07Rik | 2639 | 3460 |
| AV059994 | AV059994 | 2640 | 3461 |
| AK010810 | 2410150I18Rik | 2641 | 3462 |
| NM_009196 | Slc16a1 | 2642 | 3463 |
| BF682011 | Ugp2 | 2643 | 3464 |
| AI195543 | MGC29978 | 1033 | 1049 |
| BE993080 | Hsd17b11 | 2644 | 3465 |
| M16357 | Mup3 | 2645 | 3466 |
| M14044 | Anxa2 | 2646 | 3467 |
| Y10221 | Cyp4a12 | 2647 | 3468 |
| AA239277 | Crot | 2648 | 3469 |
| X01756 | Cycs | 934 | 989 |
| BC007172 | Galnt2 | 2649 | 3470 |
| L02331 | Sult1a1 | 1694 | 1953 |
| M17818 | Mup1 | 2650 | 3471 |
| NM_009360 | Tfam | 2651 | 3472 |
| | | | 3473 |
| BE947329 | AW109744 | 2652 | 3474 |
| AF009605 | Pck1 | 2653 | 3475 |
| | | | 3476 |
| M21285 | Scd1 | 2654 | 3477 |
| | | | 3478 |
| X53451 | Gstp2 | 2655 | 3479 |
| X71479 | Cyp4a12 | 2656 | 3480 |
| | | | 3481 |
| | | | 3482 |
| BF449960 | AW554572 | 2657 | 3483 |
| NM_008615 | Mod1 | 2658 | 3484 |
| | | | 3485 |
| | | | 3486 |
| W50759 | Apoc3 | 2659 | 3487 |
| AI648018 | 2610207I16Rik | 936 | 991 |
| | | | 992 |
| | | | 993 |
| M10022 | Cyp1a2 | 2660 | 3488 |
| | | | 3489 |
| | | | 3490 |
| U57999 | Psap | 2661 | 3491 |
| Z14050 | Dci | 1034 | 1050 |
| W54127 | Acat1 | 2662 | 3492 |
| | | | 3493 |
| | | | 3494 |
| Y09085 | Hif1a | 2663 | 3495 |
| AI155095 | AI155095 | 2664 | 3496 |
| X51397 | Myd88 | 2665 | 3497 |
| | | | 3498 |
| Y11638 | Cyp4a14 | 2666 | 3499 |
| | | | 3500 |
| | | | 3501 |
| L33417 | V1d1r | 2667 | 3502 |
| AW909415 | 1110048B16Rik | 2668 | 3503 |
| AJ007749 | Casp8 | 2669 | 3504 |
| AJ131522 | Mlycd | 937 | 1051 |
| | | | 994 |
| AJ011967 | Gdf15 | 2670 | 3505 |
| M64248 | Apoa4 | 2671 | 3506 |
| M30697 | Abcb1a | 2672 | 3507 |
| AB010826 | Cpt1b | 2673 | 3508 |
| | | | 3509 |
| | | | 3510 |
| NM_008342 | Igfbp2 | 2674 | 3511 |
| | | | 3512 |
| | | | 3513 |
| AW986355 | Aco2 | 2675 | 3514 |
| AW456981 | Mg11 | 2676 | 3515 |
| NM_025670 | 5730403B10Rik | 2677 | 3516 |
| X00945 | Spi1-6 | 2678 | 3517 |
| X06454 | C4 | 2679 | 3518 |
| AF072757 | Slc27a2 | 2680 | 3519 |
| | | | 3520 |
| | | | 3521 |
| M25944 | Car2 | 2681 | 3522 |
| M13264 | Fabp4 | 210 | 215 |
| | | | 216 |
| | | | 3523 |
| D16215 | Fmo1 | 2682 | 3524 |
| AF064088 | Tieg | 2683 | 3525 |
| NM_013743 | Pdk4 | 42 | 93 |
| | | | 998 |
| | | | 94 |
| BC008241 | Psmb4 | 2684 | 3526 |
| Z71189 | Acadv1 | 939 | 1001 |
| | | | 999 |
| | | | 1000 |
| S75207 | Hsd11b1 | 2685 | 3527 |
| | | | 3528 |
| | | | 3529 |
| AB033885 | Fac14 | 2686 | 3530 |
| | | | 3531 |
| | | | 3532 |
| AA591552 | Hsp86-1 | 2687 | 3533 |
| AA986766 | AA986766 | 2688 | 3534 |
| AB003303 | Slc10a1 | 2689 | 3535 |
| AB006361 | Ptgds | 2690 | 3536 |
| AF006688 | Acox1 | 1444 | 1465 |
| | | | 1467 |
| | | | 1466 |
| AF007267 | Pmm1 | 1698 | 1957 |
| AF030343 | Ech1 | 940 | 1002 |
| AF031814 | Nr1i2 | 2691 | 3537 |
| AF033196 | Rdh5 | 2692 | 3538 |
| AF038939 | Peg3 | 2693 | 3539 |
| AJ001118 | Mg11 | 209 | 214 |
| D17674 | Cyp2c29 | 2694 | 3540 |
| | | | 3541 |
| | | | 3542 |
| D28530 | Ptprs | 2695 | 3543 |
| D29016 | Fdft1 | 2696 | 3544 |
| | | | 3545 |
| | | | 3546 |
| D86563 | Rab4a | 2697 | 3547 |
| J03398 | Abcb4 | 2698 | 3548 |
| | | | 3549 |
| | | | 3550 |
| J03549 | Cyp2a4 | 2699 | 3551 |
| | | | 3552 |
| | | | 3553 |
| J04696 | Gstm2 | 1703 | 1962 |
| L20509 | Cct3 | 2700 | 3554 |
| L31783 | Umpk | 2701 | 3555 |
| L47970 | Mttp | 2702 | 3556 |
| | | | 3557 |
| | | | 3558 |
| M16465 | S100a10 | 2703 | 3559 |
| M21065 | Irf1 | 2704 | 3560 |
| M21856 | Cyp2b10 | 2705 | 3561 |
| M27167 | Cyp2d10 | 2706 | 3562 |
| M29008 | AI194696 | 2707 | 3563 |
| M29009 | Cfh | 2708 | 3564 |
| M31885 | Idb1 | 2709 | 3565 |
| M64250 | Apoa4 | 2710 | 3566 |
| | | | 3567 |
| | | | 3568 |
| M75886 | Hsd3b2 | 2711 | 3569 |
| M77003 | Gpam | 2712 | 3570 |
| | | | 3571 |
| | | | 3572 |
| M77497 | Cyp2f2 | 2713 | 3573 |
| M83649 | Tnfrsf6 | 2714 | 3574 |
| M93275 | Adfp | 943 | 1007 |
| | | | 1005 |
| | | | 1006 |
| U01163 | Cpt2 | 1035 | 1056 |
| | | | 1057 |
| U07159 | Acadm | 945 | 1009 |
| | | | 1011 |
| | | | 1010 |
| U09507 | Cdkn1a | 2715 | 3575 |
| | | | 3576 |
| U13371 | Kdt1 | 2716 | 3577 |
| U14332 | Il15 | 1710 | 1974 |
| U21489 | Acad1 | 946 | 1014 |
| | | | 1012 |
| | | | 1013 |
| U23922 | Il12rb1 | 2717 | 3578 |
| U36993 | Cyp7b1 | 2718 | 3579 |
| | | | 3580 |
| | | | 3581 |
| U38196 | Mpp1 | 2719 | 3582 |
| U43298 | Lamb3 | 1711 | 1975 |
| U47543 | Nab2 | 2720 | 3583 |
| U48403 | Gyk | 2721 | 3584 |
| | | | 3585 |
| | | | 3586 |
| U48420 | Gstt2 | 2722 | 3587 |
| U58883 | Sorbs1 | 1712 | 1977 |
| | | | 3588 |
| U59418 | Ppp2r5c | 2723 | 3589 |
| U60987 | Gdm1 | 2724 | 3590 |
| | | | 3591 |
| U79550 | Snai2 | 1714 | 1979 |
| U83176 | Gt(ROSA)26asSor | 2725 | 3592 |
| U89491 | Ephx1 | 2726 | 3593 |
| X04480 | Igf1 | 2727 | 3594 |
| X05475 | C9 | 2728 | 3595 |
| X13135 | Fasn | 2729 | 3596 |
| | | | 3597 |
| | | | 3598 |
| X53584 | Hsp60 | 2730 | 3599 |
| X62940 | Tgfb1i4 | 2731 | 3600 |
| X70067 | Rnps1 | 2732 | 3601 |
| X70398 | D0H4S114 | 948 | 1016 |
| X83971 | Fos12 | 2733 | 3602 |
| X89864 | Cyp2a5 | 2734 | 3603 |
| | | | 3551 |
| | | | 3604 |
| X89998 | Hsdl7b4 | 949 | 1018 |
| | | | 1017 |
| | | | 1019 |
| X96618 | Rga | 2735 | 3605 |
| Y14660 | Fabp1 | 2736 | 3606 |
| | | | 3607 |
| | | | 3608 |
| D87521 | Prkdc | 2737 | 3609 |
| M33960 | Serpine1 | 2738 | 3610 |
| | | | 3611 |
| | | | 3612 |
| AF071315 | Cops6 | 2739 | 3613 |
| U33557 | Fpgs | 2740 | 3614 |
| X95280 | G0s2 | 2741 | 3615 |
| ABO11000 | Chk1 | 2742 | 3616 |
| AF026073 | Sultn | 2743 | 3617 |
| AJ000059 | Hyal2 | 2744 | 3618 |
| M14757 | Abcb1b | 2745 | 3619 |
| M61737 | Fsp27 | 2746 | 3620 |
| AF075717 | TIF2 | 2747 | 3621 |
| AI326224 | AI326224 | 2748 | 3622 |
| J00423 | Hprt | 2749 | 3623 |
| | | | 3624 |
| | | | 3625 |
| L23108 | Cd36 | 142 | 3626 |
| | | | 3627 |
| | | | 3628 |
| X00479 | Cyp1a2 | 2750 | 3488 |
| | | | 3489 |
| | | | 3490 |
| AI118433 | C8a | 2751 | 3629 |
| AI132306 | AI132306 | 2752 | 3630 |
| AI255955 | Il1rap | 2753 | 3631 |
| AI265707 | AI265623 | 2754 | 3632 |
| AI663818 | AI663818 | 2755 | 3633 |
| AI854637 | | 2756 | 3634 |
| AI132665 | LOC208677 | 2757 | 3635 |
| AI255958 | LOC226105 | 2758 | 3636 |
| AI266885 | AI266885 | 2759 | 3637 |
| AI530213 | Ugp2 | 2760 | 3638 |
| AI461749 | AI451155 | 2761 | 3639 |
| AI464465 | | 2762 | 3640 |
| AI503986 | | 2763 | 3641 |
| D16333 | Cpo | 2764 | 3642 |
| X78683 | Bcap37 | 2765 | 3643 |
| AI482473 | Syt14 | 2766 | 3644 |
| AI662255 | AI662255 | 2767 | 3645 |
| AI785285 | Dscr111 | 2768 | 3646 |
| AI851538 | Kcnn2 | 2769 | 3647 |
| AB027290 | Rab9 | 2770 | 3648 |
| AF126798 | Fads2 | 2771 | 3649 |
| | | | 3650 |
| | | | 3651 |
| NM_011080 | Phxr1 | 2772 | 3652 |
| U12790 | Hmgcs2 | 2773 | 3653 |
| | | | 3654 |
| | | | 3655 |
| NM_008686 | Nfe211 | 2774 | 3656 |
| AB017136 | Homer2-pending | 2775 | 3657 |
| NM_007843 | Defb1 | 2776 | 3658 |
| AI647584 | AI647584 | 2777 | 3659 |
| AW060343 | AW060343 | 2778 | 3660 |
| AI647917 | 3200002M13Rik | 2779 | 3661 |
| AI595938 | AI595938 | 2780 | 3662 |
| NM_010284 | Ghr | 1728 | 1994 |
| AW061234 | AW061234 | 2781 | 3663 |
| NM_008509 | Lp1 | 2782 | 3664 |
| | | | 3665 |
| | | | 3666 |
| Z37107 | Ephx2 | 49 | 101 |
| AI324870 | AI324870 | 2783 | 3667 |
| X84014 | Lama3 | 2784 | 3668 |
| Z31362 | Npn3 | 2785 | 3669 |
| U39066 | Map2k6 | 2786 | 3670 |
| Z97207 | Hspc121-pending | 2787 | 3671 |
| AF161071 | Slc2a5 | 2788 | 3672 |
| | | | 3673 |
| AI646798 | AI646798 | 2789 | 3674 |
| AF133903 | Abcb11 | 2790 | 3675 |
| | | | 3676 |
| NM_008254 | Hmgc1 | 2791 | 3677 |
| | | | 3678 |
| | | | 3679 |
| AF112185 | Scnn1a | 2792 | 3680 |
| AI642194 | AI463690 | 2793 | 3681 |
| AI893641 | AI893641 | 2794 | 3682 |
| AI596436 | AI596436 | 2795 | 3683 |

While the preferred embodiment of the invention has been illustrated and described, it will be appreciated that various changes can be made therein without departing from the spirit and scope of the invention.

1. A method for determining whether an agent possesses a defined biological activity, the method comprising the steps of:
(a) making at least one comparison from the group consisting of:
(1) comparing an efficacy value of the agent to at least one reference efficacy value to yield an efficacy comparison result, wherein each efficacy value represents at least one expression pattern of the same efficacy-related population of genes, or at least one expression pattern of the same efficacy-related population of proteins;
(2) comparing a toxicity value of the agent to at least one reference toxicity value to yield a toxicity comparison result, wherein each toxicity value represents at least one expression pattern of the same toxicity-related population of genes, or at least one expression pattern of the same toxicity-related population of proteins;
(3) comparing a classifier value of the agent to at least one reference classifier value to yield a classifier comparison result, wherein each classifier value represents at least one expression pattern of the same classifier population of genes, or at least one expression pattern of the same classifier population of proteins; and
(b) using the comparison result(s) obtained in step (a) to determine whether the agent possesses the defined biological activity.

2. The method of claim 1 comprising the steps of:
(a) making at least two comparisons from the group consisting of:
(1) comparing an efficacy value of the agent to at least one reference efficacy value to yield an efficacy comparison result, wherein each efficacy value represents at least one expression pattern of the same efficacy-related population of genes, or at least one expression pattern of the same efficacy-related population of proteins;
(2) comparing a toxicity value of the agent to at least one reference toxicity value to yield a toxicity comparison result, wherein each toxicity value represents at least one expression pattern of the same toxicity-related population of genes, or at least one expression pattern of the same toxicity-related population of proteins;
(3) comparing a classifier value of the agent to at least one reference classifier value to yield a classifier comparison result, wherein each classifier value represents at least one expression pattern of the same classifier population of genes, or at least one expression pattern of the same classifier population of proteins; and
(b) using the comparison results obtained in step (a) to determine whether the agent possesses the defined biological activity.

3. The method of claim 1 comprising the steps of:
(a) comparing an efficacy value of the agent to at least one reference efficacy value to yield an efficacy comparison result, wherein each efficacy value represents at least one expression pattern of the same efficacy-related population of genes, or at least one expression pattern of the same efficacy-related population of proteins;
(b) comparing a toxicity value of the agent to at least one reference toxicity value to yield a toxicity comparison result, wherein each toxicity value represents at least one expression pattern of the same toxicity-related population of genes, or at least one expression pattern of the same toxicity-related population of proteins;
(c) comparing a classifier value of the agent to at least one reference classifier value to yield a classifier comparison result, wherein each classifier value represents at least one expression pattern of the same classifier population of genes, or at least one expression pattern of the same classifier population of proteins; and
(d) using the efficacy comparison result, the toxicity comparison result and the classifier comparison result to determine whether the agent possesses the defined biological activity, wherein steps (a), (b) and (c) can occur in any order with respect to each other.

4. The method of claim 1 wherein the agent is a chemical agent.

5. The method of claim 1 wherein the defined biological activity is stimulation of a biological response.

6. The method of claim 1 wherein the defined biological activity is inhibition of a biological response.

7. The method of claim 1 wherein the defined biological activity is amelioration of at least one symptom of a disease in a mammal.

8. The method of claim 1 wherein the defined biological activity is partial agonist activity with respect to a biological response, or with respect to a protein that mediates a biological response.

9. The method of claim 8 wherein the defined biological activity is partial agonist activity with respect to PPARγ.

10. The method of claim 1 wherein the at least one reference efficacy value is the efficacy value of a reference agent that possesses the defined biological activity.

11. The method of claim 1 wherein the at least one reference toxicity value is the toxicity value of a reference agent that possesses the defined biological activity.

12. The method of claim 1 wherein the at least one reference classifier value is the classifier value of a reference agent that possesses the defined biological activity.

13. The method of claim 1 wherein at least one member of the group consisting of the efficacy value of the agent, the toxicity value of the agent and the classifier value of the agent is calculated from at least one member of the group consisting of gene expression levels and protein expression levels measured in living cells cultured in vitro.

14. The method of claim 13 wherein at least two members of the group consisting of the efficacy value of the agent, the toxicity value of the agent and the classifier value of the agent are calculated from at least one member of the group consisting of gene expression levels and protein expression levels measured in living cells cultured in vitro.

15. The method of claim 13 wherein the efficacy value of the agent, the toxicity value of the agent and the classifier value of the agent are calculated from at least one member of the group consisting of gene expression levels and protein expression levels measured in living cells cultured in vitro.

16. The method of claim 13 wherein the living cells are selected from the group consisting of heart cells, liver cells and adipocyte cells.

17. The method of claim 16 wherein the living cells are 3T3L1 adipocyte cells.

18. The method of claim 1 wherein the defined biological activity is the ability to affect a biological process in vivo, and wherein at least one member of the group consisting of the efficacy value of the agent, the toxicity value of the agent and the classifier value of the agent is calculated from at least one member of the group consisting of gene expression levels and protein expression levels measured in living cells cultured in vitro.

19. The method of claim 18 wherein the biological process is an acute or chronic disease in a mammal.

20. The method of claim 1 wherein the defined biological activity is the ability to affect a biological process in vivo, and wherein at least two members of the group consisting of the efficacy value of the agent, the toxicity value of the agent and the classifier value of the agent are calculated from at least one member of the group consisting of gene expression levels and protein expression levels measured in living cells cultured in vitro.

21. The method of claim 20 wherein the biological process is an acute or chronic disease in a mammal.

22. The method of claim 1 wherein the defined biological activity is the ability to affect a biological process in vivo, and wherein the efficacy value of the agent, the toxicity value of the agent and the classifier value of the agent are calculated from at least one member of the group consisting of gene expression levels and protein expression levels measured in living cells cultured in vitro.

23. The method of claim 22 wherein the biological process is an acute or chronic disease in a mammal.

24. The method of claim 1 wherein the defined biological activity is the ability to affect a biological process in a first living tissue, and wherein at least one member of the group consisting of the efficacy value of the agent, the toxicity value of the agent and the classifier value of the agent is calculated from at least one member of the group consisting of gene expression levels and protein expression levels measured in a second living tissue, wherein the first living tissue is a different type of tissue than the second living tissue.

25. The method of claim 1 wherein the defined biological activity is the ability to affect a biological process in a first living tissue, and wherein at least two members of the group consisting of the efficacy value of the agent, the toxicity value of the agent and the classifier value of the agent are calculated from at least one member of the group consisting of gene expression levels and protein expression levels measured in a second living tissue, wherein the first living tissue is a different type of tissue from the second living tissue.

26. The method of claim 1 wherein the defined biological activity is the ability to affect a biological process in a first living tissue, and wherein the efficacy value of the agent, the toxicity value of the agent and the classifier value of the agent are calculated from at least one member of the group consisting of gene expression levels and protein expression levels measured in a second living tissue, wherein the first living tissue is a different type of tissue than the second living tissue.

27. The method of claim 1 wherein at least one member of the group consisting of the efficacy-related population of genes and the efficacy-related population of proteins yields at least one efficacy-related gene expression pattern, or efficacy-related protein expression pattern, in response to the agent, that correlates with the presence of at least one desired biological response caused by the agent in a living thing, wherein the at least one efficacy-related gene expression pattern, or at least one efficacy-related protein expression pattern, appears before the desired biological response.

28. The method of claim 1 wherein at least one member of the group consisting of the toxicity-related population of genes and the toxicity-related population of proteins yields at least one toxicity-related gene expression pattern, or toxicity-related protein expression pattern, in response to the agent, that correlates with the presence of at least one undesirable biological response caused by the agent in a living thing, wherein the at least one toxicity-related gene expression pattern, or at least one toxicity-related protein expression pattern, appears before the undesirable biological response.

29. The method of claim 1 wherein (1) at least one member of the group consisting of the efficacy-related population of genes and the efficacy-related population of proteins yields at least one efficacy-related gene expression pattern, or efficacy-related protein expression pattern, in response to the agent, that correlates with the presence of at least one desired biological response caused by the agent in a living thing, wherein the at least one efficacy-related gene expression pattern, or at least one efficacy-related protein expression pattern, appears before the desired biological response; and (2) at least one member of the group consisting of the toxicity-related population of genes and the toxicity-related population of proteins yields at least one toxicity-related gene expression pattern, or at least one toxicity-related protein expression pattern, in response to the agent, that correlates with the presence of at least one undesirable biological response caused by the agent in a living thing, wherein the at least one toxicity-related gene expression pattern, or at least one toxicity-related protein expression pattern, appears before the undesirable biological response.

30. The method of claim 1 comprising the steps of:
(a) making at least one comparison from the group consisting of:
(1) comparing an efficacy value of the agent to a scale of efficacy values to yield an efficacy comparison result, wherein each efficacy value represents at least one expression pattern of the same efficacy-related population of genes, or at least one expression pattern of the same efficacy-related population of proteins;
(2) comparing a toxicity value of the agent to a scale of toxicity values to yield a toxicity comparison result, wherein each toxicity value represents at least one expression pattern of the same toxicity-related population of genes, or at least one expression pattern of the same toxicity-related population of proteins;
(3) comparing a classifier value of the agent to a scale of classifier values to yield a classifier comparison result, wherein each classifier value represents at least one expression pattern of the same classifier population of genes, or at least one expression pattern of the same classifier population of proteins; and
(b) using the comparison result(s) obtained in step (a) to determine whether the agent possesses the defined biological activity.

31. The method of claim 30 comprising the steps of:
(a) making at least two comparisons from the group consisting of:
(1) comparing an efficacy value of the agent to a scale of efficacy values to yield an efficacy comparison result, wherein each efficacy value represents at least one expression pattern of the same efficacy-related population of genes, or at least one expression pattern of the same efficacy-related population of proteins;
(2) comparing a toxicity value of the agent to a scale of toxicity values to yield a toxicity comparison result, wherein each toxicity value represents at least one expression pattern of the same toxicity-related population of genes, or at least one expression pattern of the same toxicity-related population of proteins;
(3) comparing a classifier value of the agent to a scale of classifier values to yield a classifier comparison result, wherein each classifier value represents at least one expression pattern of the same classifier population of genes, or at least one expression pattern of the same classifier population of proteins; and
(b) using the comparison results obtained in step (a) to determine whether the agent possesses the defined biological activity.

32. The method of claim 30 comprising the steps of:
(a) comparing an efficacy value of the agent to a scale of efficacy values to yield an efficacy comparison result, wherein each efficacy value represents at least one expression pattern of the same efficacy-related population of genes, or at least one expression pattern of the same efficacy-related population of proteins;
(b) comparing a toxicity value of the agent to a scale of toxicity values to yield a toxicity comparison result, wherein each toxicity value represents at least one expression pattern of the same toxicity-related population of genes, or at least one expression pattern of the same toxicity-related population of proteins;
(c) comparing a classifier value of the agent to a scale of classifier values to yield a classifier comparison result, wherein each classifier value represents at least one expression pattern of the same classifier population of genes, or at least one expression pattern of the same classifier population of proteins; and
(d) using the efficacy comparison result, the toxicity comparison result and the classifier comparison result to determine whether the agent possesses the defined biological activity, wherein steps (a), (b) and (c) can occur in any order with respect to each other.

33. A population of oligonucleotide probes selected from the group consisting of the population of oligonucleotide probes set forth in Table 1 (SEQ ID NOs: 51-102), the population of oligonucleotide probes set forth in Table 2 (SEQ ID NOs: 52, 53, 58, 59, 65, 66, 68, 69, 71, 73, 75, 76, 78, 82, 86, 88-90, 93, 94, 96, 101), the population of oligonucleotide probes set forth in Table 4 (SEQ ID NOs: 153-207), the population of oligonucleotide probes set forth in Table 5 (SEQ ID NOs: 213-218), the population of oligonucleotide probes set forth in Table 6 (SEQ ID NOs: 551-894, 155, 157, 164, 171, 178, 179, 185, 188, 204-206), the population of oligonucleotide probes set forth in Table 7 (SEQ ID NOs: 950-1019, 863, 93, 94, 97), the population of oligonucleotide probes set forth in Table 8 (SEQ ID NOs: 1036-1057, 951, 955, 957, 863, 959, 960, 63, 962, 966, 971-974, 980, 981, 984, 987, 989, 991-996, 93, 94, 998-1001, 97, 1004-1014, 1017-1019), the population of oligonucleotide probes set forth in Table 9 (SEQ ID NOs: 1239-1428, 558, 561, 158, 565, 574, 576, 578, 585, 592, 597, 600, 609, 612, 613, 617, 163, 625, 641-643, 646, 647, 655-657, 661, 666, 171, 681, 697, 700, 706, 707, 712, 720, 727, 740, 745, 748, 749, 755-757, 762, 766, 767, 769-771, 773, 778, 780, 786, 789, 794, 800, 803, 804, 188, 189, 191, 813, 814, 822, 823, 556, 828, 831, 832, 836, 840, 844, 864, 871, 876, 878, 883, 884, 889-891), the population of oligonucleotide probes set forth in Table 10 (SEQ ID NOs: 1449-1471, 952, 956, 957, 963, 975, 976, 981, 983, 984, 986, 990, 999-1001, 1004-1007, 1012-1014), the population of oligonucleotide probes set forth in Table 12 (SEQ ID NOs: 1731-1996, 52, 951, 1450, 957, 1452, 1455, 65, 68, 69, 72, 75, 1457, 967, 1458, 970, 971, 974, 1462, 82, 977, 978, 982, 90, 989, 990, 215, 999-1001, 96, 1468, 1005, 1006, 218, 1014, 1018, 1019), and the population of oligonucleotide probes set forth in Table 14 (SEQ ID NOs: 2796-3683, 1732, 1734, 53, 1740, 1449, 1450, 1747, 1748, 1037, 1759, 957, 1774, 60, 1780, 63, 1797, 962, 1808, 1041, 1809, 1817, 1818, 1820, 1824, 71, 72, 1833, 966, 1873, 970-973, 1879, 1046, 1047, 976, 1898, 1904, 80, 1910, 86, 1932, 1933, 1941, 1049, 989, 1953, 991-993, 1050, 1051, 994, 215, 216, 93, 94, 998-1001, 1465-1467, 1957, 1002, 214, 1962, 1005-1007, 1056, 1057, 1009-1014, 1974, 1975, 1977, 1979, 1016-1019, 1994, 101).

34. A method of identifying an efficacy-related population of genes or proteins, wherein the method comprises the steps of:
(a) contacting a living thing with an agent that is known to elicit a desired biological response; and
(b) identifying an efficacy-related population of genes or proteins in the living thing that yields an expression pattern that correlates with the occurrence of the desired biological response caused by the agent.

35. The method of claim 34 wherein the living thing is a mammal.

36. The method of claim 34 wherein the living thing is a human being.

37. The method of claim 34 wherein an efficacy-related population of genes is identified.

38. The method of claim 34 wherein an efficacy-related population of proteins is identified.

39. The method of claim 34 wherein the agent is a chemical agent.

40. The method of claim 34 wherein an efficacy-related population of genes or proteins is identified by:
(a) measuring the level of expression of each member of a multiplicity of genes or proteins in the living thing, contacted with the agent, to yield a multiplicity of expression values;
(b) measuring the level of expression of each member of the same multiplicity of genes or proteins in a reference living thing, that is not contacted with the agent, to yield a multiplicity of reference expression values; and
(c) comparing the multiplicity of expression values with the multiplicity of reference expression values to identify an efficacy-related population of genes or proteins, wherein each individual gene or protein has an expression value in response to the agent that is significantly different from the corresponding reference expression value.

41. The method of claim 34 wherein the expression pattern of the efficacy-related population of genes or proteins appears in the living thing before the occurrence of the desired biological response caused by the agent.

42. The method of claim 34 wherein the desired biological response does not occur in the living thing.

43. The method of claim 42 wherein the living thing consists essentially of epididymal white adipose tissue.

44. The method of claim 34 wherein the living thing suffers from a disease and the desired biological response is amelioration of at least one symptom of the disease.

45. The method of claim 44 wherein the living thing is a mammal, and the disease is selected from the group consisting of type II diabetes, hypercholesterolemia, cancer, inflammation, obesity, schizophrenia and Alzheimer's disease.

46. The method of claim 34 further comprising:
(a) contacting the living thing with an agent that is known to elicit at least two different desired biological responses in the living thing, wherein elicitation of a first desired biological response is mediated by a first target molecule, and elicitation of a second desired biological response is mediated by a second target molecule that is different from the first target molecule;
(b) identifying an efficacy-related population of genes or proteins that yields an expression pattern that correlates with the occurrence of the first and second desired biological responses in response to the agent;
(c) contacting a modified living thing with the agent, wherein the modified living thing is a member of the same species as the living thing and does not include any functional first target molecules;
(d) identifying an efficacy-related population of genes or proteins that yields an expression pattern that correlates with the occurrence of the second desired biological response in the modified living thing in response to the agent; and
(e) comparing the efficacy-related population of genes or proteins identified in step (b) with the efficacy-related population of genes or proteins identified in step (d) to identify an efficacy-related population of genes or proteins that yields an expression pattern that correlates with the occurrence of the first desired biological response caused by the agent.

47. The method of claim 46 wherein the first target molecule is a PPARα receptor and the second target molecule is a PPARγ receptor.

48. The method of claim 46 wherein the first target molecule is a PPARγ receptor and the second target molecule is a PPARα receptor.

49. A method of identifying a toxicity-related population of genes or proteins, wherein the method comprises the steps of:
(a) contacting a living thing with an agent that is known to elicit an undesirable biological response; and
(b) identifying a toxicity-related population of genes or proteins that yields an expression pattern that correlates with the occurrence of the undesirable biological response caused by the agent.

50. The method of claim 49 wherein the living thing is a mammal.

51. The method of claim 49 wherein the living thing is a human being.

52. The method of claim 49 wherein a toxicity-related population of genes is identified.

53. The method of claim 49 wherein a toxicity-related population of proteins is identified.

54. The method of claim 49 wherein the agent is a chemical agent.

55. The method of claim 49 wherein a toxicity-related population of genes or proteins is identified by:
(a) measuring the level of expression of each member of a multiplicity of genes or proteins in the living thing, contacted with the agent, to yield a multiplicity of expression values;
(b) measuring the level of expression of each member of the same multiplicity of genes or proteins in a reference living thing, that is not contacted with the agent, to yield a multiplicity of reference expression values; and
(c) comparing the multiplicity of expression values with the multiplicity of reference expression values to identify a toxicity-related population of genes or proteins, wherein each individual gene or protein has an expression value in response to the agent that is significantly different from the corresponding reference expression value.

56. The method of claim 49 wherein the expression pattern of the toxicity-related population of genes or proteins appears in the living thing before the occurrence of the undesirable biological response in response to the agent.

57. The method of claim 49 wherein the undesirable biological response does not occur in the living thing.

58. The method of claim 49 wherein the living thing consists essentially of epididymal white adipose tissue.

59. The method of claim 49 wherein the undesirable biological response is selected from the group consisting of increased blood plasma volume, increased heart size, increased blood glucose concentration and increased total cholesterol.

60. The method of claim 49 further comprising:
(a) contacting a living thing with an agent that is known to elicit a desirable biological response and an undesirable biological response in the living thing, wherein elicitation of the desirable biological response is mediated by a first target molecule, and elicitation of the undesirable biological response is mediated by a second target molecule;
(b) identifying a population of genes or proteins that yields an expression pattern that correlates with the occurrence of the desirable and undesirable biological responses caused by the agent;
(c) contacting a modified living thing with the agent, wherein the modified living thing is a member of the same species as the living thing and does not include any functional second target molecules;
(d) identifying an efficacy-related population of genes or proteins that yields an expression pattern that correlates with the occurrence of the desirable biological response caused by the agent; and
(e) comparing the population of genes or proteins identified in step (b) with the efficacy-related population of genes or proteins identified in step (d) to identify a toxicity-related population of genes or proteins that yields an expression pattern that correlates with the occurrence of the undesirable biological response caused by the agent.

61. The method of claim 60 wherein the first target molecule is a PPARγ receptor and the second target molecule is a PPARα receptor.

62. A method for identifying a classifier population of genes or proteins, wherein the method comprises the steps of:
(a) contacting a living thing with a first reference agent that is known to cause a first biological response;
(b) identifying a first population of genes or proteins that yields an expression pattern that correlates with the occurrence of the first biological response caused by the first reference agent;
(c) contacting a living thing with a second reference agent that is known to cause a second biological response, wherein the living thing is the same living thing that is contacted with the first reference agent, or is a different living thing that is a member of the same species as the living thing that is contacted with the first reference agent;
(d) identifying a second population of genes or proteins that yields an expression pattern that correlates with the occurrence of the second biological response caused by the second reference agent; and
(e) comparing the first population of genes or proteins to the second population of genes or proteins and thereby identifying a classifier population of genes or proteins that produces an expression pattern that most clearly distinguishes between the first reference agent and the second reference agent.

63. The method of claim 62 wherein the living thing is a mammal.

64. The method of claim 62 wherein the living thing is a human being.

65. The method of claim 62 wherein a classifier population of genes is identified.

66. The method of claim 62 wherein a classifier population of proteins is identified.

67. The method of claim 62 wherein the agent is a chemical agent.
